(19) United States
(12) Patent Application Publication
(10) Pub. No.: US 2017/0009238 A1
(43) Pub. Date: Jan. 12, 2017

(54) System of interdependent anticancer antisense oligonucleotides targeting mRNAs the targets of which are NADPH-dependent, combined with one or more inhibitors of the Pentose Phosphate Pathway to deplete NADPH

(71) Applicants: Jonathan Wesley Nyce, Lansdale, PA (US); Alexander Trent Nyce, Lansdale, PA (US)

(72) Inventors: Jonathan Wesley Nyce, Lansdale, PA (US); Alexander Trent Nyce, Lansdale, PA (US)

(21) Appl. No.: 14/793,698

(22) Filed: Jul. 7, 2015

(51) Int. Cl. C12N 15/113

(52) U.S. Cl. CPC C12N 15/1135; C12N 2310/11; C12N 2310/333; C12N 2310/3341

(57) ABSTRACT

It is becoming increasingly clear that the genome and epigenome of another particular cancer cell represents the end product of a vast array of selection events, creating an extremely heterogeneous metabolic landscape. Since genomic and epigenomic variability exists between different cells in a tumor, the target complexity becomes even more extreme when the entire cancer cell population is considered. The only way to overcome this target complexity is by developing complex treatment modalities capable of simultaneously interdicting multiple pathways critical to the growth and survival of the cancer cell population. We have developed a method to couple suppression of NADPH levels (by inhibition of the Pentose Phosphate Pathway) with a system of antisense oligonucleotides targeting NADPH-dependent enzymes critical to the cancer cell. In the preferred embodiment of this invention, the PPP inhibitor is administered systemically, and the antisense oligonucleotides locally, directly to the tumor.

### STATEMENT REGARDING FEDERALLY FUNDED RESEARCH OR DEVELOPMENT

Although the applicant has received several federal research grants in the past (e.g., RO1 CA47217; R29 CA47217), the work underlying this invention is unrelated to the work conducted under such grants.

### CROSS REFERENCE TO RELATED PATENTS

None

### SEQUENCE LISTING

File Attached. All sequences containing ADENINE are understood to exist in both unmethylated adenine and methylated adenine (6meA) forms, and in both RNA and DNA forms, both single and double stranded. Similarly, because some dogs may exhibit hypersensitivity to unmethylated CG dinucleotides, all sequences in the listing containing CG dinucleotides are understood to exist in both methylated and unmethylated forms.

### PRIOR DISCLOSURES

There have been no prior disclosures of this invention

### BACKGROUND OF THE INVENTION

1. Field of the Invention

The field is directed to compositions and methods of treating canine cancer comprising a system of antisense oligonucleotides designed to destroy specific messenger RNA (mRNA) targets of NADP-dependent enzymes or pathways, combined with an inhibitor of NADPH production, in order to synergize the anticancer effect of such combination.

2. Description of Related Art

Approximately 1.7 million Americans are diagnosed with cancer each year, and approximately 600,000 will die each year from cancer (National Cancer Institute, Annual Cancer Statistics, United States, http://seer.cancer.gov/statfacts/). Worldwide, about 14 million new cases of cancer are diagnosed annually, and more than 8 million die from cancer (World Cancer Research Fund International, http://www.wcrf.org/int/cancer-facts-figures/worldwide-data). It has been authoritatively stated that more than 100 years of research into cancer treatment has resulted in an increased life span of as little as 3 months for many, if not most forms of cancer. (See Siddhartha Mukherjee , The Emperor of All Maladies, a Biography of Cancer; Simon & Schuster, 2010). In dogs, the situation is even worse due to their increased cancer risk as compared to humans. (See Torres de la Riva, G et al, PLoS One, 2013, 8(2):e55937). There thus remains a critical need to augment current treatment modalities, and to devise new, better treatment modalities for both human and veterinary cancer.

(0003) Many of the targets of classical chemotherapy drugs, for example methotrexate (CAS 59-05-2; MTX) target an enzyme requiring NADPH as cofactor. In the case of MTX, this enzyme is dihydrofolate reductase (CAS 9002-0303; DHFR). DHFR is the rate limiting step in the one carbon pool, or folate pathway (FIG. 1a, b). This pathway was deemed an appropriate target for chemotherapy design because it produces virtually all of the cancer cell's purines, pyrimidines, and S-adenosylmethionine (SAM), all of which play critical roles in intermediary metabolism and cell division. However, MTX is a quite toxic molecule when it is delivered systemically. In some therapeutic regimens, MTX is administered in lethal doses, and then the patient is rescued with folinic acid (leucovorin; CAS 1492-18-8). Leucovorin bypasses the DHFR inhibition by MTX, and replenishes the folate pathway and all of its products.

Another NADPH-dependent enzyme is ribonucleotide reductase (CAS 9047-64-7sy; RNR). RNR is a critical enzyme of deoxynucleotide synthesis, converting ribonucleotides to their corresponding deoxyribonucleotides. It directly requires NADPH reducing equivalents to effect this reaction (FIG. 2). Hydroxyurea (CAS 127-07-1) is a classical RNR inhibitor, first synthesized in 1869. HU shows efficacy in a variety of hematologic and other malignancies, but resistance can develop by down regulation of the M2 component of RNR. (See McClarty, G A et. al., Biochemical and Biophysical Research Communications 154(3):975-981, 1988). (0005) Gemcitabine (CAS 95058-81-4), another inhibitor of RNR, has found use in the treatment of a variety of human cancers, but the development of resistance is a recurring problem (See Kim, M P and Gallick, E, Clinical Cancer Res 14:2247, 2008). Clearly, new methods to inhibit RNR are needed that can avoid the development of resistance that appear to inhibit the effectiveness of the small molecule approach.

Thymidine kinase is found to be upregulated in many different kinds of cancer, especially lymphoma. (See Alegre, M M et al, Cancer and Clinical Oncology 2(1):159, 2013) Thymidine kinase is also an NADPH-dependent enzyme because its substrate, thymidine, requires folate pathway products for its synthesis, and the folate pathway is highly NADPH-dependent, as noted above. Although TK is recognized as a viable target for anticancer therapy (See Byun, Y, Doctoral Dissertation, The Ohio State University, 2006) and much work is ongoing attempting to develop small molecule TK inhibitors, none have yet been developed.

(0007) Like TK, deoxycytidine kinase (CAS 9002-06-6; dCK) is also NADPH dependent because its substrate is produced in part using products of the folate pathway. dCK phosphorylates deoxycytidine, deoxyguanosine and deoxyadenosine to their monophosphate forms, and so is an important enzyme in nucleotide biosynthesis and cell division. 2′-chloro-2′-deoxyadenosine (Cladribine, CAS 4291-63-8) is an inhibitor of dCK with activity in leukemia, but clearly, the small molecule approach has not produced much in the way of active inhibitors of this enzyme critical to the cancer cell. One reason for this may be that dCK is required to activate some commonly used anticancer drugs such as gemcitabine and cytarabine (cytosine arabinoside, CAS). (See Staub, M and Eriksson, S, Cancer Drug Discovery and Development, Peters, G J ed., Humana Press, Inc., Totowa, N.J.). Our antisense oligonucleotides should not be used in settings where use of agents that require activation by dCK. However, treatment of most solid tumors do not use such agents.

Thymidylate Synthetase (CAS 9031-61-2; TS) is another indirectly NADPH-dependent enzyme, because its substrate, dUMP, is also dependent upon folate pathway products for its synthesis. Several current and classical drugs target TS, including 5-Fluorouracil (CAS 51-21-8), and its prodrug, capecitabine. The utility of these TS inhibitors is reduced substantially by their toxicity profile, which can be life threatening in some individuals (Saif, M W, Cancer Genomics Proteomics. 2013 March-April; 10(2):89-92).

Adenosine receptors are dependent upon NADPH because the purine backbone required for the synthesis of adenosine is manufactured in the folate pathway. Since adenosine, through its receptors, primarily the adenosine A2A and A1 receptors, appears to play a fundamental role in cancer (Fishman, P, Handbook Exp Pharmacol 193:399-441, 2013; doi: 10.1007/978-3-540-89616-9_14), for example as a main tumor effector of escape from host immune control, novel methods of inhibiting adenosine receptor function in cancer are needed.

Tyrosine kinases such as c-kit are NADPH dependent because they are regulated by NADPH Oxidase (Paletta-Silva, R, Int J Mol Sci 14(2):3683-3704, 2013). Palladia is a c-kit inhibitor that has found some use in the treatment of canine mast cell cancer, but the upregulation of c-kit in a variety of tumor types clearly indicates the need for additional c-kit inhibitor strategies.

One characteristic feature of cancer is dramatically enhanced de novo fatty-acid synthesis. (See Swierczynski, J et al, World J Gastroenterology 20(9):2279-2303, 2014). Fatty acid synthesis is heavily NADPH dependent, and the FASN gene is frequently found to be significantly upregulated in cancer (See Flavin, R et al, Future Oncol 6(4):551-562, 2010). Nevertheless, no reliable treatment based upon the inhibition of the FASN gene product has been discovered.

Farnesyl transferase (FT) and Geranylgeranyl transferase (GGT) enzymes, critical for the isoprenylation of Ras, Rab and other oncoproteins, are potently dependent upon NADPH because their substrates are products of the mevalonate pathway, the rate limiting enzyme of which is HMG CoA reductase. HMG CoA reductase is extremely rare in intermediary metabolism in requiring 2 mols of NADPH for each mol of mevalonic acid product produced (Schultz, S and Nyce, J W, cancer Res 51:6563-6567, 1991). Although there are several statin drugs available that inhibit HMG CoA reductase, methods to specifically inhibit only portions of this pathway, e.g., farnesylation of Ras, Geranylation of Rab, are sorely needed.

The inactivation of tumor suppressor genes by aberrant DNA methylation is a well-known problem and rivals mutation as a mechanism of carcinogenesis. (See Robert, M F, Nat Genet 33(1):61-5, 2003). Most of this aberrant DNA methylation is thought to be induced by one of the primary DNA methylation gene products, DNMT1. While demethylating agents do exist, e.g., 5-azacytidine (CAS 320-67-2) and 5-aza-2′-deoxycytidine (CAS 2353-33-5), they must be incorporated into DNA to exert cytotoxicity, which can be a source of mutation in normal tissues. Is DNA methylation an NADPH-dependent process? Yes, it is. The methyl donor in all such methylation reactions is SAM, another product of the heavily NADPH-dependent folate pathway. Without SAM, DNMT1 cannot function. New ways to reverse the inactivation of tumor suppressor genes by aberrant methylation of them by DNMT1, methods that do not involve mutation-inducing events like incorporation into the DNA, are greatly needed.

BCL2, an anti-apoptotic gene upregulated in many different cancer types, especially hematopoietic malignancies, is another NADPH-dependent enzyme. Thus, it has been shown that down regulation of the folate pathway reduces BCL2 activity, releasing its block on apoptosis. (See Lin, H. L. et al, British J Nutrition 95:870-878, 2006). Although a number of BCL2 inhibitors have reached clinical trials, none have yet shown an efficacy to toxicity ratio acceptable to the FDA.

EZH2 is a Polycomb group gene the product of which is responsible for mono-methylation, di-methylation and tri-methylation at histone H3K27. EZH2 is overexpressed in many tumors, and is well known to suppress the expression of tumor suppressor genes (Liu, T. P. et al, Anti-Cancer Drugs 26(2):139-147, 2015). EZH2 is NADPH-dependent by virtue of its requirement for SAM as cofactor in its methylation reactions. Compositions and methods to inhibit EZH2 in tumors could be very important clinically.

S-Adenosylhomocysteine hydrolase (SAHH) converts S-Adenosyl-L-homocysteine to adenosine and homocysteine. Since the adenosine and homocysteine moieties involved in this reversible reaction are products of the folate pathway, SAHH is an NADPH-dependent enzyme. It is a major source of adenosine, which inactivates the host immune response. SAHH is not considered a good target for small molecule approaches to cancer chemotherapy because systemic administration would have severe pathological consequences. New methods to overcome or mitigate toxicity when SAHH is targeted are urgently needed.

Alkaline phosphatase (CAS 9001-78-9; ALPP) is another NADPH-dependent enzyme because its substrates, deoxyribonucleotides, have their structural backbones formed as a result of folate pathway activity. ALPP can metabolize adenosine nucleotides into adenosine. Methods to inhibit ALPP and thereby reduce or prevent defeat of immune surveillance by adenosine secretion into the tumor microenvironment would be important additions to the cancer chemotherapy armamentarium.

Phosphoribosylpyrophosphate Synthase (CAS 9031-46-3; PRPS), a critical pathway toward the de novo synthesis of purines and pyrimidines required for cell division, is heavily NADPH dependent because its substrate PRPP is produced by the Pentose Phosphate Pathway (PPP). Inhibition of either glucose-6-phosphate dehydrogenase (CAS 9001-40-5; G6PD) or 6-Phosphogluconate dehydrogenase (CAS 9001-82-5; PGD) (or both), two major enzymes of the PPP, leads to PRPP depletion and interruption of nucleotide biosynthesis. Two isoforms of the PRPS gene exist. Compositions and methods to inhibit PRPP would represent novel and potentially very therapeutic additions to cancer chemotherapy.

Amidophosphoribosyl transferase (EC 2.4.2.14; APRT) represents the committed step toward purine biosynthesis. Like PRPS, it is an NADPH-dependent enzyme because PRPP is produced by the PPP, and would be rendered inactive by any diminution in the biosynthesis of NADPH. No inhibitors of APRT have yet been introduced to the clinic.

IMP Dehydrogenase (CAS, 9028-93-7; IMPD) is an important enzyme in the biosynthesis of both adenine and guanine nucleotides. Its substrate, inosine monophosphate, is the product of de novo purine biosynthesis in which the purine ring structure is synthesized in the NADPH-dependent folate pathway. While IMPD is a recognized target for immunosuppressive drugs, it has not made headway as an anti-cancer target because of toxicity when delivered systemically. Methods to overcome this toxicity are greatly needed.

Polymerase alpha (CAS 9012-90-2; Pol a) is expressed in high amounts in cancer cells to maintain their rates of cell division. Pol α is of course NADPH-dependent because all the deoxyribonucleotides used as its substrate are derived from the NADPH-dependent folate pathway. Like many of the DNA synthesis enzymes discussed above, Pol α represents a good target because it is highly expressed in replicating cancer cells, and not expressed in normal, fully differentiated cells. Yet no inhibitors of this important target have yet found clinical utility.

Antisense oligonucleotides represent a promising alternative to antibody and small molecule approaches to the treatment of disease, as the present author (Nyce, J and Metzger, J M, Nature 385:721-725, 1997) and others (See Pirollo. K F et al, Pharmacol & Therapeutics 99(1):55-77, 2003) have demonstrated. However, their successful application to the field of cancer has been extremely limited. Methods to enhance their activity would have great therapeutic potential.

Antisense oligonucleotides comprise short (generally 15-20 base), single-stranded DNA or RNA, with (generally) chemically modified backbone to reduce nuclease degradation, that are complimentary to some region of a target mRNA (See Geary, RS et al Adv Drug Deliv Rev. 2015). There are several forms of backbone modification, including the exchange of one of the oxygen molecules for sulfur to create phosphorothioate oligonucleotides. Phosphorothioate and most (but not all) of the other chemistries permit facile hybridization to the target mRNA, with the result that the RNA:DNA hybrid becomes a substrate for RNAase H, which degrades the mRNA, allowing the antisense oligonucleotide to diffuse away with the potential of binding to another target mRNA and repeating the process. Locked nucleic acid (LNA) technology permit shorter 12-14 base long antisense oligonucleotides, and these are assumed in our listings. (See Straarup, E M et al, Nucleic Acids Res 38(20):7100-7111, 2012).

Backbone chemistries to prevent/decrease nuclease degradation envisioned in this patent include, for either DNA or RNA oligonucleotides, phosphorothiate, phosphorodithioate, 2′-O-methyl, 2′-Omethoxy-ethyl, 2′-F-arabino nucleic acid, Peptide nucleic acid, Morpholino phosphoroamidate, Bridged (locked) nucleic acid, cyclohexene nucleic acid, and hexitol nucleic acid. (See FIG. 3).

Antisense oligonucleotides present several advantages over the small molecule approach to cancer therapeutics. For example, it is possible to locally deliver antisense oligonucleotides either by direct injection, inhalation, and other methods. While small molecules can be and are injected, inhaled, etc., they readily diffuse out of the injection/inhalation focus creating systemic toxicity. Oligonucleotides, on the other hand, diffuse much less readily, and in many local delivery scenarios may never leave the tumor. Toxicity can thus be limited to the tumor and tumor associated non-neoplastic cells, e.g., blood vessels, etc.

Another advantage of antisense technology is that specific targets associated with neoplastic and not differentiated normal cells can be selected for attack. For example, normal differentiated cells express very minor amounts of such DNA synthesis genes as ribonucleotide reductase, thymidine kinase, nucleoside kinases, etc, while cancer cells generally express high levels of these enzymes as a requirement for their more rapid rates of replication. Thymidine kinase, for example, is expressed at high levels needed for cell division in cancer cells, while differentiated normal cells, in general, express the much reduced amounts needed only for DNA repair.

Local delivery of antisense oligonucleotides may be the key for their successful use, as envisioned in this invention (see below). Certainly, systemic delivery of antisense oligonucleotides has been shown to have many difficulties. As an example, consider an antisense oligonucleotide designated G3139 developed by Genta, Inc to target Bcl-2. This antisense oligonucleotide was called Genasense, and was designed to be complementary to the first 6 codons of Bcl-2 mRNA. While initial results in Phase I/II trials for lymphoma were promising, a disappointing melanoma trial caused Genta to close its doors in 2012.

One of the present inventors has several patents in relevant fields.

U.S. Pat. No. 6,040,296 A (Nyce, J W, 2000) teaches the use of antisense oligonucleotides targeting adenosine receptors for treatment of disorders associated with bronchoconstriction and lung inflammation.

U.S. Pat. No. 5,994,315 A (Nyce, J W 1999) teaches the use of antisense oligonucleotides with low adenosine content to treat lung diseases.

U.S. Pat. No. 6,025,339 A (Nyce, J W, 2000) teaches the use of antisense oligonucleotides for the treatment of bronchoconstriction and lung inflammation.

U.S. Pat. No. 6,670,349 B1 (Nyce, J W, 2003) teaches the use of Dehydroepiandrosterone to lower adenosine levels to treat asthma.

U.S. Pat. No. 7,456,161 B2 (Nyce, J W, 2008), teaches the use of dehydroepiandrosterone to treat chronic obstructive pulmonary disease by reducing levels of adenosine in bronchial mucosa.

U.S. Pat. No. 8,951,527 B2 (Isenberg, J S et al, 2015) and EU Patent application WO 2012170250 (Moulton, J 2011) both inform the use as radiation protectants of morpholino antisense oligonucleotides targeting the human CD47 sequence, in which the inhibition of CD47 allows normal tissue within a radiation beam to survive otherwise damaging or lethal doses of radiation.

U.S. Pat. No. 8,710,020 B2 (Gleave, M E and Cormack, S D, 2014) teaches a method for using antisense oligonucleotides to reduce the expression of clusterin and thereby treat cancers in which clusterin play a major role.

Application WO2014153209 Al (Erin, L O et al (2014) teaches the use of antisense oligonucleotides targeting non-coding chimeric mitochondrial DNA in the treatment or prevention of relapsing or metastatic cancer.

U.S. Pat. No. 7,402,574 B2 (Iverson, P L et al, 2008) teaches the use of antisense oligonucleotides targeting the SNAIL transcript.

U.S. Pat. No. 7,569,551. B2 (Gleave, M et al, 2009) teaches the use of antisense oligonucleotides targeting Testosterone-repressed prostate message 2 (TRPM-2)

U.S. Pat. No. 5,641,754 A (Iversen, P L, 1997) teaches the cornbination of an antisense oligonucleotide and a hydroxyl-radical upregulator to treat cancer.

U.S. Pat. No. 6,727,230 B1 (Hutcherson, S. L., Glover, J M, 2004) teaches the use of phosphorothioate oligonucleotide analogs to stimulate an immune response.

EU Patent EP 1009822 A1, (Higenbottom, T, 2000) teaches the use of antisense oligonucleotides targeting ET-1 for the treatment of pulmonary hypertension.

U.S. Pat. No. 7,585,968 B2 (James G. Karras, Kenneth W. Dobie , 2009) teaches the use of antisense oligonucleotides targeting TARC for the treatment of airway hyper responsiveness and pulmonary inflammation.

U.S. Pat. No. 7,001,890 B1 (Wagner, H et al, 2006) teaches the combination of an oligonucleotide and a protein/peptide antigen for the purpose of vaccination.

Application WO2001025422 A2 (Bartelmez, S H and Iversen, PL (2001) teaches a method to treat cancer which comprises using an antisense oligonucleotide to increase the number of lineage committed progenitor cells.

Application WO1997011170 A1 (Zamecnik, P A, 1997) teaches the use of prostate-specific antisense oligonucleotides to treat prostate cancer.

Application WO2008058225 A2 (Brown, B D, 2008) teaches the use of an antisense oligonucleotide targeting BCL-2 for the treatment of cancer. This antisense did not work in clinical trials and represents a good example of why antisense oligonucleotides have experienced difficulty in their clinical development. Administered systemically, and without any method to secondarily interfere with the target protein (and not just the mRNA), these molecules cannot overcome the heterogeneity and plasticity of tumor cell populations.

U.S. Pat. No. 5,801,154 A (Baracchini, E et al, 1998) teaches the use of antisense oligonucleotides targeting the Multi-drug resistance-associated protein to treat or prevent the occurrence of multi-drug resistance during chemotherapy.

U.S. Pat. No. 6,017,898 A (Pietrzkowski, Z et al, 2000) teaches the use of antisense oligonucleotides targeting IL-8 and the IL-8 receptor for the treatment of cancer.

Chinese patent CN1120179 C ( [symbol] , 2003) teaches the use of an antisense oligonucleotide targeting c-raf to treat cancer.

Application CA2471967 A1, (Lopez-Bernstein et al, 2003) teaches the use of antisense oligonucleotides targeting Wt1 for the treatment of cancer.

U.S. Pat. No. 5,691,317 A, (Yoon, S C C, 1997) teaches the use of an antisense oligonucleotide targeting the RI-α subunit of cyclic AMP-dependent protein kinase. This is an early entry into the field of antisense oligonucleotides targeting cancer genes.

U.S. Pat. No. 7,704,968 B2 (Nerenberg, M I, 2010) teaches the suppression of nuclear factor-kB for treating inflammatory conditions.

A major flaw in all of these patents and applications, a flaw which places severe restraints upon their clinical utility, is that they depend upon an antisense oligonucleotide alone to reduce the impact of the target upon the disease process. What is needed to move antisense technology forward is a method or methods to reduce the production of the target protein using antisense technology, preferably by local administration directly to the tumor, while simultaneously inhibiting the protein target, preferably by a non-toxic, systemic route of administration. While this could be effected using local administration of antisense oligonucleotides and systemic administration of classical chemotherapy agents, such a method would still suffer from the systemic toxicity inherent in the chemotherapy drugs. A better approach would be to find a class of cancer targets that all have one common dependency, for example, targets that all require the same cofactor, reductant, vitamin, etc., and exploit that dependency by causing its depletion, so that upon application of antisense oligonucleotides against the targets, such targets are simultaneously suppressed at both the protein and the mRNA levels.

### BRIEF SUMMARY OF THE INVENTION

This invention comprises a system of antisense oligonucleotides targeting canine genes whose enzyme products are NADPH-dependent, combined with one or more inhibitors of the pentose phosphate pathway (PPP), the major source of cellular NADPH. The object is to synergize the inhibition of target gene products by (a) reducing the amount of target protein via antisense oligonucleotide interference with its translation/production, and (b) inhibiting remaining target protein via NADPH-depletion. An additional goal is to maximize tumor toxicity and minimize systemic toxicity by delivering the antisense oligonucleotides locally, as, for example, with direct injection into the tumor, while the PPP inhibitor can be delivered systemically or locally.

The NADPH-dependent genes targeted by this system include thymidine kinase (TK), thymidylate synthetase (TS), dihydrofolate reductase (DHFR), ribonucleotide reductase (RNR), c-kit tyrosine kinase, fatty acid synthase (FASN), the anti-apoptotic gene BCL2, the polycomb gene EZH2, the DNA methyltransferase gene DNMT1, farnesyl transferase (FT), geranylgeranyl transferase (GGT), Phosphoribosylpyrophosphate Synthase (PRPS), Amido-phosphoribosyl transferase (APRT), IMP Dehydrogenase (IMPD), and Polymerase a (Pol α).

The PPP inhibitors include Dehydroepiandrosterone (DHEA) and any of its congeners, analogs, precursors, products, and salts; and/or antisense oligonucleotides targeting Glucose-6-phosphate dehydrogenase (G6PD) and/or 6-Phosphogluconate Dehydrogenase (PGD).

Where DHEA is used as the PPP inhibitor, it may be necessary to reconstitute certain metabolites depleted as a result of PPP inhibition, as described in a parallel United States Patent Application filed this same date. Such depleted metabolites that may need to be reconstituted include tetrahydrobiotperin, N-6-isopentenyladenosine, a nitric oxide donor such as potassium nitrate, folinic acid (or products of the folate pathway), ubiquinone +tocotrienols, and certain monoamine precursors and cofactors including L-DOPA, 5-Hydroxytryptophan, pyridoxine, SAMe, ascorbate, pantothenic acid, and zinc.

Where DHEA or one of its congeners is the PPP inhibitor, it may be administered by any of several techniques, but injected, inhaled, transdermal and oral methods are preferred. The antisense oligonucleotides may be administered by any of several techniques, but injected, inhaled and transdermal methods are preferred.

It may sometimes be necessary to select sequences that are not 100% complimentary to the human version of the respective genes so as to avoid the possibility of human exposure during development and/or clinical usage, and such selection for canine-specific antisense oligonucleotides that will not interact with human mRNAs (and thereby represent a threat to humans exposed to it) represents one aspect of this invention.

In addition to DNA based antisense oligonucleotides, this invention also envisions the use of small interfering RNA (RNAi), Dicer-substrate siRNA, and microRNA oligonucleotides based upon the sequences in our listing. Accordingly, even though the sequences listed are single-stranded, if they are used in some of the RNA procedures, it is understood that the listed sequences can represent one half of a double stranded nucleic acid the bases of which are participating in hydrogen bonding between the counter opposing strands.

### BRIEF DESCRIPTION OF FIGURES AND TABLES

FIG. 1(a) is a representation of the DHFR pathway illustrating the dependency of DHFR upon NADPH, 1 (b) illustrates how folate intermediates are required for purine and pyrimidine biosynthesis, for DNA, RNA protein and lipid methylation reactions, etc.

FIG. 2 is a representation of the RNR pathway illustrating the dependency of RNR upon NADPH.

FIG. 3 is a detailed illustration of the various oligonucleotide chemistries available. The present invention envisions using any or all of these chemistries.

FIG. 4. (a) Effects of adenosine A1 receptor antisense oligodeoxynucleotide upon PC50 values in asthmatic Rabbits. PC50 adenosine values were determined before and after intratracheal administration of aerosolized antisense oligonucleotide targeting the adenosine A2 receptor (A2AS), or a control oligonucleotide in which the A1 receptor sequence was randomized (A1MM, A1MM2)). After a two-week rest period between parts of the experiment, rabbits were then crossed over, with those that had received the A1 antisense oligonucleotide now receiving the randomized oligonucleotide, and those that had received the randomized oligonucleotide now receiving the A1 antisense oligonucleotide. A1MM2 animals constituted a separate group. (b), Data summary. Results are presented as the mean±s.e.m. Significance was determined by repeated measures ANOVA and Tukey's protected t test. Asterisks indicate a significant difference from all other groups, P<0.01. PC50 adenosine, the amount of adenosine required to reduce lung compliance by 50%.

FIG. 5. Specificity of action of Adenosine Al receptor antisense oligodeoxynucleotide A1AS. Airway smooth muscle tissue was dissected from rabbits administered a total of 20 mg A1AS or A1MM in four divided doses over 48 hr. Plasma membrane fractions were prepared. (a), Saturation isotherm of [³H]DPCPX (a potent selective antagonist of the adenosine Al receptor) binding to allergic rabbit lung plasma membrane from A1AS (filled circles) and A1MM (open circles) allergic rabbits showing an approximate 75% decrease in adenosine A1 receptor number in airway smooth muscle from A1AS-treated animals. (b), Scatchard plot saturation isotherm from (a) indicating a single class of binding sites; A1AS (filled circles), A1MM (Open circles). Please note that the error bars are an artifact of the printing program; Scatchard plots have no error bars. (c), Saturation isotherm of [³H]NPC17731 (a selective bradykinin B2 receptor antagonist) binding to allergic rabbit lung plasma membrane from AlAS (open squares) and A1MM-treated (filled squares) allergic rabbit showing no change in Bradykinin B2 receptor number in airway smooth muscle of A1AS-treated animals. (d), Scatchard plot of saturation isotherm from c indicating a single class of binding sites. A1AS (open squares), A1MM (filled squares). Error bars are an artifact of the drawing program.

FIG. 6. Specificity of action of bradykinin B2 receptor antisense oligonucleotide B2AS. Airway smooth muscle tissue was dissected from rabbits administered 20 mg B2AS or B2MM in four divided doses over 48 hr. Plasma membrane fractions were prepared. (a), Saturation isotherm of [³H]NPC17731 binding to allergic rabbit lung plasma membrane from B2ASO (open triangles) and B2MM-treated (filled triangles) allergic rabbits showing an approximate 40% decrease in bradykinin B2 receptor number in airway smooth muscle from B2AS-treated animals. (b), Scatchard plot of saturation isotherm from a indicating a single class of binding sites; B2AS (open triangles), B2MM (filled triangles). (c), Saturation isotherm of [³H]DPCPX binding to allergic rabbit lung plasma membrane from B2AS (open diamonds) and B2MM (filled diamonds) allergic rabbits showing no change in adenosine A1 receptor number. (d), Scatchard plot of saturation isotherm from c indicating a single class of binding sites; B2AS (open diamonds), B2MM (filled diamonds).

FIG. 7. The effect of antisense and mismatched/randomized oligodeoxynucleotides on allergen-induced airway obstruction and bronchial hyper responsiveness in allergic rabbits. The allergen employed was house dust mite allergen (Dermatophagoides farina). (a), Effect of AlAS antisense oligodeoxynucleotide upon allergen-induced airway obstruction. Allergen only (filled circles); allergen+antisense (open circles). As calculated from the area under the curve, A1as significantly inhibited allergen-induced airway obstruction (55%, P<0.05; repeated measures ANOVA and Tukey's protected t test. (b), Lack of effect of mismatch control A1MM on allergen-induced airway obstruction. Allergen only (filled circles); allergen=mismatch control (open circles). (c), Effect of A1AS antisense oligodeoxynucleotide on allergen-induced bronchial hyperresponsiveness. As calculated from the PC50 histamine, A1AS significantly inhibited allergen-induced bronchial hyperresponsiveness in allergic rabbits (61%, P<0.05; repeated measures ANOVA and Tukey's protected t test). (d), Lack of effect of A1MM mismatch control on allergen-induced bronchial hyperresponsiveness. Dynamic compliance (Cdyn) is the change in the volume of the lungs divided by the change in the alveolar-distending pressure during the course of a breath.

Table 1. Binding characteristics of the adenosine A1 receptor-selective ligand [³H]DPCPX and the bradykinin B2-selective ligand [³H]NPC17731 in membranes isolated from airway smooth muscle of A1 adenosine receptor and B2 Bradykinin receptor antisense- and mismatch control-treated allergic rabbits. Treatment values refer to total oligonucleotide administered in four equivalently divided doses over a 48 hour period. Significance was determined by repeated measures ANOVA and Tukey's protected t test; N=4-7 for all groups. All assays were performed in triplicate. *Significantly different from mismatch control and saline-treated groups, P<0.001. ** Significantly different from mismatch control and saline treated groups, P<0.05.

### DETAILED DESCRIPTION OF THE INVENTION

A preferred embodiment of the invention is a pharmaceutical composition comprising one or more antisense oligonucleotides drawn from the sequence listing, combined with an inhibitor of the PPP. The NADPH-dependent targets identified in the sequence listing include thymidine kinase (TK), thymidylate synthetase (TS), dihydrofolate reductase (DHFR), ribonucleotide reductase (RNR), the adenosine receptors A1 and A2A, c-kit tyrosine kinase, fatty acid synthase (FASN), the anti-apoptotic gene BCL2, the polycomb gene EZH2, the DNA methyltransferase gene DNMT1, farnesyl transferase (FT), geranylgeranyl transferase (GGT), S-Adenosylhomo-cysteine hydrolase (SAHH), alkaline phosphatase (ALP), Phosphoribosylpyro-phosphate Synthase (PRPS), Amidophosphoribosyl transferase (APRT), IMP Dehydrogenase (IMPD), and Polymerase a (pol α).

If sequencing of the tumor is performed before treatment, drivers may be identified and groups selected for antisense targeting accordingly. If sequencing is not performed prior to treatment, a series of treatments in which one or more antisense oligonucleotides is administered alone or in combination, may be used to address NADPH-dependent drivers.

The sequence list is comprised of NADPH-dependent drivers found in whole or in part in most neoplasms, including mast cell tumors, mammary gland tumors, soft tissue sarcomas, anal gland tumors, osteosarcomas (primary and metastatic), lung tumors (primary and metastatic), melanomas (primary and metastatic), fibrosarcomas, hemangiosarcomas (primary and metastatic), nasal cavity cancers, squamous cell carcinomas, tumors of the CNS, gastric tumors, intestinal cancers, hepatic cancers, kidney cancers, prostate cancers, urinary tract cancers, testicular cancers, pancreatic cancers, anal gland tumors, and lymphosarcoma.

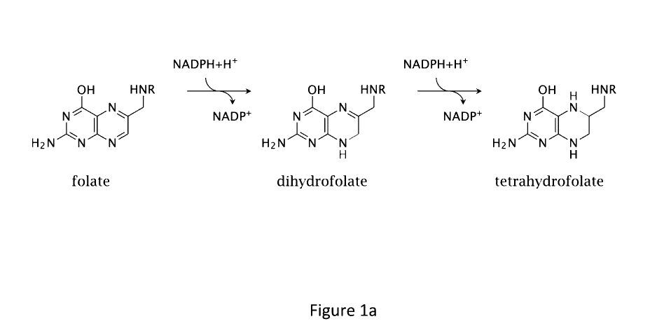

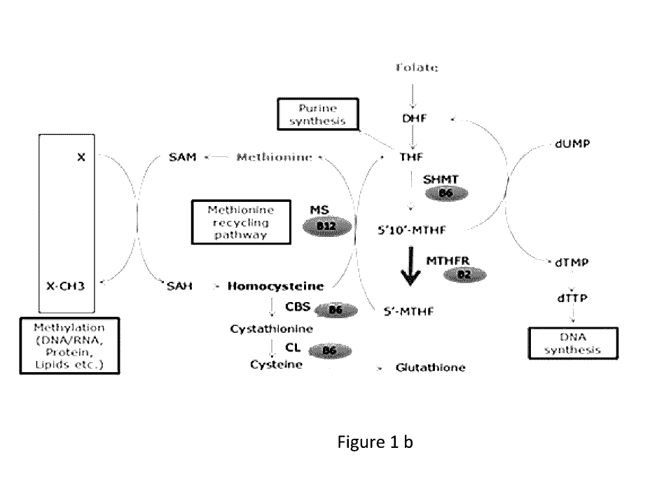

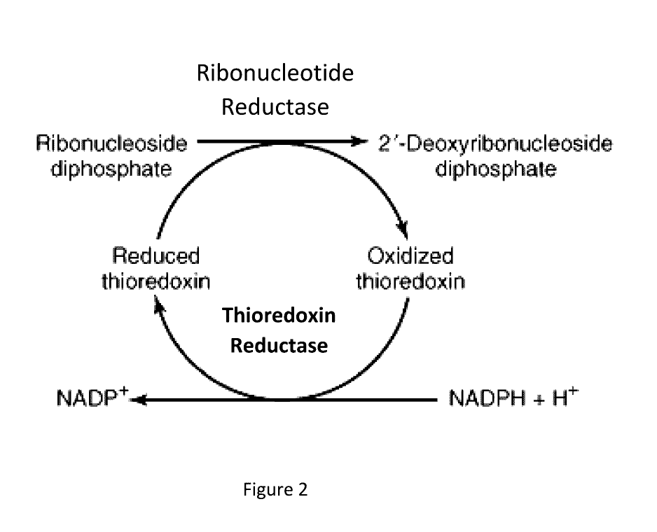

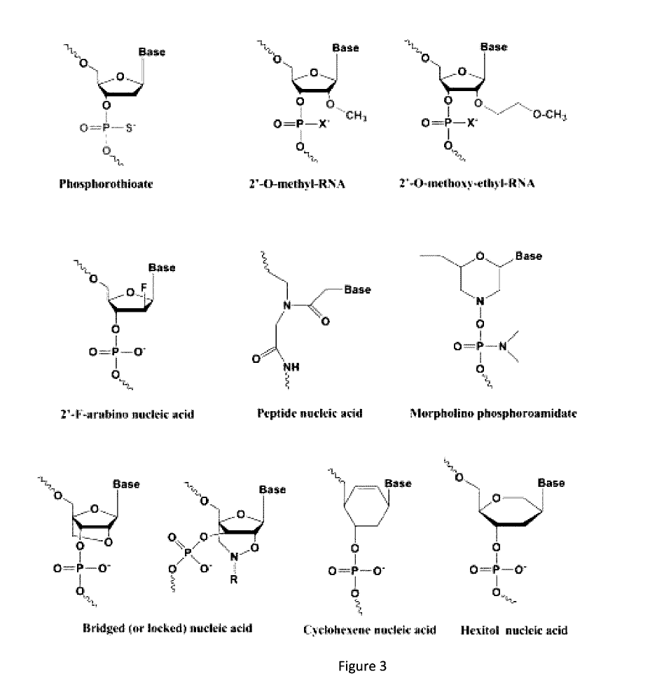

The backbone chemistries to prevent/slow nuclease degradation envisioned in this patent include for either DNA or RNA or DNA/RNA mixed oligonucleotides, phosphorothiate, 2′-O-methyl, 2′-O-methoxy-ethyl, 2′-F-arabino nucleic acid, Peptide nucleic acid, Morpholino phosphoroamidate, Bridged (locked) nucleic acid, cyclohexene nucleic acid, and hexitol nucleic acid. (See FIG. 1). These chemistries can be applied to all the nucleotides in the sequence, or to a more limited number, even as few as 1. These chemistries can also be mixed, such that portions of the antisense oligonucleotide can utilize one chemistry, and other portions another chemistry, in any combination or sequence desired.

In addition to DNA based antisense oligonucleotides, this invention also envisions the use of small interfering RNA (RNAi), Dicer-substrate siRNA , and microRNA oligonucleotides based upon the sequences in our listing. For the purposes of this invention, then, the words antisense oligonucleotide are meant to imply the use of any or all of these technologies.

A preferred embodiment of an antisense oligonucleotide targeting canine TK-1 can be drawn from sequence ID number 283, having the sequence 5′-gcaggttgat gcagctcatg g-3′, but other of the TK-1 antisense sequences in the list may be used as well.

A preferred embodiment of an antisense oligonucleotide targeting canine TS can be drawn from sequence ID number 1119, having the sequence 5′-gggcgggcat ggcgcgggcg g-3′, but other of the TS antisense sequences in the list may be used as well.

A preferred embodiment of an antisense oligonucleotide targeting canine DHFR can be drawn from sequence ID number 327, having the sequence 5′-ggctgcggcc ccatttcatg t-3′, but other of the DHFR antisense sequences in the list may be used as well.

A preferred embodiment of an antisense oligonucleotide targeting canine RNRM1 can be drawn from sequence ID number 741, having the sequence 5′-cgcttgatca cgtgcatcgc g-3′, but other of the RNR antisense sequences in the list may be used as well.

A preferred embodiment of an antisense oligonucleotide targeting canine RNRM2 can be drawn from sequence ID number 792, having the sequence 5′-cgcggacgga gagcatggcg g-3′, but other of the RNR antisense sequences in the list may be used as well.

A preferred embodiment of an antisense oligonucleotide targeting canine RNRM3 can be drawn from sequence ID number 893, having the sequence 5′-cctgtttata cattttccaa a-3′, but other of the RNR antisense sequences in the list may be used as well.

A preferred embodiment of an antisense oligonucleotide targeting canine adenosine A2a receptor can be drawn from sequence ID number 107, having the sequence 5′-gcccatggtg gacatggctg c-3′, but other of the adenosine A2a receptor antisense sequences in the list may be used as well.

A preferred embodiment of an antisense oligonucleotide targeting canine adenosine A1 receptor can be drawn from sequence ID number 468, having the sequence 5′-ggcgggcggc atggcaggcg c-3′, but other of the adenosine A1 receptor antisense sequences in the list may be used as well.

A preferred embodiment of an antisense oligonucleotide targeting canine c-kit tyrosine kinase can be drawn from sequence ID number 249, having the sequence 5′-gcgagcgcct ctcatcgcgg t-3′, but other of the c-kit tyrosine kinase antisense sequences in the list may be used as well.

A preferred embodiment of an antisense oligonucleotide targeting canine FASN can be drawn from sequence ID number 1015, having the sequence 5′-cctcctccat ggctgctctg c-3′, but other of the FASN antisense sequences in the list may be used as well.

A preferred embodiment of an antisense oligonucleotide targeting canine BCL2 can be drawn from sequence ID number 571, having the sequence 5′-gcccagcgtg cgccatcctc c-3′, but other of the BCL2 antisense sequences in the list may be used as well.

A preferred embodiment of an antisense oligonucleotide targeting canine EZH2 can be drawn from sequence ID number 672, having the sequence 5′-ggcccatgat tattctgcgc c-3′, but other of the EZH2 antisense sequences in the list may be used as well.

A preferred embodiment of an antisense oligonucleotide targeting canine DNMT1 can be drawn from sequence ID number 671, having the sequence 5′-ggcccatgat tattctgcgc cc-3′, but other of the DNMT1 antisense sequences in the list may be used as well.

A preferred embodiment of an antisense oligonucleotide targeting canine FT can be drawn from sequence ID number 684, having the sequence 5′-cacgaactcc atgctggcgg c-3′, but other of the FT antisense sequences in the list may be used as well.

A preferred embodiment of an antisense oligonucleotide targeting canine ALPP (alkaline phosphatase) can be drawn from sequence ID number 993, having the sequence 5′-gggtcaggag cattgcaggg c-3′, but other of the ALPP antisense sequences in the list may be used as well.

A preferred embodiment of an antisense oligonucleotide targeting canine GGT 1α can be drawn from sequence ID number 1077, having the sequence 5′-gcgcccgtgc atggtgccgg c-3′, but other of the GGT antisense sequences in the list may be used as well.

A preferred embodiment of an antisense oligonucleotide targeting canine GGT 1β can be drawn from sequence ID number 1063, having the sequence 5′-cgccgctcct ctacatcgaa c-3′, but other of the GGT antisense sequences in the list may be used as well.

A preferred embodiment of an antisense oligonucleotide targeting canine SAHH can be drawn from sequence ID number 940, having the sequence 5′-cagtttgtcc gacatgctgg c-3′, but other of the SAHH antisense sequences in the list may be used as well.

A preferred embodiment of an antisense oligonucleotide targeting canine PRPS1 can be drawn from sequence ID number 1046, having the sequence 5′-cggcatcttg ggtgcctacc c-3′, but other of the PRPS antisense sequences in the list may be used as well.

A preferred embodiment of an antisense oligonucleotide targeting canine PRPS2 can be drawn from sequence ID number 1039, having the sequence 5′-ccgatgacat ccttctccga t-3′, but other of the PRPS2 antisense sequences in the list may be used as well.

A preferred embodiment of an antisense oligonucleotide targeting canine APRT can be drawn from sequence ID number 1130, having the sequence 5′-cctccagctc catgtcgctg cc-3′, but other of the APRT antisense sequences in the list may be used as well.

A preferred embodiment of an antisense oligonucleotide targeting canine IMPD can be drawn from sequence ID number 1135, having the sequence 5′-gcggcccctc catgcggagg c-3′, but other of the IMPD antisense sequences in the list may be used as well.

A preferred embodiment of an antisense oligonucleotide targeting canine IMPD2 can be drawn from sequence ID number 1160, having the sequence 5′-cgcatgcgca aagcgcgccg t-3′, but other of the IMPD antisense sequences in the list may be used as well.

A preferred embodiment of an antisense oligonucleotide targeting canine Pol a can be drawn from sequence ID number 1023, having the sequence 5′-cgccgtctac cagcgccatg g-3′, but other of the Pol α antisense sequences in the list may be used as well.

In a preferred embodiment of this invention, the antisense oligonucleotides can be administered in doses ranging from, preferentially, 0.1 mg/kg to 50 mg/kg; more preferentially 1 mg to 30 mg/kg; and most preferentially, 2 mg to 20 mg/kg. In a preferred embodiment of the invention the antisense oligonucleotides are administered locally, for example by direct injection into a tumor; by inhalation when tumors exist in the lung; by means of a transdermal cream if injection is not possible, for example in tumors at risk of catastrophic bleeding such as hemangioendotheliomas; and by intrathecal injection for tumors in the brain.

A variety of microorganisms do not methylate the majority of CpG dinucleotides in their DNA. Bacterial genomes also contain 6-methyladenine replacing adenine in some sites in their DNA. These non-self “signals” activate Toll receptors and initiate an immune response directed to the vicinity where they are detected. A preferred embodiment of this invention therefore involves the substitution of any number of adenosine moieties with 6-methyladenosine moieties to stimulate a local immune response. Because some dogs may show heightened response to unmethylated CpGs, a preferred embodiment of this invention, for use in dogs with hypersensitivity to unmethylated CpGs, is the substitution of 5 mC for C in such unmethylated CpGs,

A preferred embodiment of the PPP inhibitor is DHEA, and/or any of its congeners capable of inhibiting the PPP, such as DHEA sulfate, DHEA sulfatide, any DHEA salt, 7-keto-DHEA, 3 acetyl-7-oxo DHEA, DHEA acetate, the DHEA metabolites androstenediol and androstendione, and the less metabolizable analogs fluasterone (CAS 112859-71-9) and the brominated analog of fluasterone.

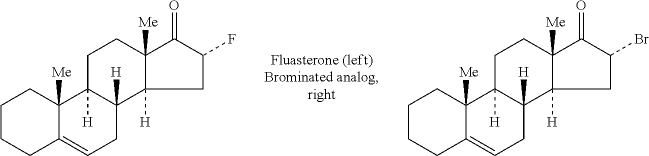

An additional preferred embodiment of the PPP inhibitor is an antisense oligonucleotide targeting canine G6PD, which antisense oligonucleotide can be drawn from sequence ID number 720, having the sequence 5′-cccgccggct catttaacca g-3′, but other of the G6PD antisense sequences in the list may be used as well.

An additional preferred embodiment of the PPP inhibitor is an antisense oligonucleotide targeting canine PGD, which antisense oligonucleotide can be drawn from sequence ID number 504, having the sequence 5′-cggcctcggc catggcggcg g-3′, but other of the PGD antisense sequences in the list may be used as well.

An additional preferred embodiment of the PPP inhibitor is a combination of a canine G6PD antisense oligonucleotide with a canine PGD antisense oligonucleotide, such that both distal steps in the PPP are inhibited. Also, DHEA or one of its PPP-inhibiting congers can be administered systemically, and either G6PD antisense oligonucleotide or PGD antisense oligonucleotide, or both, can be administered locally, so as to maximize inhibition of the PPP.

Where systemic DHEA or a congener is used to inhibit the PPP, an additional preferred embodiment of the invention is supplementation of DHEA-treated dogs with metabolites that may, as a consequence of DHEA-mediated NADPH-depletion, become depleted physiologically and which may need to be replenished to maintain optimum health in the treated dog. Such supplementation may include tetrahydrobiotperin (BH4); isopentenyladenosine (IPA); folinic acid or certain products of the folate pathway (e.g., SAM, adenine, guanine, and uracil and/or any of their nucleosides or nucleotides); a nitric oxide donor such as Potassium Nitrate; ubiquinone and/or tocotrienols to maintain healthy ubiquinone levels; and certain monoamine precursors and cofactors such as hydroxytryptophan, L-DOPA, melatonin, pyridoxine, ascorbate, and pantothenic acid.

A preferred embodiment of the invention is target selection from the sequence list based upon Next Generation RNA sequencing such that those targets on the list that are expressed in aberrant, greater than normal levels are identified. Such sequencing uses reverse transcription of RNA isolated from canine tumors (and control tissue, where available), a panel of PCR primers specific for the listed targets to identify and quantitate their contribution to the transcriptome, and annealing of barcode DNA to individual tumor samples so that the transcriptomes from several different animals can be multiplexed, i.e., mixed together during the sequencing run. Output is then extracted for each animal by barcode decoding, and the contribution of different targets to the transcriptome is analyzed. Where such RNA sequencing is not possible, various antisense oligonucleotides selected from the list may be used sequentially or in groups to treat the cancer in a blind fashion.

A preferred embodiment of the invention is the local administration of the antisense oligonucleotide or nucleotides, in order to reduce systemic toxicity, and the systemic administration of DHEA or its congeners, although local routes of DHEA administration may be preferred in some situations. If one of the G6PD antisense oligonucleotides, and/or one of the PGD antisense oligonucleotides are selected for use, these will be administered locally, by inhalation, injection (intratumor, intrathecal, suppository, subcutaneous), or transdermal routes.

Preparation of antisense oligonucleotides is accomplished by any of several published techniques. A variety of chemical modifications can be used to inhibit nuclease degradation in vivo. For phosphorothioate oligonucleotides we use standard phosphoramidite chemistry, substituting iodine reagent with either Beaucage reagent or Sulfurizing Reagent 2 (Glen Research, Sterling, Va.), using an ABI Expedite 8909 High Throughput DNA Synthesizer equipped with a 16 column MOSS (Multiple Oligonucleotide Synthesis System) unit. Purification is on 20 gram GlenPak cartridges as described (Glen Research).

Phosphonoacetate monomers may be included in phosphorothioate oligonucleotides, or used as a stand alone chemistry. In fully modified oligos, the non-aqueous oxidizer camphorsulfonyloxaziridine is used, and oxidation precedes capping in the synthesis cycle. The use of such monomers greatly enhances the uptake of oligonucleotides into cells, and accelerates RNAase H degradation. The standard protocol for cleavage and deprotection requires a two-step method with pretreatment using 1,8-diazabicyclo[5.4.0]undec-7-ene (to deprotect the dimethylcyanoethyl protecting groups and prevent alkylation of the bases during deprotection) and subsequent cleavage using methylamine.

Methyl Phosphonamidite chemistry may be used in DNA synthesizers following conventional CE Phosphoramidite protocols to produce oligonucleotides containing one or more methyl phosphonate linkages.

Replacing two non-bridging oxygen atoms with sulfur atoms in a DNA phosphodiester linkage creates a phosphorodithioate (PS2) linkage. Like natural DNA, the phosphorodithioate linkage is achiral at phosphorus. This analog is completely resistant to nuclease degradation but forms complexes with DNA and RNA with somewhat reduced stabilities.

After purification, the antisense oligonucleotides useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. They can be administered orally, as a liquid, a lozenge, a capsule or a tablet. Flavor additives, stabilizers, solubilizing agents and flow enhancers and the like can be used to modify its flavor, activity, solubility and compressibility. They may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intratumor, intracranial or intrathecal.

The antisense oligonucleotides useful for this invention can be formulated either alone or in combination with any or all of the other antisense oligonucleotides of this invention.

As used in the context of this invention, co-administration refers to temporal proximity. Thus, the agents described as “co-administered” may be administered exactly together; they may be delivered one or more before the other(s); they may be delivered one or more after the other(s); or some combination of the above.

Antisense oligonucleotides can be supplied for clinical use in dried form, to be taken up in a suitable buffer depending upon the route of administration, immediately before use. Alternatively, they can be shipped in sterile buffered aqueous solution, but a pH above 7.0 (e.g., 7.5-8.0) is needed to maintain stability. Depending upon the sequence structures, if several antisense oligonucleotides are administered together in the same solution, structures with extensive regions of base pairing (>30-40%) may require heating to destabilize hydrogen bonding. One procedure is to rapidly heat the mixture of oligonucleotides to 100° C., then plunge them rapidly into ice water. This procedure should be sufficient to destabilize most hydrogen bond-induced aggregation in mixtures of antisense oligonucleotides with moderate base pairing potential.

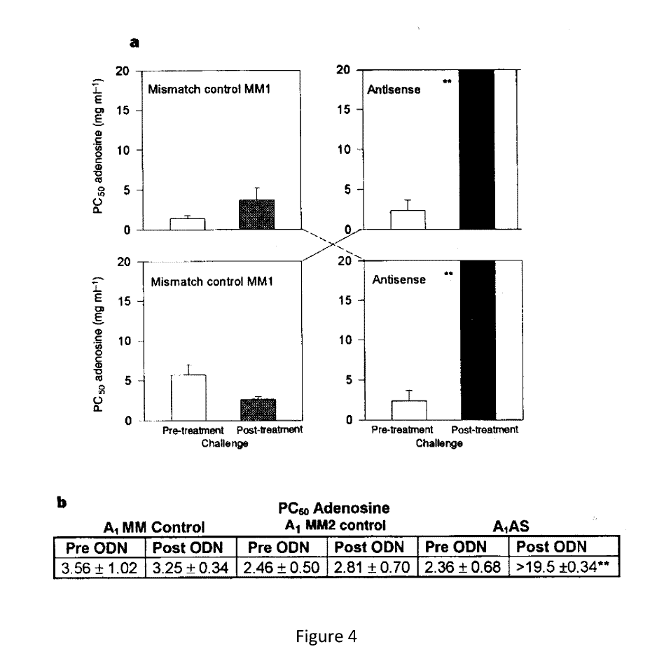

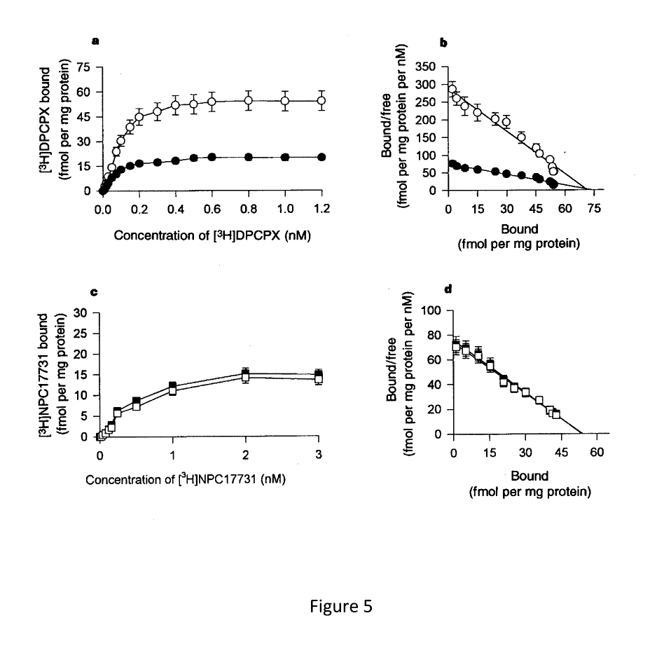

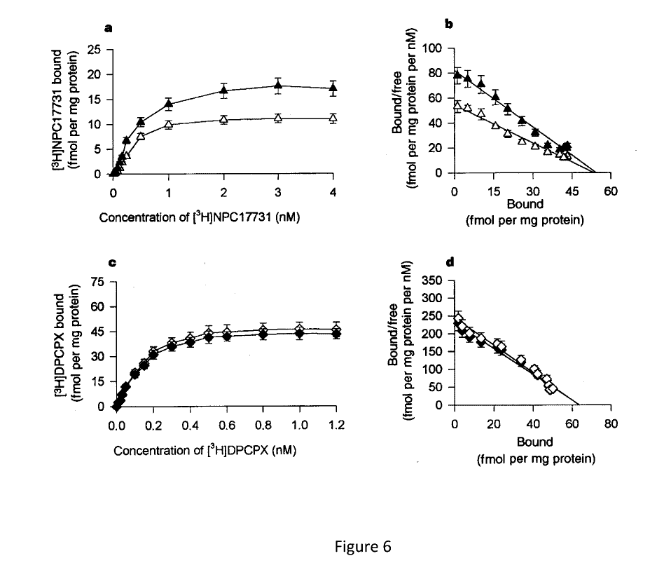

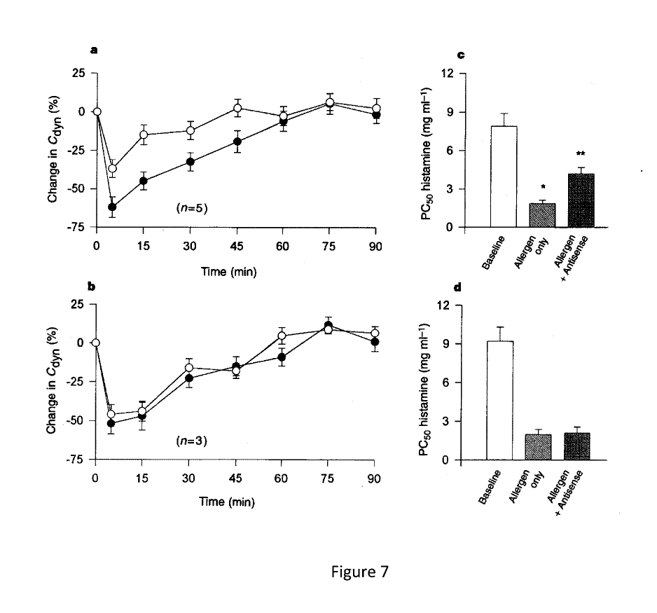

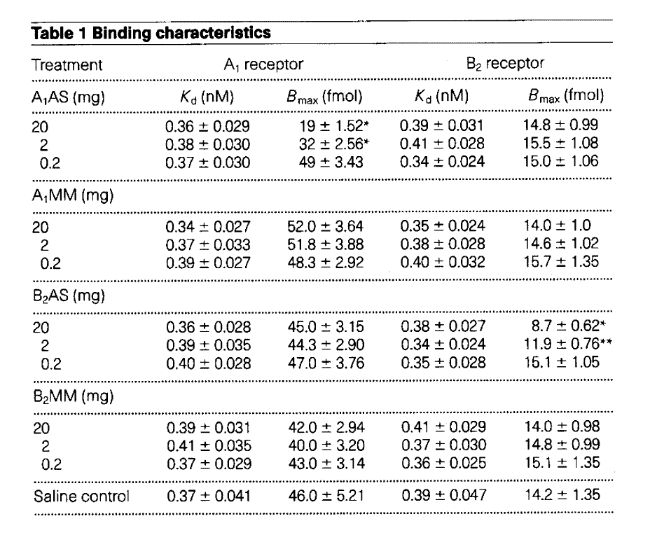

As an example of local administration of an antisense oligonucleotide, we utilized a phosphorothioate antisense oligonucleotide targeting the adenosine Al receptor delivered by inhalation to rabbits with well-quantitated bronchoconstriction upon challenge with adenosine (Nyce and Metzger, Nature 385:721-725, 1997). The antisense oligonucleotide targeted the same region of the target mRNA, centering on the initiation codon, as all of our selected antisense oligonucleotides in the sequence listing. FIGS. 4-7 and Table 1 show results of studies which show selective and potent inhibition of the adenosine Al receptor, and inhibition of adenosine Al receptor-mediated bronchoconstriction in these animals. The lung is an excellent local target for aerosolized antisense oligonucleotides because it is lined with surfactant, a material that appears to facilitate the pulmonary distribution and intracellular uptake of respired oligonucleotides.

In separate studies we showed that adenosine Al receptors in other tissues outside the lung were unaffected by the adenosine Al receptor antisense applied to the lung by inhalation.

These studies show one mechanism by which antisense oligonucleotides can be applied locally to an organ or tissue, for example a dog with lung cancer, avoiding systemic toxicity.

While the antisense oligonucleotide is applied locally, here via inhalation, the PPP inhibitor can be applied systemically where that is of advantage. For example, in neutered dogs with cancer, where DHEA is the PPP inhibitor used, DHEA has the added advantage of replenishing steroid hormone levels in the dog via Extra Gonadal Steroid Synthesis (EGSS). We have shown that EGSS can dramatically improve the quality of life in neutered dogs, both those with cancer and those without.

Inhalation is not the only method by which local delivery of antisense oligonucleotides can be made. In a separate series of dogs we inoculated tumors directly with antisense oligonucleotides drawn from our sequence list, and delivered DHEA as the PPP inhibitor either orally or by transdermal application. Results were positive. In at least 50% of treated dogs, with a variety of different tumor types, we observed what appeared to be complete tumor regression.

Current thinking about cancer suggests that tumors are distinguished by their genetic and epigenetic signatures, that is, both the mutations (genetic) and the changes in gene expression (epigenetic) that have occurred within the tumor cell population. It is likely that many drivers, both genetic and epigenetic, are involved in each neoplasm, whether in a dog or human. The present invention provides a unique method to approach a large number of targets simultaneously while confining most toxicity to the tumor volume itself.

### United States Patents Cited

U.S. Pat. No. 5,641,754 A Iversen, P L, 1997 Antisense oligonucleotide compositions for selectively killing cancer cells
U.S. Pat. No. 5,691,317 A, Yoon, S C C, 1997, Antisense oligonucleotides of human regulatory subunit RI-alpha of cAMP dependent protein kinases for the treatment of cancer.
U.S. Pat. No. 5,801,154 A, Baracchini, E et al, 1998, Antisense oligonucleotide modulation of multidrug resistance-associated protein
U.S. Pat. No. 5,994,315 A, Nyce, J W and Metzger, J W, 1999, Low adenosine agent, composition, kit and method for treatment of airway disease
U.S. Pat. No. 6,825,174 B2, Nyce, J W, 2004, Composition, formulations & method for prevention & treatment of diseases associated with bronchoconstriction, allergy(ies) & inflammation
U.S. Pat. No. 6,040,296 A, Nyce, J W, 2000, Specific antisense oligonucleotide composition & method for treatment of disorders associated with bronchoconstriction and lung inflammation
U.S. Pat. No. 6,025,339 A, Nyce, J W, 2000, Composition, kit and method for treatment of disorders associated with bronchoconstriction and lung inflammation
U.S. Pat. No. 6,670,349 B1, Nyce, J W, 2003, Composition & method for altering levels of or sensitivity to adenosine with a dehydroepiandrosterone &/or a ubiquinone
U.S. Pat. No. 7,456,161 B2, Nyce, J W, 2008, Use of DHEA and DHEA-sulfate for the treatment of chronic obstructive pulmonary disease
U.S. Pat. No. 6,432,924 B1, Nyce, J W, 2002, Method of treating disorders characterized by overexpression of cytidine deaminase or deoxycytidine deaminase overexpression
U.S. Pat. No. 6,136,791, Nyce, J W, 2000, Agent and method for treating disorders associated with cytidine deaminase or deoxycytidine deaminase overexpression
U.S. Pat. No. 6,087,351, Nyce, J W, 2000, Method for reducing adenosine levels with a Dehydroepiandrosterone and optionally a ubiquinone
U.S. Pat. No. 5,660,835, Nyce, J W, 1997, Method of treating adenosine depletion.
U.S. Pat. No. 5,527,789, Nyce, J W, 1996, Method of inhibiting carcinogenesis by treatment with Dehydroepiandrosterone and analogs thereof
U.S. Pat. No. 6,017,898 A, Zbigniew Pietrzkowski, Dariusz Cieslak, Gordana Olbina, 2000, Inhibition of tumor growth by antisense oligonucleotides for IL-8 and IL-8 receptor
U.S. Pat. No. 6,727,230 B1, Stephen L. Hutcherson, Josephine M. Glover, 2004, Immune stimulation by phosphorothioate oligonucleotide analogs
U.S. Pat. No. 7,001,890 B1, Wagner, H et al, 2006, Pharmaceutical compositions comprising a polynucleotide and optionally an antigen especially for vaccination
U.S. Pat. No. 7,402,574 B2, Iverson, P L et al, 2008, Antisense composition and method for treating cancer.
U.S. Pat. No. 7,569,551 B2 Gleave, N I et al, 2009, Chemo and radiation sensitization of cancer by antisense TRPM-2 oligodeoxynuclectides
U.S. Pat. No. 7,585,968 B2, James G. Karras, Kenneth W. Dobie , 2009, Compositions and their uses directed to thymus and activation-regulated chemokine (TARC)
U.S. Pat. No. 7,704,968 B2, Nerenberg, M I, 2010, Suppression of nuclear factor-KB dependent processes using oligonucleotides
U.S. Pat. No. 8,119,790 B2, Paolo Renzi, Khalid Zemzoumi 2012, Antisense oligonucleotides for treating allergy and neoplastic cell proliferation
U.S. Pat. No. 8,710,020 B2, Gleave, M E and Cormack, S D, 2014, Clusterin antisense therapy for treatment of cancer
U.S. Pat. No. 8,951,527 B2, Isenberg, J S et al, 2015, Radioprotectants targeting thrombospondin-1 and CD47

### Foreign Patents and Applications Cited

EP1009822 A1, Higenbottam, T, 2000, Antisense treatment of pulmonary hypertension WO 2012170250 Moulton, J 2011, Morpholino oligonucleotides capable of inhibiting cd47-mediated cellular damage and uses thereof. (Abstract: The present invention relates to morpholino oligonucleotides that exhibit high-affinity and high specificity for binding to CD47, and which have the ability to inhibit the translation of CD47. The invention further relates to the use of such morpholino oligonucleotides in radioprotection, and in the treatment of cancer, heart disease, CNS disorders and inflammation).
EP 1009822 A1 Higginbottom, T, 2000, Antisense treatment of pulmonary hypertension. (Abstract: The invention herein described relates to a method to treat pulmonary hypertension by antisense therapy using ET-1 derived antisense molecules delivered to the lungs as a pulse/spike in an inhaler).
WO2014153209 A1, Eriz, L O et al, 2014, (Abstract: The invention provides oligonucleotides complementary to a non-coding chimeric mitochondrial RNA as well as compositions and kits comprising the same, and their use in treating and preventing metastasis or relapse of a cancer in an individual previously treated for cancer with a therapy. The invention also provides oligonucleotides complementary to a non-coding chimeric mitochondrial RNA as well as compositions and kits comprising the same, and their use in treating a refractory cancer (e.g., a refractory HPV-associated cancer).

WO 1997011170 A1, Zamecnik, P A, 1997, Antisense oligonucleotide chemotherapy for benign hyperplasia or cancer of the prostate. (Abstract: Methods of selectively inhibiting the growth of or killing prostatic cells, using antisense oligonucleotides to prostate specific genes, are disclosed. The oligonucleotides may have natural nucleic acid structures or may be modified oligonucleotides with enhanced stability or tissue specific targeting. The prostate specific genes to which the antisense may be directed include the AR and the aFGF gene. Pharmaceutical compositions including such antisense oligonucleotides are also described for use in the methods. The methods and products are of particular utility in the treatment of benign prostatic hyperplasia or prostate cancer.

WO2001025422 A2, Bartelmez, S H and Iversen, P L, 2001, Antisense compositions and cancer-treatment methods. (Abstract: A composition comprising an antisense oligomer directed to an mRNA preferentially expressed in stem cells is described together with methods for treating stem cells with such a composition to increase the number of lineage committed progenitor cells and their progeny, and/or slow the growth of cancer cells. Also described is the use of such compositions and antisense oligonucleotide-treated stem cells as a medicament).
WO2008058225 A2, Brown, B D, 2008, Methods and compositions for treating cancer using bcl-2 antisense oligomers, tyrosine kinase inhibitors, and chemotherapeutic agents. (Abstract: Methods and compositions are provided for treating cell-proliferative related disorders such as cancer. Methods of inhibiting the growth of cancer cells comprise contacting the cancer cells with a Bcl-2 antisense oligomer; contacting the cancer cells with a tyrosine kinase inhibitor; and contacting the cancer cells with a cytotoxic chemotherapeutic agent. Methods of treating cancer in a human comprise administering to the human a Bcl-2 antisense oligomer, a tyrosine kinase inhibitor, and a cytotoxic chemotherapeutic agent. Kits containing compositions in amounts sufficient for at least one cycle of treatment comprise a triplet combination therapy of a Bcl-2 antisense oligomer, a tyrosine kinase inhibitor, and a cytotoxic chemotherapeutic agent. In selected embodiments, the tyrosine kinase inhibitor is one that targets cell surface kinase receptors, such as VEGFR (e.g., VEGFR1, VEGFR2, VEGFR3), PDGFR, KIT, and FLT-3).
CN1120170 C, [symbol] , 2003, Antisense thio-oligonucleotide of targeting C-raf cancer gene, medicinal composition containing same, and application in treating cancers. (Abstract: The present invention relates to targeting of c-raf oncogene antisense oligonucleotides, containing their use and pharmaceutical compositions for the treatment of cancer.
CA2471967 A1, Lopez-Bernstein et al, 2003, Wt1 antisense oligos for the inhibition of breast cancer. (Abstract: The present invention provides methods for inhibiting the growth of breast cancer cells and methods for treating breast cancers expressing Wilms' Tumor 1 (WT1) gene product using a WT1 antisense oligonucleotide. It further provides methods of predicting breast cancer progression and methods for the screening of candidate substances for activity against breast cancer.

### Other Citations

Alegre, M M et al, Cancer and Clinical Oncology 2(1):159, 2013.
Byun, Y, Doctoral Dissertation, The Ohio State University, 2006, Thymidine kinase as a molecular target for the development of novel anticancer and antibiotic agents [electronic resource]/by Youngjoo Byun.
Caudle, K E, Clinical pharmacology and therapeutics 94 (6): 640-5, 2013.
Fishman, P, Handbook Exp Pharmacol 193:399-441, 2013; doi: 10.1007/978-3-540-89616-9_14
Flavin, R et al, Future Oncol 6(4):551-562, 2010.
Geary, R S et al Adv Drug Deliv Rev. 2015 Feb 7. pii: 50169-409X(15)00010-1. doi: 10.1016/baddr.2015.01.008
Kim, M P and Gallick, E, Clinical Cancer Res 14:2247, 2008.
Lin, H. L. et al, British J Nutrition 95:870-878, 2006.
Liu, T. P. et al, Anti-Cancer Drugs 26(2):139-147, 2015.
McClarty, G A et. al., Biochemical and Biophysical Research Communications 154(3):975-981, 1988.
Mukherjee , Siddhartha, The Emperor of All Maladies, a Biography of Cancer; Simon & Schuster, 2010.
Nyce, J and Metzger, J M, Nature 385:721-725, 1997.
Paletta-Silva, R, Int J Mol Sci 14(2):3683-3704, 2013.
Pirollo. K F et al, Pharmacol & Therapeutics 99(1):55-77, 2003.
Robert, M F, Nat Genet 33(1):61-5, 2003.
Saif, M W, Cancer Genomics Proteomics. 2013 March-April; 10(2):89-92.
Schultz, S and Nyce, J W, cancer Res 51:6563-6567, 1991.
Staub, M and Eriksson, S, Cancer Drug Discovery and Development, Peters, G J ed., Humana Press, Inc., Totowa, N.J.

Straarup, E M et al, Nucleic acids res 38(20):7100-7111, doi: 10.1093/nar/gkg457
Swierczynski, J et al, World J Gastroenterology 20(9):2279-2303, 2014.
Torres de la Riva, G et al, PLoS One, 2013, 8(2):e55937

The embodiments of the invention in which an exclusive property or privilege is claimed are defined as follows:

1. A method of treating cancer in dogs comprising depletion of NADP(H) by inhibition of the Pentose Phosphate Pathway (PPP), combined with one or more antisense oligonucleotides targeting mRNA coding for NADPH-dependent proteins.

2. A composition according to the method of claim 1 in which the PPP inhibitor is any one or more of DHEA, DHEA sulfate, DHEA sulfatide, 7-keto-DHEA, Fluasterone (or its brominated analog), 3-acetyl-7-oxo DHEA, DHEA acetate, the DHEA metabolites androstenediol or androstendione, or any salts of each.

3. A composition according to the method in claim 2 in which the impact of systemic NADPH depletion caused by DHEA or one of its congeners is ameliorated by reconstituting the depleted metabolites tetrahydrobiotperin, N-6-isopentenyladenosine, a nitric oxide donor such as potassium nitrate, folinic acid (or products of the folate pathway), ubiquinone+tocotrienols, and certain monoamine precursors and cofactors including L-DOPA, 5-Hydroxytryptophan, pyridoxine, SAMe, ascorbate, pantothenic acid, and zinc.

4. A composition according to the method of claim 1 in which the antisense oligonucleotides are chemically modified to avoid nuclease degradation.

5. A composition according to the method of claim 1 in which the PPP inhibitor is an antisense oligonucleotide targeting the canine G6PD mRNA and is composed of the sequence 5′-cccgccggct catttaacca g-3′, Sequence ID Number 720, or another sequence from 19-25 bases long taken from the same general region of the target mRNA, overlapping its initiation codon as disclosed in the sequence listing.

6. A composition according to the method of claim 1 comprising an antisense oligonucleotide targeting the canine PGD mRNA, said antisense oligonucleotide composed of the sequence 5′-cggcctcggc catggcggcg g-3′, Sequence ID Number 504, or another sequence from 19-25 bases long taken from the same general region of the target mRNA, overlapping its initiation codon as disclosed in the sequence listing.

7. A composition according to the method of claim 1 comprising an antisense oligonucleotide targeting the canine TK1 mRNA, said antisense oligonucleotide composed of the sequence 5′-gcaggttgat gcagctcatg g-3′, Sequence ID Number 283, or another sequence from 19-25 bases long taken from the same general region of the target mRNA, overlapping its initiation codon as disclosed in the sequence listing.

8. A composition according to the method of claim 1 comprising an antisense oligonucleotide targeting the canine TS mRNA, said antisense oligonucleotide composed of the sequence 5′-gggcgggcat ggcgcgggcg g-3′, Sequence ID Number 1119, or another sequence from 19-25 bases long taken from the same general region of the target mRNA, overlapping its initiation codon as disclosed in the sequence listing.

9. A composition according to the method of claim 1 comprising an antisense oligonucleotide targeting the canine DHFR mRNA, said antisense oligonucleotide composed of the sequence 5′-ggctgcggcc ccatttcatg t-3′, Sequence ID Number 327, or another sequence from 19-25 bases long taken from the same general region of the target mRNA, overlapping its initiation codon as disclosed in the sequence listing.

10. A composition according to the method of claim 1 comprising an antisense oligonucleotide targeting the canine RNRM1 mRNA, said antisense oligonucleotide composed of the sequence 5′-cgcttgatca cgtgcatcgc g-3′, Sequence ID Number 741, or another sequence from 19-25 bases long taken from the same general region of the target mRNA, overlapping its initiation codon as disclosed in the sequence listing.

11. A composition according to the method of claim 1 comprising an antisense oligonucleotide targeting the canine RNRM2 mRNA, said antisense oligonucleotide composed of the sequence 5′-cgcggacgga gagcatggcg g-3′, Sequence ID Number 792, or another sequence from 19-25 bases long taken from the same general region of the target mRNA, overlapping its initiation codon as disclosed in the sequence listing.

12. A composition according to the method of claim 1 comprising an antisense oligonucleotide targeting the canine RNRM3 mRNA, said antisense oligonucleotide composed of the sequence 5′-cctgtttata cattttccaa a-3′, Sequence ID Number 893, or another sequence from 19-25 bases long taken from the same general region of the target mRNA, overlapping its initiation codon as disclosed in the sequence listing.

13. A composition according to the method of claim 1 comprising an antisense oligonucleotide targeting the canine c-kit mRNA, said antisense oligonucleotide composed of the sequence 5′-gcgagcgcct ctcatcgcgg t-3′, Sequence ID Number 249, or another sequence from 19-25 bases long taken from the same general region of the target mRNA, overlapping its initiation codon as disclosed in the sequence listing.

14. A composition according to the method of claim 1 comprising an antisense oligonucleotide targeting the canine FASN mRNA, said antisense oligonucleotide composed of the sequence 5′-cctcctccat ggctgctctg c-3′, Sequence ID Number 1015, or another sequence from 19-25 bases long taken from the same general region of the target mRNA, overlapping its initiation codon as disclosed in the sequence listing.

15. A composition according to the method of claim 1 comprising an antisense oligonucleotide targeting the canine BCL2 mRNA, said antisense oligonucleotide composed of the sequence 5′-gcccagcgtg cgccatcctc c-3′, Sequence ID Number 571, or another sequence from 19-25 bases long taken from the same general region of the target mRNA, overlapping its initiation codon as disclosed in the sequence listing.

16. A composition according to the method of claim 1 comprising an antisense oligonucleotide targeting the canine EZH2 mRNA, said antisense oligonucleotide composed of the sequence 5′-ggcccatgat tattctgcgc c-3′, Sequence ID Number 672, or another sequence from 19-25 bases long taken from the same general region of the target mRNA, overlapping its initiation codon as disclosed in the sequence listing.

17. A composition according to the method of claim 1 comprising an antisense oligonucleotide targeting the canine DNMT1 mRNA, said antisense oligonucleotide composed of the sequence 5′-ggcccatgat tattctgcgc c-3′, Sequence ID Number 672, or another sequence from 19-25 bases long taken from the same general region of the target mRNA, overlapping its initiation codon as disclosed in the sequence listing.

18. A composition according to the method of claim 1 comprising an antisense oligonucleotide targeting the canine Farnesyl Transferase mRNA, said antisense oligonucleotde composed of the sequence 5′-cacgaactcc atgctggcgg c-3′, Sequence ID Number 684, or another sequence from 19-25 bases long taken from the same general region of the target mRNA, overlapping its initiation codon as disclosed in the sequence listing.

19. A composition according to the method of claim 1 comprising an antisense oligonucleotide targeting the canine GGT 1α mRNA, said antisense oligonucleotide composed of the sequence 5′-gcgcccgtgc atggtgccgg c-3′, Sequence ID Number 1077, or another sequence from 19-25 bases long taken from the same general region of the target mRNA, overlapping its initiation codon as disclosed in the sequence listing.

20. A composition according to the method of claim 1 comprising an antisense oligonucleotide targeting the canine GGT 1βmRNA, said antisense oligonucleotide composed of the sequence 5′-cgccgctcct ctacatcgaa c-3′, Sequence ID Number 1063, or another sequence from 19-25 bases long taken from the same general region of the target mRNA, overlapping its initiation codon as disclosed in the sequence listing.

21. A composition according to the method of claim 1 comprising an antisense oligonucleotide targeting the canine PRPS1 mRNA, said antisense oligonucleotide composed of the sequence 5′-cggcatcttg ggtgcctacc c-3′, Sequence ID Number 1046, or another sequence from 19-25 bases long taken from the same general region of the target mRNA, overlapping its initiation codon as disclosed in the sequence listing.

22. A composition according to the method of claim 1 comprising an antisense oligonucleotide targeting the canine PRPS2 mRNA, said antisense oligonucelotide composed of the sequence 5′-ccgatgacat ccttctccga t-3′, Sequence ID Number 1039, or another sequence from 19-25 bases long taken from the same general region of the target mRNA, overlapping its initiation codon as disclosed in the sequence listing.

23. A composition according to the method of claim 1 comprising an antisense oligonucleotide targeting the canine APRT mRNA, said antisense oligonucleotide composed of the sequence 5′-cctccagctc catgtcgctg cc-3′, Sequence ID Number 1129, or another sequence from 19-25 bases long taken from the same general region of the target mRNA, overlapping its initiation codon as disclosed in the sequence listing.

24. A composition according to the method of claim 1 comprising an antisense oligonucleotide targeting the canine IMPD1 mRNA, said antisense oligonucleotide composed of the sequence 5′-gcggcccctc catgcggagg c-3′, Sequence ID Number 1135, or another sequence from 19-25 bases long taken from the same general region of the target mRNA, overlapping its initiation codon as disclosed in the sequence listing.

25. A composition according to the method of claim 1 comprising an antisense oligonucleotide targeting the canine IMPD2 mRNA, said antisense oligonucletide composed of the sequence 5′-cgcatgcgca aagcgcgccg t-3′, Sequence ID Number 1160, or another sequence from 19-25 bases long taken from the same general region of the target mRNA, overlapping its initiation codon as disclosed in the sequence listing.

26. A composition according to the method of claim 1 comprising an antisense oligonucleotide targeting the canine Pol a mRNA, said antisense oligonucleotide composed of the sequence 5′-cgccgtctac cagcgccatg g-3′, Sequence ID Number 1023, or another sequence from 19-25 bases long as disclosed in the sequence listing.

27. A composition according to the method of claim 1 in which the combination of antisense oligonucleotides selected to treat a particular canine tumor is identified by transcriptome analysis of the tumor.

28. A composition according to the method of claim 1 in which the adenosines may be substituted for with 6-methyladenosine.

29. A composition according to the method of claim 1 in which 5′-CG-3′ dinucleotide motifs may be substituted for with 5′-5methylCG-3′ dinucleotide sequence motifs.

30. A composition according to the method of claim 1 in which the antisense oligonucleotides are selected so that they will not hybridize with 100% accuracy to their human mRNA sequence homologs, in order to prevent toxicity to humans during development or clinical use.


SEQUENCE LISTING (1223 sequences)

SEQ ID NO: 1 - DNA, 26 bp, Canis familiaris
ccccatgcccggagcgccagcccgcc

SEQ ID NO: 2 - DNA, 25 bp, Canis familiaris
ccccatgcccggagcgccagcccgc

SEQ ID NO: 3 - DNA, 24 bp, Canis familiaris
ccccatgcccggagcgccagcccg

SEQ ID NO: 4 - DNA, 23 bp, Canis familiaris
ccccatgcccggagcgccagccc

SEQ ID NO: 5 - DNA, 22 bp, Canis familiaris
ccccatgcccggagcgccagcc

SEQ ID NO: 6 - DNA, 21 bp, Canis familiaris
ccccatgcccggagcgccagc

SEQ ID NO: 7 - DNA, 20 bp, Canis familiaris
ccccatgcccggagcgccag

SEQ ID NO: 8 - DNA, 26 bp, Canis familiaris
cccgccggctccccatgcccggagcg

SEQ ID NO: 9 - DNA, 25 bp, Canis familiaris
ccgccggctccccatgcccggagcg

SEQ ID NO: 10 - DNA, 24 bp, Canis familiaris
cgccggctccccatgcccggagcg

SEQ ID NO: 11 - DNA, 23 bp, Canis familiaris
gccggctccccatgcccggagcg

SEQ ID NO: 12 - DNA, 22 bp, Canis familiaris
ccggctccccatgcccggagcg

SEQ ID NO: 13 - DNA, 21 bp, Canis familiaris
cggctccccatgcccggagcg

SEQ ID NO: 14 - DNA, 20 bp, Canis familiaris
ggctccccatgcccggagcg

SEQ ID NO: 15 - DNA, 21 bp, Canis familiaris
ctcatcgttctgaagtgacct

SEQ ID NO: 16 - DNA, 22 bp, Canis familiaris
ctcatcgttctgaagtgacctg

SEQ ID NO: 17 - DNA, 23 bp, Canis familiaris
ctcatcgttctgaagtgacctgc

SEQ ID NO: 18 - DNA, 24 bp, Canis familiaris
ctcatcgttctgaagtgacctgct

SEQ ID NO: 19 - DNA, 25 bp, Canis familiaris
ctcatcgttctgaagtgacctgctg

SEQ ID NO: 20 - DNA, 26 bp, Canis familiaris
ctcatcgttctgaagtgacctgctgg

SEQ ID NO: 21 - DNA, 27 bp, Canis familiaris
ctcatcgttctgaagtgacctgctggc

SEQ ID NO: 22 - DNA, 21 bp, Canis familiaris
ctgaagtgacctgctggcggg

SEQ ID NO: 23 - DNA, 22 bp, Canis familiaris
tctgaagtgacctgctggcggg

SEQ ID NO: 24 - DNA, 23 bp, Canis familiaris
ttctgaagtgacctgctggcggg

SEQ ID NO: 25 - DNA, 24 bp, Canis familiaris
gttctgaagtgacctgctggcggg

SEQ ID NO: 26 - DNA, 25 bp, Canis familiaris
cgttctgaagtgacctgctggcggg

SEQ ID NO: 27 - DNA, 26 bp, Canis familiaris
tcgttctgaagtgacctgctggcggg

SEQ ID NO: 28 - DNA, 25 bp, Canis familiaris
ggcctcctagcatagcccatctccg

SEQ ID NO: 29 - DNA, 24 bp, Canis familiaris
ggcctcctagcatagcccatctcc

SEQ ID NO: 30 - DNA, 23 bp, Canis familiaris
ggcctcctagcatagcccatctc

SEQ ID NO: 31 - DNA, 22 bp, Canis familiaris
ggcctcctagcatagcccatct

SEQ ID NO: 32 - DNA, 21 bp, Canis familiaris
ggcctcctagcatagcccatc

SEQ ID NO: 33 - DNA, 25 bp, Canis familiaris
gcctcctagcatagcccatctccgc

SEQ ID NO: 34 - DNA, 24 bp, Canis familiaris
cctcctagcatagcccatctccgc

SEQ ID NO: 35 - DNA, 23 bp, Canis familiaris
ctcctagcatagcccatctccgc

SEQ ID NO: 36 - DNA, 22 bp, Canis familiaris
tcctagcatagcccatctccgc

SEQ ID NO: 37 - DNA, 21 bp, Canis familiaris
cctagcatagcccatctccgc

SEQ ID NO: 38 - DNA, 25 bp, Canis familiaris
cctcctagcatagcccatctccgct

SEQ ID NO: 39 - DNA, 24 bp, Canis familiaris
ctcctagcatagcccatctccgct

SEQ ID NO: 40 - DNA, 23 bp, Canis familiaris
tcctagcatagcccatctccgct

SEQ ID NO: 41 - DNA, 22 bp, Canis familiaris
cctagcatagcccatctccgct

SEQ ID NO: 42 - DNA, 21 bp, Canis familiaris
ctagcatagcccatctccgct

SEQ ID NO: 43 - DNA, 25 bp, Canis familiaris
ctcctagcatagcccatctccgcta

SEQ ID NO: 44 - DNA, 24 bp, Canis familiaris
tcctagcatagcccatctccgcta

SEQ ID NO: 45 - DNA, 23 bp, Canis familiaris
cctagcatagcccatctccgcta

SEQ ID NO: 46 - DNA, 22 bp, Canis familiaris
ctagcatagcccatctccgcta

SEQ ID NO: 47 - DNA, 21 bp, Canis familiaris
tagcatagcccatctccgcta

SEQ ID NO: 48 - DNA, 25 bp, Canis familiaris
tcctagcatagcccatctccgctag

SEQ ID NO: 49 - DNA, 24 bp, Canis familiaris
cctagcatagcccatctccgctag

SEQ ID NO: 50 - DNA, 23 bp, Canis familiaris
ctagcatagcccatctccgctag

SEQ ID NO: 51 - DNA, 22 bp, Canis familiaris
tagcatagcccatctccgctag

SEQ ID NO: 52 - DNA, 21 bp, Canis familiaris
agcatagcccatctccgctag

SEQ ID NO: 53 - DNA, 25 bp, Canis familiaris
cctagcatagcccatctccgctagg

SEQ ID NO: 54 - DNA, 24 bp, Canis familiaris
ctagcatagcccatctccgctagg

SEQ ID NO: 55 - DNA, 23 bp, Canis familiaris
tagcatagcccatctccgctagg

SEQ ID NO: 56 - DNA, 22 bp, Canis familiaris
agcatagcccatctccgctagg

SEQ ID NO: 57 - DNA, 21 bp, Canis familiaris
gcatagcccatctccgctagg

SEQ ID NO: 58 - DNA, 25 bp, Canis familiaris
ctagcatagcccatctccgctaggg

SEQ ID NO: 59 - DNA, 24 bp, Canis familiaris
tagcatagcccatctccgctaggg

SEQ ID NO: 60 - DNA, 23 bp, Canis familiaris
agcatagcccatctccgctaggg

SEQ ID NO: 61 - DNA, 22 bp, Canis familiaris
gcatagcccatctccgctaggg

SEQ ID NO: 62 - DNA, 21 bp, Canis familiaris
catagcccatctccgctaggg

SEQ ID NO: 63 - DNA, 26 bp, Canis familiaris
cagccatgatacaggagaagccaagg

SEQ ID NO: 64 - DNA, 25 bp, Canis familiaris
cagccatgatacaggagaagccaag

SEQ ID NO: 65 - DNA, 24 bp, Canis familiaris
cagccatgatacaggagaagccaa

SEQ ID NO: 66 - DNA, 23 bp, Canis familiaris
cagccatgatacaggagaagcca

SEQ ID NO: 67 - DNA, 22 bp, Canis familiaris
cagccatgatacaggagaagcc

SEQ ID NO: 68 - DNA, 21 bp, Canis familiaris
cagccatgatacaggagaagc

SEQ ID NO: 69 - DNA, 20 bp, Canis familiaris
cagccatgatacaggagaag

SEQ ID NO: 70 - DNA, 25 bp, Canis familiaris
agccatgatacaggagaagccaagg

SEQ ID NO: 71 - DNA, 24 bp, Canis familiaris
gccatgatacaggagaagccaagg

SEQ ID NO: 72 - DNA, 23 bp, Canis familiaris
ccatgatacaggagaagccaagg

SEQ ID NO: 73 - DNA, 22 bp, Canis familiaris
catgatacaggagaagccaagg

SEQ ID NO: 74 - DNA, 21 bp, Canis familiaris
atgatacaggagaagccaagg

SEQ ID NO: 75 - DNA, 20 bp, Canis familiaris
tgatacaggagaagccaagg

SEQ ID NO: 76 - DNA, 25 bp, Canis familiaris
cacccaggagcccatggtggacatg

SEQ ID NO: 77 - DNA, 24 bp, Canis familiaris
cacccaggagcccatggtggacat

SEQ ID NO: 78 - DNA, 23 bp, Canis familiaris
cacccaggagcccatggtggaca

SEQ ID NO: 79 - DNA, 22 bp, Canis familiaris
cacccaggagcccatggtggac

SEQ ID NO: 80 - DNA, 21 bp, Canis familiaris
cacccaggagcccatggtgga

SEQ ID NO: 81 - DNA, 20 bp, Canis familiaris
cacccaggagcccatggtgg

SEQ ID NO: 82 - DNA, 25 bp, Canis familiaris
acccaggagcccatggtggacatgg

SEQ ID NO: 83 - DNA, 24 bp, Canis familiaris
cccaggagcccatggtggacatgg

SEQ ID NO: 84 - DNA, 23 bp, Canis familiaris
ccaggagcccatggtggacatgg

SEQ ID NO: 85 - DNA, 22 bp, Canis familiaris
caggagcccatggtggacatgg

SEQ ID NO: 86 - DNA, 21 bp, Canis familiaris
aggagcccatggtggacatgg

SEQ ID NO: 87 - DNA, 20 bp, Canis familiaris
ggagcccatggtggacatgg

SEQ ID NO: 88 - DNA, 25 bp, Canis familiaris
cccaggagcccatggtggacatggc

SEQ ID NO: 89 - DNA, 24 bp, Canis familiaris
ccaggagcccatggtggacatggc

SEQ ID NO: 90 - DNA, 23 bp, Canis familiaris
caggagcccatggtggacatggc

SEQ ID NO: 91 - DNA, 22 bp, Canis familiaris
aggagcccatggtggacatggc

SEQ ID NO: 92 - DNA, 21 bp, Canis familiaris
ggagcccatggtggacatggc

SEQ ID NO: 93 - DNA, 25 bp, Canis familiaris
ccaggagcccatggtggacatggct

SEQ ID NO: 94 - DNA, 24 bp, Canis familiaris
caggagcccatggtggacatggct

SEQ ID NO: 95 - DNA, 23 bp, Canis familiaris
aggagcccatggtggacatggct

SEQ ID NO: 96 - DNA, 22 bp, Canis familiaris
ggagcccatggtggacatggct

SEQ ID NO: 97 - DNA, 21 bp, Canis familiaris
gagcccatggtggacatggct

SEQ ID NO: 98 - DNA, 25 bp, Canis familiaris
caggagcccatggtggacatggctg

SEQ ID NO: 99 - DNA, 24 bp, Canis familiaris
aggagcccatggtggacatggctg

SEQ ID NO: 100 - DNA, 23 bp, Canis familiaris
ggagcccatggtggacatggctg

SEQ ID NO: 101 - DNA, 22 bp, Canis familiaris
gagcccatggtggacatggctg

SEQ ID NO: 102 - DNA, 21 bp, Canis familiaris
agcccatggtggacatggctg

SEQ ID NO: 103 - DNA, 25 bp, Canis familiaris
aggagcccatggtggacatggctgc

SEQ ID NO: 104 - DNA, 24 bp, Canis familiaris
ggagcccatggtggacatggctgc

SEQ ID NO: 105 - DNA, 23 bp, Canis familiaris
gagcccatggtggacatggctgc

SEQ ID NO: 106 - DNA, 22 bp, Canis familiaris
agcccatggtggacatggctgc

SEQ ID NO: 107 - DNA, 21 bp, Canis familiaris
gcccatggtggacatggctgc

SEQ ID NO: 108 - DNA, 22 bp, Canis familiaris
atggctgcagacgggcagggtc

SEQ ID NO: 109 - DNA, 25 bp, Canis familiaris
gggccacatctccgcggccgccgcg

SEQ ID NO: 110 - DNA, 24 bp, Canis familiaris
gggccacatctccgcggccgccgc

SEQ ID NO: 111 - DNA, 23 bp, Canis familiaris
gggccacatctccgcggccgccg

SEQ ID NO: 112 - DNA, 22 bp, Canis familiaris
gggccacatctccgcggccgcc

SEQ ID NO: 113 - DNA, 21 bp, Canis familiaris
gggccacatctccgcggccgc

SEQ ID NO: 114 - DNA, 20 bp, Canis familiaris
gggccacatctccgcggccg

SEQ ID NO: 115 - DNA, 25 bp, Canis familiaris
ggccacatctccgcggccgccgcgg

SEQ ID NO: 116 - DNA, 24 bp, Canis familiaris
ggccacatctccgcggccgccgcg

SEQ ID NO: 117 - DNA, 23 bp, Canis familiaris
ggccacatctccgcggccgccgc

SEQ ID NO: 118 - DNA, 22 bp, Canis familiaris
ggccacatctccgcggccgccg

SEQ ID NO: 119 - DNA, 21 bp, Canis familiaris
ggccacatctccgcggccgcc

SEQ ID NO: 120 - DNA, 20 bp, Canis familiaris
ggccacatctccgcggccgc

SEQ ID NO: 121 - DNA, 25 bp, Canis familiaris
gccacatctccgcggccgccgcggg

SEQ ID NO: 122 - DNA, 24 bp, Canis familiaris
gccacatctccgcggccgccgcgg

SEQ ID NO: 123 - DNA, 23 bp, Canis familiaris
gccacatctccgcggccgccgcg

SEQ ID NO: 124 - DNA, 22 bp, Canis familiaris
gccacatctccgcggccgccgc

SEQ ID NO: 125 - DNA, 21 bp, Canis familiaris
gccacatctccgcggccgccg

SEQ ID NO: 126 - DNA, 20 bp, Canis familiaris
gccacatctccgcggccgcc

SEQ ID NO: 127 - DNA, 25 bp, Canis familiaris
ccggaatccaaagccagccatggct

SEQ ID NO: 128 - DNA, 24 bp, Canis familiaris
ccggaatccaaagccagccatggc

SEQ ID NO: 129 - DNA, 23 bp, Canis familiaris
ccggaatccaaagccagccatgg

SEQ ID NO: 130 - DNA, 22 bp, Canis familiaris
ccggaatccaaagccagccatg

SEQ ID NO: 131 - DNA, 21 bp, Canis familiaris
ccggaatccaaagccagccat

SEQ ID NO: 132 - DNA, 25 bp, Canis familiaris
cggaatccaaagccagccatggctt

SEQ ID NO: 133 - DNA, 24 bp, Canis familiaris
cggaatccaaagccagccatggct

SEQ ID NO: 134 - DNA, 23 bp, Canis familiaris
cggaatccaaagccagccatggc

SEQ ID NO: 135 - DNA, 22 bp, Canis familiaris
cggaatccaaagccagccatgg

SEQ ID NO: 136 - DNA, 21 bp, Canis familiaris
cggaatccaaagccagccatg

SEQ ID NO: 137 - DNA, 25 bp, Canis familiaris
ggaatccaaagccagccatggcttt

SEQ ID NO: 138 - DNA, 24 bp, Canis familiaris
ggaatccaaagccagccatggctt

SEQ ID NO: 139 - DNA, 23 bp, Canis familiaris
ggaatccaaagccagccatggct

SEQ ID NO: 140 - DNA, 22 bp, Canis familiaris
ggaatccaaagccagccatggc

SEQ ID NO: 141 - DNA, 21 bp, Canis familiaris
ggaatccaaagccagccatgg

SEQ ID NO: 142 - DNA, 25 bp, Canis familiaris
gaatccaaagccagccatggcttta

SEQ ID NO: 143 - DNA, 24 bp, Canis familiaris
gaatccaaagccagccatggcttt

SEQ ID NO: 144 - DNA, 23 bp, Canis familiaris
gaatccaaagccagccatggctt

SEQ ID NO: 145 - DNA, 22 bp, Canis familiaris
gaatccaaagccagccatggct

SEQ ID NO: 146 - DNA, 21 bp, Canis familiaris
gaatccaaagccagccatggc

SEQ ID NO: 147 - DNA, 20 bp, Canis familiaris
gaatccaaagccagccatgg

SEQ ID NO: 148 - DNA, 25 bp, Canis familiaris
aatccaaagccagccatggctttat

SEQ ID NO: 149 - DNA, 24 bp, Canis familiaris
aatccaaagccagccatggcttta

SEQ ID NO: 150 - DNA, 23 bp, Canis familiaris
aatccaaagccagccatggcttt

SEQ ID NO: 151 - DNA, 22 bp, Canis familiaris
aatccaaagccagccatggctt

SEQ ID NO: 152 - DNA, 21 bp, Canis familiaris
aatccaaagccagccatggct

SEQ ID NO: 153 - DNA, 20 bp, Canis familiaris
aatccaaagccagccatggc

SEQ ID NO: 154 - DNA, 25 bp, Canis familiaris
atccaaagccagccatggctttatg

SEQ ID NO: 155 - DNA, 24 bp, Canis familiaris
atccaaagccagccatggctttat

SEQ ID NO: 156 - DNA, 23 bp, Canis familiaris
atccaaagccagccatggcttta

SEQ ID NO: 157 - DNA, 22 bp, Canis familiaris
atccaaagccagccatggcttt

SEQ ID NO: 158 - DNA, 21 bp, Canis familiaris
atccaaagccagccatggctt

SEQ ID NO: 159 - DNA, 20 bp, Canis familiaris
atccaaagccagccatggct

SEQ ID NO: 160 - DNA, 25 bp, Canis familiaris
tccaaagccagccatggctttatgg

SEQ ID NO: 161 - DNA, 24 bp, Canis familiaris
tccaaagccagccatggctttatg

SEQ ID NO: 162 - DNA, 23 bp, Canis familiaris
tccaaagccagccatggctttat

SEQ ID NO: 163 - DNA, 22 bp, Canis familiaris
tccaaagccagccatggcttta

SEQ ID NO: 164 - DNA, 21 bp, Canis familiaris
tccaaagccagccatggcttt

SEQ ID NO: 165 - DNA, 20 bp, Canis familiaris
tccaaagccagccatggctt

SEQ ID NO: 166 - DNA, 25 bp, Canis familiaris
ccaaagccagccatggctttatgga

SEQ ID NO: 167 - DNA, 24 bp, Canis familiaris
ccaaagccagccatggctttatgg

SEQ ID NO: 168 - DNA, 23 bp, Canis familiaris
ccaaagccagccatggctttatg

SEQ ID NO: 169 - DNA, 22 bp, Canis familiaris
ccaaagccagccatggctttat

SEQ ID NO: 170 - DNA, 21 bp, Canis familiaris
ccaaagccagccatggcttta

SEQ ID NO: 171 - DNA, 20 bp, Canis familiaris
ccaaagccagccatggcttt

SEQ ID NO: 172 - DNA, 25 bp, Canis familiaris
caaagccagccatggctttatggag

SEQ ID NO: 173 - DNA, 24 bp, Canis familiaris
caaagccagccatggctttatgga

SEQ ID NO: 174 - DNA, 23 bp, Canis familiaris
caaagccagccatggctttatgg

SEQ ID NO: 175 - DNA, 22 bp, Canis familiaris
caaagccagccatggctttatg

SEQ ID NO: 176 - DNA, 21 bp, Canis familiaris
caaagccagccatggctttat

SEQ ID NO: 177 - DNA, 20 bp, Canis familiaris
caaagccagccatggcttta

SEQ ID NO: 178 - DNA, 25 bp, Canis familiaris
gccagccatggctttatggagcagt

SEQ ID NO: 179 - DNA, 24 bp, Canis familiaris
gccagccatggctttatggagcag

SEQ ID NO: 180 - DNA, 23 bp, Canis familiaris
gccagccatggctttatggagca

SEQ ID NO: 181 - DNA, 22 bp, Canis familiaris
gccagccatggctttatggagc

SEQ ID NO: 182 - DNA, 21 bp, Canis familiaris
gccagccatggctttatggag

SEQ ID NO: 183 - DNA, 20 bp, Canis familiaris
gccagccatggctttatgga

SEQ ID NO: 184 - DNA, 19 bp, Canis familiaris
gccagccatggctttatgg

SEQ ID NO: 185 - DNA, 25 bp, Canis familiaris
cccaggcgccgcgagcgcctctcat

SEQ ID NO: 186 - DNA, 24 bp, Canis familiaris
cccaggcgccgcgagcgcctctca

SEQ ID NO: 187 - DNA, 23 bp, Canis familiaris
cccaggcgccgcgagcgcctctc

SEQ ID NO: 188 - DNA, 22 bp, Canis familiaris
cccaggcgccgcgagcgcctct

SEQ ID NO: 189 - DNA, 21 bp, Canis familiaris
cccaggcgccgcgagcgcctc

SEQ ID NO: 190 - DNA, 20 bp, Canis familiaris
cccaggcgccgcgagcgcct

SEQ ID NO: 191 - DNA, 25 bp, Canis familiaris
ccaggcgccgcgagcgcctctcatc

SEQ ID NO: 192 - DNA, 24 bp, Canis familiaris
ccaggcgccgcgagcgcctctcat

SEQ ID NO: 193 - DNA, 23 bp, Canis familiaris
ccaggcgccgcgagcgcctctca

SEQ ID NO: 194 - DNA, 22 bp, Canis familiaris
ccaggcgccgcgagcgcctctc

SEQ ID NO: 195 - DNA, 21 bp, Canis familiaris
ccaggcgccgcgagcgcctct

SEQ ID NO: 196 - DNA, 20 bp, Canis familiaris
ccaggcgccgcgagcgcctc

SEQ ID NO: 197 - DNA, 25 bp, Canis familiaris
caggcgccgcgagcgcctctcatcg

SEQ ID NO: 198 - DNA, 24 bp, Canis familiaris
caggcgccgcgagcgcctctcatc

SEQ ID NO: 199 - DNA, 23 bp, Canis familiaris
caggcgccgcgagcgcctctcat

SEQ ID NO: 200 - DNA, 22 bp, Canis familiaris
caggcgccgcgagcgcctctca

SEQ ID NO: 201 - DNA, 21 bp, Canis familiaris
caggcgccgcgagcgcctctc

SEQ ID NO: 202 - DNA, 20 bp, Canis familiaris
caggcgccgcgagcgcctct

SEQ ID NO: 203 - DNA, 25 bp, Canis familiaris
aggcgccgcgagcgcctctcatcgc

SEQ ID NO: 204 - DNA, 24 bp, Canis familiaris
aggcgccgcgagcgcctctcatcg

SEQ ID NO: 205 - DNA, 23 bp, Canis familiaris
aggcgccgcgagcgcctctcatc

SEQ ID NO: 206 - DNA, 22 bp, Canis familiaris
aggcgccgcgagcgcctctcat

SEQ ID NO: 207 - DNA, 21 bp, Canis familiaris
aggcgccgcgagcgcctctca

SEQ ID NO: 208 - DNA, 20 bp, Canis familiaris
aggcgccgcgagcgcctctc

SEQ ID NO: 209 - DNA, 25 bp, Canis familiaris
ggcgccgcgagcgcctctcatcgcg

SEQ ID NO: 210 - DNA, 24 bp, Canis familiaris
ggcgccgcgagcgcctctcatcgc

SEQ ID NO: 211 - DNA, 23 bp, Canis familiaris
ggcgccgcgagcgcctctcatcg

SEQ ID NO: 212 - DNA, 22 bp, Canis familiaris
ggcgccgcgagcgcctctcatc

SEQ ID NO: 213 - DNA, 21 bp, Canis familiaris
ggcgccgcgagcgcctctcat

SEQ ID NO: 214 - DNA, 20 bp, Canis familiaris
ggcgccgcgagcgcctctca

SEQ ID NO: 215 - DNA, 25 bp, Canis familiaris
gcgccgcgagcgcctctcatcgcgg

SEQ ID NO: 216 - DNA, 24 bp, Canis familiaris
gcgccgcgagcgcctctcatcgcg

SEQ ID NO: 217 - DNA, 23 bp, Canis familiaris
gcgccgcgagcgcctctcatcgc

SEQ ID NO: 218 - DNA, 22 bp, Canis familiaris
gcgccgcgagcgcctctcatcg

SEQ ID NO: 219 - DNA, 21 bp, Canis familiaris
gcgccgcgagcgcctctcatc

SEQ ID NO: 220 - DNA, 20 bp, Canis familiaris
gcgccgcgagcgcctctcat

SEQ ID NO: 221 - DNA, 25 bp, Canis familiaris
cgccgcgagcgcctctcatcgcggt

SEQ ID NO: 222 - DNA, 24 bp, Canis familiaris
cgccgcgagcgcctctcatcgcgg

SEQ ID NO: 223 - DNA, 23 bp, Canis familiaris
cgccgcgagcgcctctcatcgcg

SEQ ID NO: 224 - DNA, 22 bp, Canis familiaris
cgccgcgagcgcctctcatcgc

SEQ ID NO: 225 - DNA, 21 bp, Canis familiaris
cgccgcgagcgcctctcatcg

SEQ ID NO: 226 - DNA, 20 bp, Canis familiaris
cgccgcgagcgcctctcatc

SEQ ID NO: 227 - DNA, 25 bp, Canis familiaris
gccgcgagcgcctctcatcgcggtg

SEQ ID NO: 228 - DNA, 24 bp, Canis familiaris
gccgcgagcgcctctcatcgcggt

SEQ ID NO: 229 - DNA, 23 bp, Canis familiaris
gccgcgagcgcctctcatcgcgg

SEQ ID NO: 230 - DNA, 22 bp, Canis familiaris
gccgcgagcgcctctcatcgcg

SEQ ID NO: 231 - DNA, 21 bp, Canis familiaris
gccgcgagcgcctctcatcgc

SEQ ID NO: 232 - DNA, 20 bp, Canis familiaris
gccgcgagcgcctctcatcg

SEQ ID NO: 233 - DNA, 25 bp, Canis familiaris
ccgcgagcgcctctcatcgcggtgg

SEQ ID NO: 234 - DNA, 24 bp, Canis familiaris
ccgcgagcgcctctcatcgcggtg

SEQ ID NO: 235 - DNA, 23 bp, Canis familiaris
ccgcgagcgcctctcatcgcggt

SEQ ID NO: 236 - DNA, 22 bp, Canis familiaris
ccgcgagcgcctctcatcgcgg

SEQ ID NO: 237 - DNA, 21 bp, Canis familiaris
ccgcgagcgcctctcatcgcg

SEQ ID NO: 238 - DNA, 20 bp, Canis familiaris
ccgcgagcgcctctcatcgc

SEQ ID NO: 239 - DNA, 25 bp, Canis familiaris
cgcgagcgcctctcatcgcggtggc

SEQ ID NO: 240 - DNA, 24 bp, Canis familiaris
cgcgagcgcctctcatcgcggtgg

SEQ ID NO: 241 - DNA, 23 bp, Canis familiaris
cgcgagcgcctctcatcgcggtg

SEQ ID NO: 242 - DNA, 22 bp, Canis familiaris
cgcgagcgcctctcatcgcggt

SEQ ID NO: 243 - DNA, 21 bp, Canis familiaris
cgcgagcgcctctcatcgcgg

SEQ ID NO: 244 - DNA, 20 bp, Canis familiaris
cgcgagcgcctctcatcgcg

SEQ ID NO: 245 - DNA, 25 bp, Canis familiaris
gcgagcgcctctcatcgcggtggct

SEQ ID NO: 246 - DNA, 24 bp, Canis familiaris
gcgagcgcctctcatcgcggtggc

SEQ ID NO: 247 - DNA, 23 bp, Canis familiaris
gcgagcgcctctcatcgcggtgg

SEQ ID NO: 248 - DNA, 22 bp, Canis familiaris
gcgagcgcctctcatcgcggtg

SEQ ID NO: 249 - DNA, 21 bp, Canis familiaris
gcgagcgcctctcatcgcggt

SEQ ID NO: 250 - DNA, 20 bp, Canis familiaris
gcgagcgcctctcatcgcgg

SEQ ID NO: 251 - DNA, 25 bp, Canis familiaris
cgagcgcctctcatcgcggtggctg

SEQ ID NO: 252 - DNA, 25 bp, Canis familiaris
cgagcgcctctcatcgcggtggctc

SEQ ID NO: 253 - DNA, 23 bp, Canis familiaris
cgagcgcctctcatcgcggtggc

SEQ ID NO: 254 - DNA, 22 bp, Canis familiaris
cgagcgcctctcatcgcggtgg

SEQ ID NO: 255 - DNA, 21 bp, Canis familiaris
cgagcgcctctcatcgcggtg

SEQ ID NO: 256 - DNA, 20 bp, Canis familiaris
cgagcgcctctcatcgcggt

SEQ ID NO: 257 - DNA, 25 bp, Canis familiaris
gagcgcctctcatcgcggtggctgc

SEQ ID NO: 258 - DNA, 24 bp, Canis familiaris
gagcgcctctcatcgcggtggctg

SEQ ID NO: 259 - DNA, 23 bp, Canis familiaris
gagcgcctctcatcgcggtggct

SEQ ID NO: 260 - DNA, 22 bp, Canis familiaris
gagcgcctctcatcgcggtggc

SEQ ID NO: 261 - DNA, 21 bp, Canis familiaris
gagcgcctctcatcgcggtgg

SEQ ID NO: 262 - DNA, 20 bp, Canis familiaris
gagcgcctctcatcgcggtg

SEQ ID NO: 263 - DNA, 15 bp, Canis familiaris
cgcctctcatcgcgg

SEQ ID NO: 264 - DNA, 18 bp, Canis familiaris
cgcctctcatcgcggtgg

SEQ ID NO: 265 - DNA, 20 bp, Canis familiaris
gcgcctctcatcgcggtggc

SEQ ID NO: 266 - DNA, 25 bp, Canis familiaris
gggcaggttgatgcagctcatggcg

SEQ ID NO: 267 - DNA, 24 bp, Canis familiaris
gggcaggttgatgcagctcatggc

SEQ ID NO: 268 - DNA, 23 bp, Canis familiaris
gggcaggttgatgcagctcatgg

SEQ ID NO: 269 - DNA, 22 bp, Canis familiaris
gggcaggttgatgcagctcatg

SEQ ID NO: 270 - DNA, 21 bp, Canis familiaris
gggcaggttgatgcagctcat

SEQ ID NO: 271 - DNA, 20 bp, Canis familiaris
gggcaggttgatgcagctca

SEQ ID NO: 272 - DNA, 25 bp, Canis familiaris
ggcaggttgatgcagctcatggcgc

SEQ ID NO: 273 - DNA, 24 bp, Canis familiaris
ggcaggttgatgcagctcatggcg

SEQ ID NO: 274 - DNA, 23 bp, Canis familiaris
ggcaggttgatgcagctcatggc

SEQ ID NO: 275 - DNA, 22 bp, Canis familiaris
ggcaggttgatgcagctcatgg

SEQ ID NO: 276 - DNA, 21 bp, Canis familiaris
ggcaggttgatgcagctcatg

SEQ ID NO: 277 - DNA, 20 bp, Canis familiaris
ggcaggttgatgcagctcat

SEQ ID NO: 278 - DNA, 19 bp, Canis familiaris
ggcaggttgatgcagctca

SEQ ID NO: 279 - DNA, 25 bp, Canis familiaris
gcaggttgatgcagctcatggcgcc

SEQ ID NO: 280 - DNA, 24 bp, Canis familiaris
gcaggttgatgcagctcatggcgc

SEQ ID NO: 281 - DNA, 23 bp, Canis familiaris
gcaggttgatgcagctcatggcg

SEQ ID NO: 282 - DNA, 22 bp, Canis familiaris
gcaggttgatgcagctcatggc

SEQ ID NO: 283 - DNA, 21 bp, Canis familiaris
gcaggttgatgcagctcatgg

SEQ ID NO: 284 - DNA, 20 bp, Canis familiaris
gcaggttgatgcagctcatg

SEQ ID NO: 285 - DNA, 25 bp, Canis familiaris
caggttgatgcagctcatggcgcct

SEQ ID NO: 286 - DNA, 24 bp, Canis familiaris
caggttgatgcagctcatggcgcc

SEQ ID NO: 287 - DNA, 23 bp, Canis familiaris
caggttgatgcagctcatggcgc

SEQ ID NO: 288 - DNA, 22 bp, Canis familiaris
caggttgatgcagctcatggcg

SEQ ID NO: 289 - DNA, 21 bp, Canis familiaris
caggttgatgcagctcatggc

SEQ ID NO: 290 - DNA, 20 bp, Canis familiaris
caggttgatgcagctcatgg

SEQ ID NO: 291 - DNA, 25 bp, Canis familiaris
aggttgatgcagctcatggcgcctg

SEQ ID NO: 292 - DNA, 24 bp, Canis familiaris
aggttgatgcagctcatggcgcct

SEQ ID NO: 293 - DNA, 23 bp, Canis familiaris
aggttgatgcagctcatggcgcc

SEQ ID NO: 294 - DNA, 22 bp, Canis familiaris
aggttgatgcagctcatggcgc

SEQ ID NO: 295 - DNA, 21 bp, Canis familiaris
aggttgatgcagctcatggcg

SEQ ID NO: 296 - DNA, 20 bp, Canis familiaris
aggttgatgcagctcatggc

SEQ ID NO: 297 - DNA, 25 bp, Canis familiaris
ggttgatgcagctcatggcgcctgc

SEQ ID NO: 298 - DNA, 24 bp, Canis familiaris
ggttgatgcagctcatggcgcctg

SEQ ID NO: 299 - DNA, 23 bp, Canis familiaris
ggttgatgcagctcatggcgcct

SEQ ID NO: 300 - DNA, 22 bp, Canis familiaris
ggttgatgcagctcatggcgcc

SEQ ID NO: 301 - DNA, 21 bp, Canis familiaris
ggttgatgcagctcatggcgc

SEQ ID NO: 302 - DNA, 20 bp, Canis familiaris
ggttgatgcagctcatggcg

SEQ ID NO: 303 - DNA, 25 bp, Canis familiaris
gttgatgcagctcatggcgcctgca

SEQ ID NO: 304 - DNA, 24 bp, Canis familiaris
gttgatgcagctcatggcgcctgc

SEQ ID NO: 305 - DNA, 23 bp, Canis familiaris
gttgatgcagctcatggcgcctg

SEQ ID NO: 306 - DNA, 22 bp, Canis familiaris
gttgatgcagctcatggcgcct

SEQ ID NO: 307 - DNA, 21 bp, Canis familiaris
gttgatgcagctcatggcgcc

SEQ ID NO: 308 - DNA, 20 bp, Canis familiaris
gttgatgcagctcatggcgc

SEQ ID NO: 309 - DNA, 25 bp, Canis familiaris
ttgatgcagctcatggcgcctgcac

SEQ ID NO: 310 - DNA, 24 bp, Canis familiaris
ttgatgcagctcatggcgcctgca

SEQ ID NO: 311 - DNA, 22 bp, Canis familiaris
ttgatgcagctcatggcgcctg

SEQ ID NO: 312 - DNA, 22 bp, Canis familiaris
ttgatgcagctcatggcgcctg

SEQ ID NO: 313 - DNA, 21 bp, Canis familiaris
ttgatgcagctcatggcgcct

SEQ ID NO: 314 - DNA, 20 bp, Canis familiaris
ttgatgcagctcatggcgcc

SEQ ID NO: 315 - DNA, 25 bp, Canis familiaris
tgatgcagctcatggcgcctgcacg

SEQ ID NO: 316 - DNA, 24 bp, Canis familiaris
tgatgcagctcatggcgcctgcac

SEQ ID NO: 317 - DNA, 23 bp, Canis familiaris
tgatgcagctcatggcgcctgca

SEQ ID NO: 318 - DNA, 22 bp, Canis familiaris
tgatgcagctcatggcgcctgc

SEQ ID NO: 319 - DNA, 21 bp, Canis familiaris
tgatgcagctcatggcgcctg

SEQ ID NO: 320 - DNA, 20 bp, Canis familiaris
tgatgcagctcatggcgcct

SEQ ID NO: 321 - DNA, 15 bp, Canis familiaris
gcagctcatggcgcc

SEQ ID NO: 322 - DNA, 21 bp, Canis familiaris
gcagctcatggcgcctgcacg

SEQ ID NO: 323 - DNA, 25 bp, Canis familiaris
ggctgcggccccatttcatgttatt

SEQ ID NO: 324 - DNA, 24 bp, Canis familiaris
ggctgcggccccatttcatgttat

SEQ ID NO: 325 - DNA, 23 bp, Canis familiaris
ggctgcggccccatttcatgtta

SEQ ID NO: 326 - DNA, 22 bp, Canis familiaris
ggctgcggccccatttcatgtt

SEQ ID NO: 327 - DNA, 21 bp, Canis familiaris
ggctgcggccccatttcatgt

SEQ ID NO: 328 - DNA, 20 bp, Canis familiaris
ggctgcggccccatttcatg

SEQ ID NO: 329 - DNA, 25 bp, Canis familiaris
gctgcggccccatttcatgttattg

SEQ ID NO: 330 - DNA, 24 bp, Canis familiaris
gctgcggccccatttcatgttatt

SEQ ID NO: 331 - DNA, 23 bp, Canis familiaris
gctgcggccccatttcatgttat

SEQ ID NO: 332 - DNA, 22 bp, Canis familiaris
gctgcggccccatttcatgtta

SEQ ID NO: 333 - DNA, 21 bp, Canis familiaris
gctgcggccccatttcatgtt

SEQ ID NO: 334 - DNA, 20 bp, Canis familiaris
gctgcggccccatttcatgt

SEQ ID NO: 335 - DNA, 25 bp, Canis familiaris
ctgcggccccatttcatgttattga

SEQ ID NO: 336 - DNA, 24 bp, Canis familiaris
ctgcggccccatttcatgttattg

SEQ ID NO: 337 - DNA, 23 bp, Canis familiaris
ctgcggccccatttcatgttatt

SEQ ID NO: 338 - DNA, 22 bp, Canis familiaris
ctgcggccccatttcatgttat

SEQ ID NO: 339 - DNA, 21 bp, Canis familiaris
ctgcggccccatttcatgtta

SEQ ID NO: 340 - DNA, 20 bp, Canis familiaris
ctgcggccccatttcatgtt

SEQ ID NO: 341 - DNA, 19 bp, Canis familiaris
ctgcggccccatttcatgt

SEQ ID NO: 342 - DNA, 25 bp, Canis familiaris
tgcggccccatttcatgttattgag

SEQ ID NO: 343 - DNA, 24 bp, Canis familiaris
tgcggccccatttcatgttattga

SEQ ID NO: 344 - DNA, 23 bp, Canis familiaris
tgcggccccatttcatgttattg

SEQ ID NO: 345 - DNA, 22 bp, Canis familiaris
tgcggccccatttcatgttatt

SEQ ID NO: 346 - DNA, 21 bp, Canis familiaris
tgcggccccatttcatgttat

SEQ ID NO: 347 - DNA, 20 bp, Canis familiaris
tgcggccccatttcatgtta

SEQ ID NO: 348 - DNA, 25 bp, Canis familiaris
gcggccccatttcatgttattgaga

SEQ ID NO: 349 - DNA, 24 bp, Canis familiaris
gcggccccatttcatgttattgag

SEQ ID NO: 350 - DNA, 23 bp, Canis familiaris
gcggccccatttcatgttattga

SEQ ID NO: 351 - DNA, 22 bp, Canis familiaris
gcggccccatttcatgttattg

SEQ ID NO: 352 - DNA, 21 bp, Canis familiaris
gcggccccatttcatgttatt

SEQ ID NO: 353 - DNA, 20 bp, Canis familiaris
gcggccccatttcatgttat

SEQ ID NO: 354 - DNA, 25 bp, Canis familiaris
cggccccatttcatgttattgagag

SEQ ID NO: 355 - DNA, 24 bp, Canis familiaris
cggccccatttcatgttattgaga

SEQ ID NO: 356 - DNA, 23 bp, Canis familiaris
cggccccatttcatgttattgag

SEQ ID NO: 357 - DNA, 22 bp, Canis familiaris
cggccccatttcatgttattga

SEQ ID NO: 358 - DNA, 21 bp, Canis familiaris
cggccccatttcatgttattg

SEQ ID NO: 359 - DNA, 20 bp, Canis familiaris
cggccccatttcatgttatt

SEQ ID NO: 360 - DNA, 25 bp, Canis familiaris
ggccccatttcatgttattgagagg

SEQ ID NO: 361 - DNA, 24 bp, Canis familiaris
ggccccatttcatgttattgagag

SEQ ID NO: 362 - DNA, 23 bp, Canis familiaris
ggccccatttcatgttattgaga

SEQ ID NO: 363 - DNA, 22 bp, Canis familiaris
ggccccatttcatgttattgag

SEQ ID NO: 364 - DNA, 21 bp, Canis familiaris
ggccccatttcatgttattga

SEQ ID NO: 365 - DNA, 20 bp, Canis familiaris
ggccccatttcatgttattg

SEQ ID NO: 366 - DNA, 26 bp, Canis familiaris
ggccccatttcatgttattgagaggc

SEQ ID NO: 367 - DNA, 25 bp, Canis familiaris
ggccccatttcatgttattgagagg

SEQ ID NO: 368 - DNA, 24 bp, Canis familiaris
ggccccatttcatgttattgagag

SEQ ID NO: 369 - DNA, 23 bp, Canis familiaris
ggccccatttcatgttattgaga

SEQ ID NO: 370 - DNA, 22 bp, Canis familiaris
ggccccatttcatgttattgag

SEQ ID NO: 371 - DNA, 21 bp, Canis familiaris
ggccccatttcatgttattga

SEQ ID NO: 372 - DNA, 20 bp, Canis familiaris
ggccccatttcatgttattg

SEQ ID NO: 373 - DNA, 25 bp, Canis familiaris
gccccccaggtagtggccattctct

SEQ ID NO: 374 - DNA, 24 bp, Canis familiaris
gccccccaggtagtggccattctc

SEQ ID NO: 375 - DNA, 23 bp, Canis familiaris
gccccccaggtagtggccattct

SEQ ID NO: 376 - DNA, 22 bp, Canis familiaris
gccccccaggtagtggccattc

SEQ ID NO: 377 - DNA, 21 bp, Canis familiaris
gccccccaggtagtggccatt

SEQ ID NO: 378 - DNA, 20 bp, Canis familiaris
gccccccaggtagtggccat

SEQ ID NO: 379 - DNA, 25 bp, Canis familiaris
ccccccaggtagtggccattctctt

SEQ ID NO: 380 - DNA, 24 bp, Canis familiaris
ccccccaggtagtggccattctct

SEQ ID NO: 381 - DNA, 23 bp, Canis familiaris
ccccccaggtagtggccattctc

SEQ ID NO: 382 - DNA, 22 bp, Canis familiaris
ccccccaggtagtggccattct

SEQ ID NO: 383 - DNA, 21 bp, Canis familiaris
ccccccaggtagtggccattc

SEQ ID NO: 384 - DNA, 20 bp, Canis familiaris
ccccccaggtagtggccatt

SEQ ID NO: 385 - DNA, 19 bp, Canis familiaris
ccccccaggtagtggccat

SEQ ID NO: 386 - DNA, 25 bp, Canis familiaris
cccccaggtagtggccattctcttc

SEQ ID NO: 387 - DNA, 24 bp, Canis familiaris
cccccaggtagtggccattctctt

SEQ ID NO: 388 - DNA, 23 bp, Canis familiaris
cccccaggtagtggccattctct

SEQ ID NO: 389 - DNA, 22 bp, Canis familiaris
cccccaggtagtggccattctc

SEQ ID NO: 390 - DNA, 21 bp, Canis familiaris
cccccaggtagtggccattct

SEQ ID NO: 391 - DNA, 20 bp, Canis familiaris
cccccaggtagtggccattc

SEQ ID NO: 392 - DNA, 19 bp, Canis familiaris
cccccaggtagtggccatt

SEQ ID NO: 393 - DNA, 18 bp, Canis familiaris
cccccaggtagtggccat

SEQ ID NO: 394 - DNA, 25 bp, Canis familiaris
ccccaggtagtggccattctcttcc

SEQ ID NO: 395 - DNA, 24 bp, Canis familiaris
ccccaggtagtggccattctcttc

SEQ ID NO: 396 - DNA, 23 bp, Canis familiaris
ccccaggtagtggccattctctt

SEQ ID NO: 397 - DNA, 22 bp, Canis familiaris
ccccaggtagtggccattctct

SEQ ID NO: 398 - DNA, 21 bp, Canis familiaris
ccccaggtagtggccattctc

SEQ ID NO: 399 - DNA, 20 bp, Canis familiaris
ccccaggtagtggccattct

SEQ ID NO: 400 - DNA, 25 bp, Canis familiaris
cccaggtagtggccattctcttccc

SEQ ID NO: 401 - DNA, 24 bp, Canis familiaris
cccaggtagtggccattctcttcc

SEQ ID NO: 402 - DNA, 23 bp, Canis familiaris
cccaggtagtggccattctcttc

SEQ ID NO: 403 - DNA, 22 bp, Canis familiaris
cccaggtagtggccattctctt

SEQ ID NO: 404 - DNA, 21 bp, Canis familiaris
cccaggtagtggccattctct

SEQ ID NO: 405 - DNA, 20 bp, Canis familiaris
cccaggtagtggccattctc

SEQ ID NO: 406 - DNA, 15 bp, Canis familiaris
ccccaggtagtggcc

SEQ ID NO: 407 - DNA, 16 bp, Canis familiaris
cccccaggtagtggcc

SEQ ID NO: 408 - DNA, 17 bp, Canis familiaris
ccccccaggtagtggcc

SEQ ID NO: 409 - DNA, 18 bp, Canis familiaris
gccccccaggtagtggcc

SEQ ID NO: 410 - DNA, 25 bp, Canis familiaris
ggccgagatggcgggcggcatggca

SEQ ID NO: 411 - DNA, 24 bp, Canis familiaris
ggccgagatggcgggcggcatggc

SEQ ID NO: 412 - DNA, 23 bp, Canis familiaris
ggccgagatggcgggcggcatgg

SEQ ID NO: 413 - DNA, 24 bp, Canis familiaris
gccgagatggcgggcggcatggca

SEQ ID NO: 414 - DNA, 23 bp, Canis familiaris
gccgagatggcgggcggcatggc

SEQ ID NO: 415 - DNA, 22 bp, Canis familiaris
gccgagatggcgggcggcatgg

SEQ ID NO: 416 - DNA, 21 bp, Canis familiaris
gccgagatggcgggcggcatg

SEQ ID NO: 417 - DNA, 20 bp, Canis familiaris
gccgagatggcgggcggcat

SEQ ID NO: 418 - DNA, 25 bp, Canis familiaris
ccgagatggcgggcggcatggcagg

SEQ ID NO: 419 - DNA, 24 bp, Canis familiaris
ccgagatggcgggcggcatggcag

SEQ ID NO: 420 - DNA, 23 bp, Canis familiaris
ccgagatggcgggcggcatggca

SEQ ID NO: 421 - DNA, 22 bp, Canis familiaris
ccgagatggcgggcggcatggc

SEQ ID NO: 422 - DNA, 21 bp, Canis familiaris
ccgagatggcgggcggcatgg

SEQ ID NO: 423 - DNA, 20 bp, Canis familiaris
ccgagatggcgggcggcatg

SEQ ID NO: 424 - DNA, 25 bp, Canis familiaris
cgagatggcgggcggcatggcaggc

SEQ ID NO: 425 - DNA, 24 bp, Canis familiaris
cgagatggcgggcggcatggcagg

SEQ ID NO: 426 - DNA, 23 bp, Canis familiaris
cgagatggcgggcggcatggcag

SEQ ID NO: 427 - DNA, 22 bp, Canis familiaris
cgagatggcgggcggcatggca

SEQ ID NO: 428 - DNA, 21 bp, Canis familiaris
cgagatggcgggcggcatggc

SEQ ID NO: 429 - DNA, 20 bp, Canis familiaris
cgagatggcgggcggcatgg

SEQ ID NO: 430 - DNA, 22 bp, Canis familiaris
ggccgagatggcgggcggcatg

SEQ ID NO: 431 - DNA, 21 bp, Canis familiaris
ggccgagatggcgggcggcat

SEQ ID NO: 432 - DNA, 20 bp, Canis familiaris
ggccgagatggcgggcggca

SEQ ID NO: 433 - DNA, 25 bp, Canis familiaris
gccgagatggcgggcggcatggcag

SEQ ID NO: 434 - DNA, 25 bp, Canis familiaris
gagatggcgggcggcatggcaggcg

SEQ ID NO: 435 - DNA, 24 bp, Canis familiaris
gagatggcgggcggcatggcaggc

SEQ ID NO: 436 - DNA, 23 bp, Canis familiaris
gagatggcgggcggcatggcagg

SEQ ID NO: 437 - DNA, 22 bp, Canis familiaris
gagatggcgggcggcatggcag

SEQ ID NO: 438 - DNA, 21 bp, Canis familiaris
gagatggcgggcggcatggca

SEQ ID NO: 439 - DNA, 20 bp, Canis familiaris
gagatggcgggcggcatggc

SEQ ID NO: 440 - DNA, 25 bp, Canis familiaris
agatggcgggcggcatggcaggcgc

SEQ ID NO: 441 - DNA, 24 bp, Canis familiaris
agatggcgggcggcatggcaggcg

SEQ ID NO: 442 - DNA, 23 bp, Canis familiaris
agatggcgggcggcatggcaggc

SEQ ID NO: 443 - DNA, 22 bp, Canis familiaris
agatggcgggcggcatggcagg

SEQ ID NO: 444 - DNA, 21 bp, Canis familiaris
agatggcgggcggcatggcag

SEQ ID NO: 445 - DNA, 20 bp, Canis familiaris
agatggcgggcggcatggca

SEQ ID NO: 446 - DNA, 25 bp, Canis familiaris
gatggcgggcggcatggcaggcgcg

SEQ ID NO: 447 - DNA, 24 bp, Canis familiaris
gatggcgggcggcatggcaggcgc

SEQ ID NO: 448 - DNA, 23 bp, Canis familiaris
gatggcgggcggcatggcaggcg

SEQ ID NO: 449 - DNA, 22 bp, Canis familiaris
gatggcgggcggcatggcaggc

SEQ ID NO: 450 - DNA, 21 bp, Canis familiaris
gatggcgggcggcatggcagg

SEQ ID NO: 451 - DNA, 20 bp, Canis familiaris
gatggcgggcggcatggcag

SEQ ID NO: 452 - DNA, 25 bp, Canis familiaris
atggcgggcggcatggcaggcgcgg

SEQ ID NO: 453 - DNA, 24 bp, Canis familiaris
atggcgggcggcatggcaggcgcg

SEQ ID NO: 454 - DNA, 23 bp, Canis familiaris
atggcgggcggcatggcaggcgc

SEQ ID NO: 455 - DNA, 22 bp, Canis familiaris
atggcgggcggcatggcaggcg

SEQ ID NO: 456 - DNA, 21 bp, Canis familiaris
atggcgggcggcatggcaggc

SEQ ID NO: 457 - DNA, 20 bp, Canis familiaris
atggcgggcggcatggcagg

SEQ ID NO: 458 - DNA, 25 bp, Canis familiaris
tggcgggcggcatggcaggcgcggg

SEQ ID NO: 459 - DNA, 24 bp, Canis familiaris
tggcgggcggcatggcaggcgcgg

SEQ ID NO: 460 - DNA, 23 bp, Canis familiaris
tggcgggcggcatggcaggcgcg

SEQ ID NO: 461 - DNA, 22 bp, Canis familiaris
tggcgggcggcatggcaggcgc

SEQ ID NO: 462 - DNA, 21 bp, Canis familiaris
tggcgggcggcatggcaggcg

SEQ ID NO: 463 - DNA, 20 bp, Canis familiaris
tggcgggcggcatggcaggc

SEQ ID NO: 464 - DNA, 25 bp, Canis familiaris
ggcgggcggcatggcaggcgcgggc

SEQ ID NO: 465 - DNA, 24 bp, Canis familiaris
ggcgggcggcatggcaggcgcggg

SEQ ID NO: 466 - DNA, 23 bp, Canis familiaris
ggcgggcggcatggcaggcgcgg

SEQ ID NO: 467 - DNA, 22 bp, Canis familiaris
ggcgggcggcatggcaggcgcg

SEQ ID NO: 468 - DNA, 21 bp, Canis familiaris
ggcgggcggcatggcaggcgc

SEQ ID NO: 469 - DNA, 20 bp, Canis familiaris
ggcgggcggcatggcaggcg

SEQ ID NO: 470 - DNA, 25 bp, Canis familiaris
gcgggcggcatggcaggcgcgggcc

SEQ ID NO: 471 - DNA, 24 bp, Canis familiaris
gcgggcggcatggcaggcgcgggc

SEQ ID NO: 472 - DNA, 23 bp, Canis familiaris
gcgggcggcatggcaggcgcggg

SEQ ID NO: 473 - DNA, 22 bp, Canis familiaris
gcgggcggcatggcaggcgcgg

SEQ ID NO: 474 - DNA, 21 bp, Canis familiaris
gcgggcggcatggcaggcgcg

SEQ ID NO: 475 - DNA, 20 bp, Canis familiaris
gcgggcggcatggcaggcgc

SEQ ID NO: 476 - DNA, 25 bp, Canis familiaris
cgggcggcatggcaggcgcgggccg

SEQ ID NO: 477 - DNA, 24 bp, Canis familiaris
cgggcggcatggcaggcgcgggcc

SEQ ID NO: 478 - DNA, 23 bp, Canis familiaris
cgggcggcatggcaggcgcgggc

SEQ ID NO: 479 - DNA, 22 bp, Canis familiaris
cgggcggcatggcaggcgcggg

SEQ ID NO: 480 - DNA, 21 bp, Canis familiaris
cgggcggcatggcaggcgcgg

SEQ ID NO: 481 - DNA, 20 bp, Canis familiaris
cgggcggcatggcaggcgcg

SEQ ID NO: 482 - DNA, 25 bp, Canis familiaris
gggcggcatggcaggcgcgggccgg

SEQ ID NO: 483 - DNA, 24 bp, Canis familiaris
gggcggcatggcaggcgcgggccg

SEQ ID NO: 484 - DNA, 23 bp, Canis familiaris
gggcggcatggcaggcgcgggcc

SEQ ID NO: 485 - DNA, 22 bp, Canis familiaris
gggcggcatggcaggcgcgggc

SEQ ID NO: 486 - DNA, 21 bp, Canis familiaris
gggcggcatggcaggcgcggg

SEQ ID NO: 487 - DNA, 20 bp, Canis familiaris
gggcggcatggcaggcgcgg

SEQ ID NO: 488 - DNA, 25 bp, Canis familiaris
ggcggcatggcaggcgcgggccggg

SEQ ID NO: 489 - DNA, 24 bp, Canis familiaris
ggcggcatggcaggcgcgggccgg

SEQ ID NO: 490 - DNA, 23 bp, Canis familiaris
ggcggcatggcaggcgcgggccg

SEQ ID NO: 491 - DNA, 22 bp, Canis familiaris
ggcggcatggcaggcgcgggcc

SEQ ID NO: 492 - DNA, 21 bp, Canis familiaris
ggcggcatggcaggcgcgggc

SEQ ID NO: 493 - DNA, 20 bp, Canis familiaris
ggcggcatggcaggcgcggg

SEQ ID NO: 494 - DNA, 25 bp, Canis familiaris
gcggcatggcaggcgcgggccgggc

SEQ ID NO: 495 - DNA, 24 bp, Canis familiaris
gcggcatggcaggcgcgggccggg

SEQ ID NO: 496 - DNA, 23 bp, Canis familiaris
gcggcatggcaggcgcgggccgg

SEQ ID NO: 497 - DNA, 22 bp, Canis familiaris
gcggcatggcaggcgcgggccg

SEQ ID NO: 498 - DNA, 21 bp, Canis familiaris
gcggcatggcaggcgcgggcc

SEQ ID NO: 499 - DNA, 20 bp, Canis familiaris
gcggcatggcaggcgcgggc

SEQ ID NO: 500 - DNA, 25 bp, Canis familiaris
cggcctcggccatggcggcggcggc

SEQ ID NO: 501 - DNA, 24 bp, Canis familiaris
cggcctcggccatggcggcggcgg

SEQ ID NO: 502 - DNA, 23 bp, Canis familiaris
cggcctcggccatggcggcggcg

SEQ ID NO: 503 - DNA, 22 bp, Canis familiaris
cggcctcggccatggcggcggc

SEQ ID NO: 504 - DNA, 21 bp, Canis familiaris
cggcctcggccatggcggcgg

SEQ ID NO: 505 - DNA, 20 bp, Canis familiaris
cggcctcggccatggcggcg

SEQ ID NO: 506 - DNA, 25 bp, Canis familiaris
ggcctcggccatggcggcggcggcg

SEQ ID NO: 507 - DNA, 24 bp, Canis familiaris
ggcctcggccatggcggcggcggc

SEQ ID NO: 508 - DNA, 23 bp, Canis familiaris
ggcctcggccatggcggcggcgg

SEQ ID NO: 509 - DNA, 22 bp, Canis familiaris
ggcctcggccatggcggcggcg

SEQ ID NO: 510 - DNA, 21 bp, Canis familiaris
ggcctcggccatggcggcggc

SEQ ID NO: 511 - DNA, 20 bp, Canis familiaris
ggcctcggccatggcggcgg

SEQ ID NO: 512 - DNA, 25 bp, Canis familiaris
gcctcggccatggcggcggcggcgg

SEQ ID NO: 513 - DNA, 24 bp, Canis familiaris
gcctcggccatggcggcggcggcg

SEQ ID NO: 514 - DNA, 23 bp, Canis familiaris
gcctcggccatggcggcggcggc

SEQ ID NO: 515 - DNA, 22 bp, Canis familiaris
gcctcggccatggcggcggcgg

SEQ ID NO: 516 - DNA, 21 bp, Canis familiaris
gcctcggccatggcggcggcg

SEQ ID NO: 517 - DNA, 20 bp, Canis familiaris
gcctcggccatggcggcggc

SEQ ID NO: 518 - DNA, 25 bp, Canis familiaris
cctcggccatggcggcggcggcggc

SEQ ID NO: 519 - DNA, 24 bp, Canis familiaris
cctcggccatggcggcggcggcgg

SEQ ID NO: 520 - DNA, 23 bp, Canis familiaris
cctcggccatggcggcggcggcg

SEQ ID NO: 521 - DNA, 22 bp, Canis familiaris
cctcggccatggcggcggcggc

SEQ ID NO: 522 - DNA, 21 bp, Canis familiaris
cctcggccatggcggcggcgg

SEQ ID NO: 523 - DNA, 20 bp, Canis familiaris
cctcggccatggcggcggcg

SEQ ID NO: 524 - DNA, 25 bp, Canis familiaris
ctcggccatggcggcggcggcggcg

SEQ ID NO: 525 - DNA, 24 bp, Canis familiaris
ctcggccatggcggcggcggcggc

SEQ ID NO: 526 - DNA, 23 bp, Canis familiaris
ctcggccatggcggcggcggcgg

SEQ ID NO: 527 - DNA, 22 bp, Canis familiaris
ctcggccatggcggcggcggcg

SEQ ID NO: 528 - DNA, 21 bp, Canis familiaris
ctcggccatggcggcggcggc

SEQ ID NO: 529 - DNA, 20 bp, Canis familiaris
ctcggccatggcggcggcgg

SEQ ID NO: 530 - DNA, 25 bp, Canis familiaris
tcggccatggcggcggcggcggcgg

SEQ ID NO: 531 - DNA, 24 bp, Canis familiaris
tcggccatggcggcggcggcggcg

SEQ ID NO: 532 - DNA, 23 bp, Canis familiaris
tcggccatggcggcggcggcggc

SEQ ID NO: 533 - DNA, 22 bp, Canis familiaris
tcggccatggcggcggcggcgg

SEQ ID NO: 534 - DNA, 21 bp, Canis familiaris
tcggccatggcggcggcggcg

SEQ ID NO: 535 - DNA, 20 bp, Canis familiaris
tcggccatggcggcggcggc

SEQ ID NO: 536 - DNA, 25 bp, Canis familiaris
cggccatggcggcggcggcggcggc

SEQ ID NO: 537 - DNA, 24 bp, Canis familiaris
cggccatggcggcggcggcggcgg

SEQ ID NO: 538 - DNA, 23 bp, Canis familiaris
cggccatggcggcggcggcggcg

SEQ ID NO: 539 - DNA, 22 bp, Canis familiaris
cggccatggcggcggcggcggc

SEQ ID NO: 540 - DNA, 21 bp, Canis familiaris
cggccatggcggcggcggcgg

SEQ ID NO: 541 - DNA, 20 bp, Canis familiaris
cggccatggcggcggcggcg

SEQ ID NO: 542 - DNA, 26 bp, Canis familiaris
cggccatggcggcggcggcggcggcg

SEQ ID NO: 543 - DNA, 25 bp, Canis familiaris
ggccatggcggcggcggcggcggcg

SEQ ID NO: 544 - DNA, 24 bp, Canis familiaris
ggccatggcggcggcggcggcggc

SEQ ID NO: 545 - DNA, 23 bp, Canis familiaris
ggccatggcggcggcggcggcgg

SEQ ID NO: 546 - DNA, 22 bp, Canis familiaris
ggccatggcggcggcggcggcg

SEQ ID NO: 547 - DNA, 21 bp, Canis familiaris
ggccatggcggcggcggcggc

SEQ ID NO: 548 - DNA, 20 bp, Canis familiaris
ggccatggcggcggcggcgg

SEQ ID NO: 549 - DNA, 25 bp, Canis familiaris
gccatggcggcggcggcggcggcgg

SEQ ID NO: 550 - DNA, 24 bp, Canis familiaris
gccatggcggcggcggcggcggcg

SEQ ID NO: 551 - DNA, 23 bp, Canis familiaris
gccatggcggcggcggcggcggc

SEQ ID NO: 552 - DNA, 22 bp, Canis familiaris
gccatggcggcggcggcggcgg

SEQ ID NO: 553 - DNA, 21 bp, Canis familiaris
gccatggcggcggcggcggcg

SEQ ID NO: 554 - DNA, 20 bp, Canis familiaris
gccatggcggcggcggcggc

SEQ ID NO: 555 - DNA, 25 bp, Canis familiaris
ccatggcggcggcggcggcggcggc

SEQ ID NO: 556 - DNA, 24 bp, Canis familiaris
ccatggcggcggcggcggcggcgg

SEQ ID NO: 557 - DNA, 23 bp, Canis familiaris
ccatggcggcggcggcggcggcg

SEQ ID NO: 558 - DNA, 22 bp, Canis familiaris
ccatggcggcggcggcggcggc

SEQ ID NO: 559 - DNA, 21 bp, Canis familiaris
ccatggcggcggcggcggcgg

SEQ ID NO: 560 - DNA, 20 bp, Canis familiaris
ccatggcggcggcggcggcg

SEQ ID NO: 561 - DNA, 25 bp, Canis familiaris
cgcccagcgtgcgccatcctccccc

SEQ ID NO: 562 - DNA, 24 bp, Canis familiaris
cgcccagcgtgcgccatcctcccc

SEQ ID NO: 563 - DNA, 23 bp, Canis familiaris
cgcccagcgtgcgccatcctccc

SEQ ID NO: 564 - DNA, 22 bp, Canis familiaris
cgcccagcgtgcgccatcctcc

SEQ ID NO: 565 - DNA, 21 bp, Canis familiaris
cgcccagcgtgcgccatcctc

SEQ ID NO: 566 - DNA, 20 bp, Canis familiaris
cgcccagcgtgcgccatcct

SEQ ID NO: 567 - DNA, 25 bp, Canis familiaris
gcccagcgtgcgccatcctcccccg

SEQ ID NO: 568 - DNA, 24 bp, Canis familiaris
gcccagcgtgcgccatcctccccc

SEQ ID NO: 569 - DNA, 23 bp, Canis familiaris
gcccagcgtgcgccatcctcccc

SEQ ID NO: 570 - DNA, 22 bp, Canis familiaris
gcccagcgtgcgccatcctccc

SEQ ID NO: 571 - DNA, 21 bp, Canis familiaris
gcccagcgtgcgccatcctcc

SEQ ID NO: 572 - DNA, 20 bp, Canis familiaris
gcccagcgtgcgccatcctc

SEQ ID NO: 573 - DNA, 25 bp, Canis familiaris
cccagcgtgcgccatcctcccccgg

SEQ ID NO: 574 - DNA, 24 bp, Canis familiaris
cccagcgtgcgccatcctcccccg

SEQ ID NO: 575 - DNA, 23 bp, Canis familiaris
cccagcgtgcgccatcctccccc

SEQ ID NO: 576 - DNA, 22 bp, Canis familiaris
cccagcgtgcgccatcctcccc

SEQ ID NO: 577 - DNA, 21 bp, Canis familiaris
cccagcgtgcgccatcctccc

SEQ ID NO: 578 - DNA, 20 bp, Canis familiaris
cccagcgtgcgccatcctcc

SEQ ID NO: 579 - DNA, 25 bp, Canis familiaris
ccagcgtgcgccatcctcccccggg

SEQ ID NO: 580 - DNA, 24 bp, Canis familiaris
ccagcgtgcgccatcctcccccgg

SEQ ID NO: 581 - DNA, 23 bp, Canis familiaris
ccagcgtgcgccatcctcccccg

SEQ ID NO: 582 - DNA, 22 bp, Canis familiaris
ccagcgtgcgccatcctccccc

SEQ ID NO: 583 - DNA, 21 bp, Canis familiaris
ccagcgtgcgccatcctcccc

SEQ ID NO: 584 - DNA, 20 bp, Canis familiaris
ccagcgtgcgccatcctccc

SEQ ID NO: 585 - DNA, 24 bp, Canis familiaris
cagcgtgcgccatcctcccccggg

SEQ ID NO: 586 - DNA, 23 bp, Canis familiaris
cagcgtgcgccatcctcccccgg

SEQ ID NO: 587 - DNA, 22 bp, Canis familiaris
cagcgtgcgccatcctcccccg

SEQ ID NO: 588 - DNA, 21 bp, Canis familiaris
cagcgtgcgccatcctccccc

SEQ ID NO: 589 - DNA, 20 bp, Canis familiaris
cagcgtgcgccatcctcccc

SEQ ID NO: 590 - DNA, 23 bp, Canis familiaris
agcgtgcgccatcctcccccggg

SEQ ID NO: 591 - DNA, 22 bp, Canis familiaris
agcgtgcgccatcctcccccgg

SEQ ID NO: 592 - DNA, 21 bp, Canis familiaris
agcgtgcgccatcctcccccg

SEQ ID NO: 593 - DNA, 20 bp, Canis familiaris
agcgtgcgccatcctccccc

SEQ ID NO: 594 - DNA, 22 bp, Canis familiaris
gcgtgcgccatcctcccccggg

SEQ ID NO: 595 - DNA, 21 bp, Canis familiaris
gcgtgcgccatcctcccccgg

SEQ ID NO: 596 - DNA, 20 bp, Canis familiaris
gcgtgcgccatcctcccccg

SEQ ID NO: 597 - DNA, 21 bp, Canis familiaris
cgtgcgccatcctcccccggg

SEQ ID NO: 598 - DNA, 20 bp, Canis familiaris
cgtgcgccatcctcccccgg

SEQ ID NO: 599 - DNA, 20 bp, Canis familiaris
gtgcgccatcctcccccggg

SEQ ID NO: 600 - DNA, 25 bp, Canis familiaris
cctcttcatccatgtgtgttccagg

SEQ ID NO: 601 - DNA, 24 bp, Canis familiaris
cctcttcatccatgtgtgttccag

SEQ ID NO: 602 - DNA, 23 bp, Canis familiaris
cctcttcatccatgtgtgttcca

SEQ ID NO: 603 - DNA, 22 bp, Canis familiaris
cctcttcatccatgtgtgttcc

SEQ ID NO: 604 - DNA, 21 bp, Canis familiaris
cctcttcatccatgtgtgttc

SEQ ID NO: 605 - DNA, 20 bp, Canis familiaris
cctcttcatccatgtgtgtt

SEQ ID NO: 606 - DNA, 24 bp, Canis familiaris
ctcttcatccatgtgtgttccagg

SEQ ID NO: 607 - DNA, 23 bp, Canis familiaris
ctcttcatccatgtgtgttccag

SEQ ID NO: 608 - DNA, 22 bp, Canis familiaris
ctcttcatccatgtgtgttcca

SEQ ID NO: 609 - DNA, 21 bp, Canis familiaris
ctcttcatccatgtgtgttcc

SEQ ID NO: 610 - DNA, 20 bp, Canis familiaris
ctcttcatccatgtgtgttc

SEQ ID NO: 611 - DNA, 23 bp, Canis familiaris
tcttcatccatgtgtgttccagg

SEQ ID NO: 612 - DNA, 22 bp, Canis familiaris
tcttcatccatgtgtgttccag

SEQ ID NO: 613 - DNA, 21 bp, Canis familiaris
tcttcatccatgtgtgttcca

SEQ ID NO: 614 - DNA, 20 bp, Canis familiaris
tcttcatccatgtgtgttcc

SEQ ID NO: 615 - DNA, 22 bp, Canis familiaris
cttcatccatgtgtgttccagg

SEQ ID NO: 616 - DNA, 21 bp, Canis familiaris
cttcatccatgtgtgttccag

SEQ ID NO: 617 - DNA, 20 bp, Canis familiaris
cttcatccatgtgtgttcca

SEQ ID NO: 618 - DNA, 21 bp, Canis familiaris
ttcatccatgtgtgttccagg

SEQ ID NO: 619 - DNA, 20 bp, Canis familiaris
ttcatccatgtgtgttccag

SEQ ID NO: 620 - DNA, 20 bp, Canis familiaris
tcatccatgtgtgttccagg

SEQ ID NO: 621 - DNA, 25 bp, Canis familiaris
cccagtctggcccatgattattctg

SEQ ID NO: 622 - DNA, 23 bp, Canis familiaris
cccagtctggcccatgattattc

SEQ ID NO: 623 - DNA, 22 bp, Canis familiaris
cccagtctggcccatgattatt

SEQ ID NO: 624 - DNA, 21 bp, Canis familiaris
cccagtctggcccatgattat

SEQ ID NO: 625 - DNA, 20 bp, Canis familiaris
cccagtctggcccatgatta

SEQ ID NO: 626 - DNA, 25 bp, Canis familiaris
ccagtctggcccatgattattctgc

SEQ ID NO: 627 - DNA, 24 bp, Canis familiaris
ccagtctggcccatgattattctg

SEQ ID NO: 628 - DNA, 23 bp, Canis familiaris
ccagtctggcccatgattattct

SEQ ID NO: 629 - DNA, 22 bp, Canis familiaris
ccagtctggcccatgattattc

SEQ ID NO: 630 - DNA, 21 bp, Canis familiaris
ccagtctggcccatgattatt

SEQ ID NO: 631 - DNA, 20 bp, Canis familiaris
ccagtctggcccatgattat

SEQ ID NO: 632 - DNA, 25 bp, Canis familiaris
cagtctggcccatgattattctgcg

SEQ ID NO: 633 - DNA, 24 bp, Canis familiaris
cagtctggcccatgattattctgc

SEQ ID NO: 634 - DNA, 23 bp, Canis familiaris
cagtctggcccatgattattctg

SEQ ID NO: 635 - DNA, 22 bp, Canis familiaris
cagtctggcccatgattattct

SEQ ID NO: 636 - DNA, 21 bp, Canis familiaris
cagtctggcccatgattattc

SEQ ID NO: 637 - DNA, 20 bp, Canis familiaris
cagtctggcccatgattatt

SEQ ID NO: 638 - DNA, 25 bp, Canis familiaris
agtctggcccatgattattctgcgc

SEQ ID NO: 639 - DNA, 24 bp, Canis familiaris
agtctggcccatgattattctgcg

SEQ ID NO: 640 - DNA, 23 bp, Canis familiaris
agtctggcccatgattattctgc

SEQ ID NO: 641 - DNA, 22 bp, Canis familiaris
agtctggcccatgattattctg

SEQ ID NO: 642 - DNA, 21 bp, Canis familiaris
agtctggcccatgattattct

SEQ ID NO: 643 - DNA, 20 bp, Canis familiaris
agtctggcccatgattattc

SEQ ID NO: 644 - DNA, 25 bp, Canis familiaris
gtctggcccatgattattctgcgcc

SEQ ID NO: 645 - DNA, 24 bp, Canis familiaris
gtctggcccatgattattctgcgc

SEQ ID NO: 646 - DNA, 23 bp, Canis familiaris
gtctggcccatgattattctgcg

SEQ ID NO: 647 - DNA, 22 bp, Canis familiaris
gtctggcccatgattattctgc

SEQ ID NO: 648 - DNA, 21 bp, Canis familiaris
gtctggcccatgattattctg

SEQ ID NO: 649 - DNA, 20 bp, Canis familiaris
gtctggcccatgattattct

SEQ ID NO: 650 - DNA, 25 bp, Canis familiaris
tctggcccatgattattctgcgccc

SEQ ID NO: 651 - DNA, 24 bp, Canis familiaris
tctggcccatgattattctgcgcc

SEQ ID NO: 652 - DNA, 23 bp, Canis familiaris
tctggcccatgattattctgcgc

SEQ ID NO: 653 - DNA, 22 bp, Canis familiaris
tctggcccatgattattctgcg

SEQ ID NO: 654 - DNA, 21 bp, Canis familiaris
tctggcccatgattattctgc

SEQ ID NO: 655 - DNA, 20 bp, Canis familiaris
tctggcccatgattattctg

SEQ ID NO: 656 - DNA, 25 bp, Canis familiaris
ctggcccatgattattctgcgcccc

SEQ ID NO: 657 - DNA, 24 bp, Canis familiaris
ctggcccatgattattctgcgccc

SEQ ID NO: 658 - DNA, 23 bp, Canis familiaris
ctggcccatgattattctgcgcc

SEQ ID NO: 659 - DNA, 22 bp, Canis familiaris
ctggcccatgattattctgcgc

SEQ ID NO: 660 - DNA, 21 bp, Canis familiaris
ctggcccatgattattctgcg

SEQ ID NO: 661 - DNA, 20 bp, Canis familiaris
ctggcccatgattattctgc

SEQ ID NO: 662 - DNA, 25 bp, Canis familiaris
tggcccatgattattctgcgccccg

SEQ ID NO: 663 - DNA, 24 bp, Canis familiaris
tggcccatgattattctgcgcccc

SEQ ID NO: 664 - DNA, 23 bp, Canis familiaris
tggcccatgattattctgcgccc

SEQ ID NO: 665 - DNA, 22 bp, Canis familiaris
tggcccatgattattctgcgcc

SEQ ID NO: 666 - DNA, 21 bp, Canis familiaris
tggcccatgattattctgcgc

SEQ ID NO: 667 - DNA, 20 bp, Canis familiaris
tggcccatgattattctgcg

SEQ ID NO: 668 - DNA, 25 bp, Canis familiaris
ggcccatgattattctgcgccccgc

SEQ ID NO: 669 - DNA, 24 bp, Canis familiaris
ggcccatgattattctgcgccccg

SEQ ID NO: 670 - DNA, 23 bp, Canis familiaris
ggcccatgattattctgcgcccc

SEQ ID NO: 671 - DNA, 22 bp, Canis familiaris
ggcccatgattattctgcgccc

SEQ ID NO: 672 - DNA, 21 bp, Canis familiaris
ggcccatgattattctgcgcc

SEQ ID NO: 673 - DNA, 20 bp, Canis familiaris
ggcccatgattattctgcgc

SEQ ID NO: 674 - DNA, 25 bp, Canis familiaris
gcccatgattattctgcgccccgcg

SEQ ID NO: 675 - DNA, 24 bp, Canis familiaris
gcccatgattattctgcgccccgc

SEQ ID NO: 676 - DNA, 23 bp, Canis familiaris
gcccatgattattctgcgccccg

SEQ ID NO: 677 - DNA, 22 bp, Canis familiaris
gcccatgattattctgcgcccc

SEQ ID NO: 678 - DNA, 21 bp, Canis familiaris
gcccatgattattctgcgccc

SEQ ID NO: 679 - DNA, 20 bp, Canis familiaris
gcccatgattattctgcgcc

SEQ ID NO: 680 - DNA, 25 bp, Canis familiaris
cacgaactccatgctggcggcaggc

SEQ ID NO: 681 - DNA, 24 bp, Canis familiaris
cacgaactccatgctggcggcagg

SEQ ID NO: 682 - DNA, 23 bp, Canis familiaris
cacgaactccatgctggcggcag

SEQ ID NO: 683 - DNA, 22 bp, Canis familiaris
cacgaactccatgctggcggca

SEQ ID NO: 684 - DNA, 21 bp, Canis familiaris
cacgaactccatgctggcggc

SEQ ID NO: 685 - DNA, 20 bp, Canis familiaris
cacgaactccatgctggcgg

SEQ ID NO: 686 - DNA, 25 bp, Canis familiaris
acgaactccatgctggcggcaggcg

SEQ ID NO: 687 - DNA, 24 bp, Canis familiaris
acgaactccatgctggcggcaggc

SEQ ID NO: 688 - DNA, 23 bp, Canis familiaris
acgaactccatgctggcggcagg

SEQ ID NO: 689 - DNA, 22 bp, Canis familiaris
acgaactccatgctggcggcag

SEQ ID NO: 690 - DNA, 21 bp, Canis familiaris
acgaactccatgctggcggca

SEQ ID NO: 691 - DNA, 20 bp, Canis familiaris
acgaactccatgctggcggc

SEQ ID NO: 692 - DNA, 19 bp, Canis familiaris
acgaactccatgctggcgg

SEQ ID NO: 693 - DNA, 25 bp, Canis familiaris
cgaactccatgctggcggcaggcgg

SEQ ID NO: 694 - DNA, 24 bp, Canis familiaris
cgaactccatgctggcggcaggcg

SEQ ID NO: 695 - DNA, 23 bp, Canis familiaris
cgaactccatgctggcggcaggc

SEQ ID NO: 696 - DNA, 22 bp, Canis familiaris
cgaactccatgctggcggcagg

SEQ ID NO: 697 - DNA, 21 bp, Canis familiaris
cgaactccatgctggcggcag

SEQ ID NO: 698 - DNA, 20 bp, Canis familiaris
cgaactccatgctggcggca

SEQ ID NO: 699 - DNA, 25 bp, Canis familiaris
gaactccatgctggcggcaggcggc

SEQ ID NO: 700 - DNA, 24 bp, Canis familiaris
gaactccatgctggcggcaggcgg

SEQ ID NO: 701 - DNA, 23 bp, Canis familiaris
gaactccatgctggcggcaggcg

SEQ ID NO: 702 - DNA, 22 bp, Canis familiaris
gaactccatgctggcggcaggc

SEQ ID NO: 703 - DNA, 21 bp, Canis familiaris
gaactccatgctggcggcagg

SEQ ID NO: 704 - DNA, 25 bp, Canis familiaris
gggctgcgccccgccggctcattta

SEQ ID NO: 705 - DNA, 24 bp, Canis familiaris
gggctgcgccccgccggctcattt

SEQ ID NO: 706 - DNA, 23 bp, Canis familiaris
gggctgcgccccgccggctcatt

SEQ ID NO: 707 - DNA, 22 bp, Canis familiaris
gggctgcgccccgccggctcat

SEQ ID NO: 708 - DNA, 21 bp, Canis familiaris
gggctgcgccccgccggctca

SEQ ID NO: 709 - DNA, 20 bp, Canis familiaris
gggctgcgccccgccggctc

SEQ ID NO: 710 - DNA, 25 bp, Canis familiaris
ggctgcgccccgccggctcatttaa

SEQ ID NO: 711 - DNA, 24 bp, Canis familiaris
ggctgcgccccgccggctcattta

SEQ ID NO: 712 - DNA, 23 bp, Canis familiaris
ggctgcgccccgccggctcattt

SEQ ID NO: 713 - DNA, 22 bp, Canis familiaris
ggctgcgccccgccggctcatt

SEQ ID NO: 714 - DNA, 21 bp, Canis familiaris
ggctgcgccccgccggctcat

SEQ ID NO: 715 - DNA, 20 bp, Canis familiaris
ggctgcgccccgccggctca

SEQ ID NO: 716 - DNA, 25 bp, Canis familiaris
cccgccggctcatttaaccagcggg

SEQ ID NO: 717 - DNA, 24 bp, Canis familiaris
cccgccggctcatttaaccagcgg

SEQ ID NO: 718 - DNA, 23 bp, Canis familiaris
cccgccggctcatttaaccagcg

SEQ ID NO: 719 - DNA, 22 bp, Canis familiaris
cccgccggctcatttaaccagc

SEQ ID NO: 720 - DNA, 21 bp, Canis familiaris
cccgccggctcatttaaccag

SEQ ID NO: 721 - DNA, 20 bp, Canis familiaris
cccgccggctcatttaacca

SEQ ID NO: 722 - DNA, 24 bp, Canis familiaris
ccgccggctcatttaaccagcggg

SEQ ID NO: 723 - DNA, 23 bp, Canis familiaris
ccgccggctcatttaaccagcgg

SEQ ID NO: 724 - DNA, 22 bp, Canis familiaris
ccgccggctcatttaaccagcg

SEQ ID NO: 725 - DNA, 21 bp, Canis familiaris
ccgccggctcatttaaccagc

SEQ ID NO: 726 - DNA, 20 bp, Canis familiaris
ccgccggctcatttaaccag

SEQ ID NO: 727 - DNA, 23 bp, Canis familiaris
cgccggctcatttaaccagcggg

SEQ ID NO: 728 - DNA, 22 bp, Canis familiaris
cgccggctcatttaaccagcgg

SEQ ID NO: 729 - DNA, 21 bp, Canis familiaris
cgccggctcatttaaccagcg

SEQ ID NO: 730 - DNA, 20 bp, Canis familiaris
cgccggctcatttaaccagc

SEQ ID NO: 731 - DNA, 22 bp, Canis familiaris
gccggctcatttaaccagcggg

SEQ ID NO: 732 - DNA, 21 bp, Canis familiaris
gccggctcatttaaccagcgg

SEQ ID NO: 733 - DNA, 20 bp, Canis familiaris
gccggctcatttaaccagcg

SEQ ID NO: 734 - DNA, 21 bp, Canis familiaris
ccggctcatttaaccagcggg

SEQ ID NO: 735 - DNA, 20 bp, Canis familiaris
ccggctcatttaaccagcgg

SEQ ID NO: 736 - DNA, 20 bp, Canis familiaris
cggctcatttaaccagcggg

SEQ ID NO: 737 - DNA, 25 bp, Canis familiaris
cgcttgatcacgtgcatcgcggcta

SEQ ID NO: 738 - DNA, 24 bp, Canis familiaris
cgcttgatcacgtgcatcgcggct

SEQ ID NO: 739 - DNA, 23 bp, Canis familiaris
cgcttgatcacgtgcatcgcggc

SEQ ID NO: 740 - DNA, 22 bp, Canis familiaris
cgcttgatcacgtgcatcgcgg

SEQ ID NO: 741 - DNA, 21 bp, Canis familiaris
cgcttgatcacgtgcatcgcg

SEQ ID NO: 742 - DNA, 20 bp, Canis familiaris
cgcttgatcacgtgcatcgc

SEQ ID NO: 743 - DNA, 25 bp, Canis familiaris
gcttgatcacgtgcatcgcggctag

SEQ ID NO: 744 - DNA, 24 bp, Canis familiaris
gcttgatcacgtgcatcgcggcta

SEQ ID NO: 745 - DNA, 23 bp, Canis familiaris
gcttgatcacgtgcatcgcggct

SEQ ID NO: 746 - DNA, 22 bp, Canis familiaris
gcttgatcacgtgcatcgcggc

SEQ ID NO: 747 - DNA, 21 bp, Canis familiaris
gcttgatcacgtgcatcgcgg

SEQ ID NO: 748 - DNA, 20 bp, Canis familiaris
gcttgatcacgtgcatcgcg

SEQ ID NO: 749 - DNA, 25 bp, Canis familiaris
cttgatcacgtgcatcgcggctagt

SEQ ID NO: 750 - DNA, 24 bp, Canis familiaris
cttgatcacgtgcatcgcggctag

SEQ ID NO: 751 - DNA, 23 bp, Canis familiaris
cttgatcacgtgcatcgcggcta

SEQ ID NO: 752 - DNA, 22 bp, Canis familiaris
cttgatcacgtgcatcgcggct

SEQ ID NO: 753 - DNA, 21 bp, Canis familiaris
cttgatcacgtgcatcgcggc

SEQ ID NO: 754 - DNA, 20 bp, Canis familiaris
cttgatcacgtgcatcgcgg

SEQ ID NO: 755 - DNA, 25 bp, Canis familiaris
ttgatcacgtgcatcgcggctagtg

SEQ ID NO: 756 - DNA, 24 bp, Canis familiaris
ttgatcacgtgcatcgcggctagt

SEQ ID NO: 757 - DNA, 23 bp, Canis familiaris
ttgatcacgtgcatcgcggctag

SEQ ID NO: 758 - DNA, 22 bp, Canis familiaris
ttgatcacgtgcatcgcggcta

SEQ ID NO: 759 - DNA, 21 bp, Canis familiaris
ttgatcacgtgcatcgcggct

SEQ ID NO: 760 - DNA, 20 bp, Canis familiaris
ttgatcacgtgcatcgcggc

SEQ ID NO: 761 - DNA, 25 bp, Canis familiaris
tgatcacgtgcatcgcggctagtgg

SEQ ID NO: 762 - DNA, 24 bp, Canis familiaris
tgatcacgtgcatcgcggctagtg

SEQ ID NO: 763 - DNA, 23 bp, Canis familiaris
tgatcacgtgcatcgcggctagt

SEQ ID NO: 764 - DNA, 22 bp, Canis familiaris
tgatcacgtgcatcgcggctag

SEQ ID NO: 765 - DNA, 21 bp, Canis familiaris
tgatcacgtgcatcgcggcta

SEQ ID NO: 766 - DNA, 20 bp, Canis familiaris
tgatcacgtgcatcgcggct

SEQ ID NO: 767 - DNA, 25 bp, Canis familiaris
gatcacgtgcatcgcggctagtggc

SEQ ID NO: 768 - DNA, 24 bp, Canis familiaris
gatcacgtgcatcgcggctagtgg

SEQ ID NO: 769 - DNA, 23 bp, Canis familiaris
gatcacgtgcatcgcggctagtg

SEQ ID NO: 770 - DNA, 22 bp, Canis familiaris
gatcacgtgcatcgcggctagt

SEQ ID NO: 771 - DNA, 21 bp, Canis familiaris
gatcacgtgcatcgcggctag

SEQ ID NO: 772 - DNA, 20 bp, Canis familiaris
gatcacgtgcatcgcggcta

SEQ ID NO: 773 - DNA, 24 bp, Canis familiaris
atcacgtgcatcgcggctagtggc

SEQ ID NO: 774 - DNA, 23 bp, Canis familiaris
atcacgtgcatcgcggctagtgg

SEQ ID NO: 775 - DNA, 22 bp, Canis familiaris
atcacgtgcatcgcggctagtg

SEQ ID NO: 776 - DNA, 21 bp, Canis familiaris
atcacgtgcatcgcggctagt

SEQ ID NO: 777 - DNA, 20 bp, Canis familiaris
atcacgtgcatcgcggctag

SEQ ID NO: 778 - DNA, 23 bp, Canis familiaris
tcacgtgcatcgcggctagtggc

SEQ ID NO: 779 - DNA, 22 bp, Canis familiaris
tcacgtgcatcgcggctagtgg

SEQ ID NO: 780 - DNA, 21 bp, Canis familiaris
tcacgtgcatcgcggctagtg

SEQ ID NO: 781 - DNA, 20 bp, Canis familiaris
tcacgtgcatcgcggctagt

SEQ ID NO: 782 - DNA, 22 bp, Canis familiaris
cacgtgcatcgcggctagtggc

SEQ ID NO: 783 - DNA, 21 bp, Canis familiaris
cacgtgcatcgcggctagtgg

SEQ ID NO: 784 - DNA, 20 bp, Canis familiaris
cacgtgcatcgcggctagtg

SEQ ID NO: 785 - DNA, 21 bp, Canis familiaris
acgtgcatcgcggctagtggc

SEQ ID NO: 786 - DNA, 20 bp, Canis familiaris
acgtgcatcgcggctagtgg

SEQ ID NO: 787 - DNA, 20 bp, Canis familiaris
cgtgcatcgcggctagtggc

SEQ ID NO: 788 - DNA, 25 bp, Canis familiaris
cgcggacggagagcatggcggcggc

SEQ ID NO: 789 - DNA, 24 bp, Canis familiaris
cgcggacggagagcatggcggcgg

SEQ ID NO: 790 - DNA, 23 bp, Canis familiaris
cgcggacggagagcatggcggcg

SEQ ID NO: 791 - DNA, 22 bp, Canis familiaris
cgcggacggagagcatggcggc

SEQ ID NO: 792 - DNA, 21 bp, Canis familiaris
cgcggacggagagcatggcgg

SEQ ID NO: 793 - DNA, 20 bp, Canis familiaris
cgcggacggagagcatggcg

SEQ ID NO: 794 - DNA, 25 bp, Canis familiaris
gcggacggagagcatggcggcggca

SEQ ID NO: 795 - DNA, 24 bp, Canis familiaris
gcggacggagagcatggcggcggc

SEQ ID NO: 796 - DNA, 23 bp, Canis familiaris
gcggacggagagcatggcggcgg

SEQ ID NO: 797 - DNA, 22 bp, Canis familiaris
gcggacggagagcatggcggcg

SEQ ID NO: 798 - DNA, 21 bp, Canis familiaris
gcggacggagagcatggcggc

SEQ ID NO: 799 - DNA, 20 bp, Canis familiaris
gcggacggagagcatggcgg

SEQ ID NO: 800 - DNA, 25 bp, Canis familiaris
cggacggagagcatggcggcggcag

SEQ ID NO: 801 - DNA, 24 bp, Canis familiaris
cggacggagagcatggcggcggca

SEQ ID NO: 802 - DNA, 23 bp, Canis familiaris
cggacggagagcatggcggcggc

SEQ ID NO: 803 - DNA, 22 bp, Canis familiaris
cggacggagagcatggcggcgg

SEQ ID NO: 804 - DNA, 21 bp, Canis familiaris
cggacggagagcatggcggcg

SEQ ID NO: 805 - DNA, 20 bp, Canis familiaris
cggacggagagcatggcggc

SEQ ID NO: 806 - DNA, 25 bp, Canis familiaris
ggacggagagcatggcggcggcagg

SEQ ID NO: 807 - DNA, 24 bp, Canis familiaris
ggacggagagcatggcggcggcag

SEQ ID NO: 808 - DNA, 23 bp, Canis familiaris
ggacggagagcatggcggcggca

SEQ ID NO: 809 - DNA, 22 bp, Canis familiaris
ggacggagagcatggcggcggc

SEQ ID NO: 810 - DNA, 21 bp, Canis familiaris
ggacggagagcatggcggcgg

SEQ ID NO: 811 - DNA, 20 bp, Canis familiaris
ggacggagagcatggcggcg

SEQ ID NO: 812 - DNA, 25 bp, Canis familiaris
gacggagagcatggcggcggcaggc

SEQ ID NO: 813 - DNA, 24 bp, Canis familiaris
gacggagagcatggcggcggcagg

SEQ ID NO: 814 - DNA, 23 bp, Canis familiaris
gacggagagcatggcggcggcag

SEQ ID NO: 815 - DNA, 22 bp, Canis familiaris
gacggagagcatggcggcggca

SEQ ID NO: 816 - DNA, 21 bp, Canis familiaris
gacggagagcatggcggcggc

SEQ ID NO: 817 - DNA, 20 bp, Canis familiaris
gacggagagcatggcggcgg

SEQ ID NO: 818 - DNA, 25 bp, Canis familiaris
acggagagcatggcggcggcaggcg

SEQ ID NO: 819 - DNA, 24 bp, Canis familiaris
acggagagcatggcggcggcaggc

SEQ ID NO: 820 - DNA, 23 bp, Canis familiaris
acggagagcatggcggcggcagg

SEQ ID NO: 821 - DNA, 22 bp, Canis familiaris
acggagagcatggcggcggcag

SEQ ID NO: 822 - DNA, 21 bp, Canis familiaris
acggagagcatggcggcggca

SEQ ID NO: 823 - DNA, 20 bp, Canis familiaris
acggagagcatggcggcggc

SEQ ID NO: 824 - DNA, 25 bp, Canis familiaris
cggagagcatggcggcggcaggcgg

SEQ ID NO: 825 - DNA, 24 bp, Canis familiaris
cggagagcatggcggcggcaggcg

SEQ ID NO: 826 - DNA, 23 bp, Canis familiaris
cggagagcatggcggcggcaggc

SEQ ID NO: 827 - DNA, 22 bp, Canis familiaris
cggagagcatggcggcggcagg

SEQ ID NO: 828 - DNA, 21 bp, Canis familiaris
cggagagcatggcggcggcag

SEQ ID NO: 829 - DNA, 20 bp, Canis familiaris
cggagagcatggcggcggca

SEQ ID NO: 830 - DNA, 25 bp, Canis familiaris
ggagagcatggcggcggcaggcggg

SEQ ID NO: 831 - DNA, 24 bp, Canis familiaris
ggagagcatggcggcggcaggcgg

SEQ ID NO: 832 - DNA, 23 bp, Canis familiaris
ggagagcatggcggcggcaggcg

SEQ ID NO: 833 - DNA, 22 bp, Canis familiaris
ggagagcatggcggcggcaggc

SEQ ID NO: 834 - DNA, 21 bp, Canis familiaris
ggagagcatggcggcggcagg

SEQ ID NO: 835 - DNA, 20 bp, Canis familiaris
ggagagcatggcggcggcag

SEQ ID NO: 836 - DNA, 24 bp, Canis familiaris
gagagcatggcggcggcaggcggg

SEQ ID NO: 837 - DNA, 23 bp, Canis familiaris
gagagcatggcggcggcaggcgg

SEQ ID NO: 838 - DNA, 22 bp, Canis familiaris
gagagcatggcggcggcaggcg

SEQ ID NO: 839 - DNA, 21 bp, Canis familiaris
gagagcatggcggcggcaggc

SEQ ID NO: 840 - DNA, 20 bp, Canis familiaris
gagagcatggcggcggcagg

SEQ ID NO: 841 - DNA, 25 bp, Canis familiaris
agagcatggcggcggcaggcgggct

SEQ ID NO: 842 - DNA, 24 bp, Canis familiaris
agagcatggcggcggcaggcgggc

SEQ ID NO: 843 - DNA, 23 bp, Canis familiaris
agagcatggcggcggcaggcggg

SEQ ID NO: 844 - DNA, 22 bp, Canis familiaris
agagcatggcggcggcaggcgg

SEQ ID NO: 845 - DNA, 21 bp, Canis familiaris
agagcatggcggcggcaggcg

SEQ ID NO: 846 - DNA, 20 bp, Canis familiaris
agagcatggcggcggcaggc

SEQ ID NO: 847 - DNA, 25 bp, Canis familiaris
gagcatggcggcggcaggcgggctc

SEQ ID NO: 848 - DNA, 24 bp, Canis familiaris
gagcatggcggcggcaggcgggct

SEQ ID NO: 849 - DNA, 23 bp, Canis familiaris
gagcatggcggcggcaggcgggc

SEQ ID NO: 850 - DNA, 22 bp, Canis familiaris
gagcatggcggcggcaggcggg

SEQ ID NO: 851 - DNA, 21 bp, Canis familiaris
gagcatggcggcggcaggcgg

SEQ ID NO: 852 - DNA, 20 bp, Canis familiaris
gagcatggcggcggcaggcg

SEQ ID NO: 853 - DNA, 25 bp, Canis familiaris
agcatggcggcggcaggcgggctcg

SEQ ID NO: 854 - DNA, 24 bp, Canis familiaris
agcatggcggcggcaggcgggctc

SEQ ID NO: 855 - DNA, 23 bp, Canis familiaris
agcatggcggcggcaggcgggct

SEQ ID NO: 856 - DNA, 22 bp, Canis familiaris
agcatggcggcggcaggcgggc

SEQ ID NO: 857 - DNA, 21 bp, Canis familiaris
agcatggcggcggcaggcggg

SEQ ID NO: 858 - DNA, 20 bp, Canis familiaris
agcatggcggcggcaggcgg

SEQ ID NO: 859 - DNA, 25 bp, Canis familiaris
gcatggcggcggcaggcgggctcgg

SEQ ID NO: 860 - DNA, 24 bp, Canis familiaris
gcatggcggcggcaggcgggctcg

SEQ ID NO: 861 - DNA, 23 bp, Canis familiaris
gcatggcggcggcaggcgggctc

SEQ ID NO: 862 - DNA, 22 bp, Canis familiaris
gcatggcggcggcaggcgggct

SEQ ID NO: 863 - DNA, 21 bp, Canis familiaris
gcatggcggcggcaggcgggc

SEQ ID NO: 864 - DNA, 20 bp, Canis familiaris
gcatggcggcggcaggcggg

SEQ ID NO: 865 - DNA, 25 bp, Canis familiaris
catggcggcggcaggcgggctcggc

SEQ ID NO: 866 - DNA, 24 bp, Canis familiaris
catggcggcggcaggcgggctcgg

SEQ ID NO: 867 - DNA, 23 bp, Canis familiaris
catggcggcggcaggcgggctcg

SEQ ID NO: 868 - DNA, 22 bp, Canis familiaris
catggcggcggcaggcgggctc

SEQ ID NO: 869 - DNA, 21 bp, Canis familiaris
catggcggcggcaggcgggct

SEQ ID NO: 870 - DNA, 20 bp, Canis familiaris
catggcggcggcaggcgggc

SEQ ID NO: 871 - DNA, 25 bp, Canis familiaris
atggcggcggcaggcgggctcggcg

SEQ ID NO: 872 - DNA, 24 bp, Canis familiaris
atggcggcggcaggcgggctcggc

SEQ ID NO: 873 - DNA, 23 bp, Canis familiaris
atggcggcggcaggcgggctcgg

SEQ ID NO: 874 - DNA, 22 bp, Canis familiaris
atggcggcggcaggcgggctcg

SEQ ID NO: 875 - DNA, 21 bp, Canis familiaris
atggcggcggcaggcgggctc

SEQ ID NO: 876 - DNA, 20 bp, Canis familiaris
atggcggcggcaggcgggct

SEQ ID NO: 877 - DNA, 25 bp, Canis familiaris
tggcggcggcaggcgggctcggcgg

SEQ ID NO: 878 - DNA, 24 bp, Canis familiaris
tggcggcggcaggcgggctcggcg

SEQ ID NO: 879 - DNA, 23 bp, Canis familiaris
tggcggcggcaggcgggctcggc

SEQ ID NO: 880 - DNA, 22 bp, Canis familiaris
tggcggcggcaggcgggctcgg

SEQ ID NO: 881 - DNA, 21 bp, Canis familiaris
tggcggcggcaggcgggctcg

SEQ ID NO: 882 - DNA, 20 bp, Canis familiaris
tggcggcggcaggcgggctc

SEQ ID NO: 883 - DNA, 25 bp, Canis familiaris
ggcggcggcaggcgggctcggcggc

SEQ ID NO: 884 - DNA, 24 bp, Canis familiaris
ggcggcggcaggcgggctcggcgg

SEQ ID NO: 885 - DNA, 23 bp, Canis familiaris
ggcggcggcaggcgggctcggcg

SEQ ID NO: 886 - DNA, 22 bp, Canis familiaris
ggcggcggcaggcgggctcggc

SEQ ID NO: 887 - DNA, 21 bp, Canis familiaris
ggcggcggcaggcgggctcgg

SEQ ID NO: 888 - DNA, 20 bp, Canis familiaris
ggcggcggcaggcgggctcg

SEQ ID NO: 889 - DNA, 25 bp, Canis familiaris
cctgtttatacattttccaaatatc

SEQ ID NO: 890 - DNA, 24 bp, Canis familiaris
cctgtttatacattttccaaatat

SEQ ID NO: 891 - DNA, 23 bp, Canis familiaris
cctgtttatacattttccaaata

SEQ ID NO: 892 - DNA, 22 bp, Canis familiaris
cctgtttatacattttccaaat

SEQ ID NO: 893 - DNA, 21 bp, Canis familiaris
cctgtttatacattttccaaa

SEQ ID NO: 894 - DNA, 20 bp, Canis familiaris
cctgtttatacattttccaa

SEQ ID NO: 895 - DNA, 25 bp, Canis familiaris
ctgtttatacattttccaaatatca

SEQ ID NO: 896 - DNA, 24 bp, Canis familiaris
ctgtttatacattttccaaatatc

SEQ ID NO: 897 - DNA, 23 bp, Canis familiaris
ctgtttatacattttccaaatat

SEQ ID NO: 898 - DNA, 22 bp, Canis familiaris
ctgtttatacattttccaaata

SEQ ID NO: 899 - DNA, 21 bp, Canis familiaris
ctgtttatacattttccaaat

SEQ ID NO: 900 - DNA, 20 bp, Canis familiaris
ctgtttatacattttccaaa

SEQ ID NO: 901 - DNA, 25 bp, Canis familiaris
tgtttatacattttccaaatatcag

SEQ ID NO: 902 - DNA, 24 bp, Canis familiaris
tgtttatacattttccaaatatca

SEQ ID NO: 903 - DNA, 23 bp, Canis familiaris
tgtttatacattttccaaatatc

SEQ ID NO: 904 - DNA, 22 bp, Canis familiaris
tgtttatacattttccaaatat

SEQ ID NO: 905 - DNA, 21 bp, Canis familiaris
tgtttatacattttccaaata

SEQ ID NO: 906 - DNA, 20 bp, Canis familiaris
tgtttatacattttccaaat

SEQ ID NO: 907 - DNA, 25 bp, Canis familiaris
gtttatacattttccaaatatcagg

SEQ ID NO: 908 - DNA, 24 bp, Canis familiaris
gtttatacattttccaaatatcag

SEQ ID NO: 909 - DNA, 23 bp, Canis familiaris
gtttatacattttccaaatatca

SEQ ID NO: 910 - DNA, 22 bp, Canis familiaris
gtttatacattttccaaatatc

SEQ ID NO: 911 - DNA, 21 bp, Canis familiaris
gtttatacattttccaaatat

SEQ ID NO: 912 - DNA, 20 bp, Canis familiaris
gtttatacattttccaaata

SEQ ID NO: 913 - DNA, 25 bp, Canis familiaris
tttatacattttccaaatatcaggg

SEQ ID NO: 914 - DNA, 24 bp, Canis familiaris
tttatacattttccaaatatcagg

SEQ ID NO: 915 - DNA, 23 bp, Canis familiaris
tttatacattttccaaatatcag

SEQ ID NO: 916 - DNA, 22 bp, Canis familiaris
tttatacattttccaaatatca

SEQ ID NO: 917 - DNA, 21 bp, Canis familiaris
tttatacattttccaaatatc

SEQ ID NO: 918 - DNA, 25 bp, Canis familiaris
gggcagtttgtccgacatgctggca

SEQ ID NO: 919 - DNA, 24 bp, Canis familiaris
gggcagtttgtccgacatgctggc

SEQ ID NO: 920 - DNA, 23 bp, Canis familiaris
gggcagtttgtccgacatgctgg

SEQ ID NO: 921 - DNA, 22 bp, Canis familiaris
gggcagtttgtccgacatgctg

SEQ ID NO: 922 - DNA, 21 bp, Canis familiaris
gggcagtttgtccgacatgct

SEQ ID NO: 923 - DNA, 20 bp, Canis familiaris
gggcagtttgtccgacatgc

SEQ ID NO: 924 - DNA, 25 bp, Canis familiaris
ggcagtttgtccgacatgctggcag

SEQ ID NO: 925 - DNA, 24 bp, Canis familiaris
ggcagtttgtccgacatgctggca

SEQ ID NO: 926 - DNA, 23 bp, Canis familiaris
ggcagtttgtccgacatgctggc

SEQ ID NO: 927 - DNA, 22 bp, Canis familiaris
ggcagtttgtccgacatgctgg

SEQ ID NO: 928 - DNA, 21 bp, Canis familiaris
ggcagtttgtccgacatgctg

SEQ ID NO: 929 - DNA, 20 bp, Canis familiaris
ggcagtttgtccgacatgct

SEQ ID NO: 930 - DNA, 25 bp, Canis familiaris
gcagtttgtccgacatgctggcagc

SEQ ID NO: 931 - DNA, 24 bp, Canis familiaris
gcagtttgtccgacatgctggcag

SEQ ID NO: 932 - DNA, 23 bp, Canis familiaris
gcagtttgtccgacatgctggca

SEQ ID NO: 933 - DNA, 22 bp, Canis familiaris
gcagtttgtccgacatgctggc

SEQ ID NO: 934 - DNA, 21 bp, Canis familiaris
gcagtttgtccgacatgctgg

SEQ ID NO: 935 - DNA, 20 bp, Canis familiaris
gcagtttgtccgacatgctg

SEQ ID NO: 936 - DNA, 25 bp, Canis familiaris
cagtttgtccgacatgctggcagcg

SEQ ID NO: 937 - DNA, 24 bp, Canis familiaris
cagtttgtccgacatgctggcagc

SEQ ID NO: 938 - DNA, 23 bp, Canis familiaris
cagtttgtccgacatgctggcag

SEQ ID NO: 939 - DNA, 22 bp, Canis familiaris
cagtttgtccgacatgctggca

SEQ ID NO: 940 - DNA, 21 bp, Canis familiaris
cagtttgtccgacatgctggc

SEQ ID NO: 941 - DNA, 20 bp, Canis familiaris
cagtttgtccgacatgctgg

SEQ ID NO: 942 - DNA, 25 bp, Canis familiaris
agtttgtccgacatgctggcagcgc

SEQ ID NO: 943 - DNA, 24 bp, Canis familiaris
agtttgtccgacatgctggcagcg

SEQ ID NO: 944 - DNA, 23 bp, Canis familiaris
agtttgtccgacatgctggcagc

SEQ ID NO: 945 - DNA, 22 bp, Canis familiaris
agtttgtccgacatgctggcag

SEQ ID NO: 946 - DNA, 21 bp, Canis familiaris
agtttgtccgacatgctggca

SEQ ID NO: 947 - DNA, 20 bp, Canis familiaris
agtttgtccgacatgctggc

SEQ ID NO: 948 - DNA, 25 bp, Canis familiaris
gtttgtccgacatgctggcagcgct

SEQ ID NO: 949 - DNA, 24 bp, Canis familiaris
gtttgtccgacatgctggcagcgc

SEQ ID NO: 950 - DNA, 23 bp, Canis familiaris
gtttgtccgacatgctggcagcg

SEQ ID NO: 951 - DNA, 22 bp, Canis familiaris
gtttgtccgacatgctggcagc

SEQ ID NO: 952 - DNA, 21 bp, Canis familiaris
gtttgtccgacatgctggcag

SEQ ID NO: 953 - DNA, 20 bp, Canis familiaris
gtttgtccgacatgctggca

SEQ ID NO: 954 - DNA, 25 bp, Canis familiaris
tttgtccgacatgctggcagcgctt

SEQ ID NO: 955 - DNA, 24 bp, Canis familiaris
tttgtccgacatgctggcagcgct

SEQ ID NO: 956 - DNA, 23 bp, Canis familiaris
tttgtccgacatgctggcagcgc

SEQ ID NO: 957 - DNA, 22 bp, Canis familiaris
tttgtccgacatgctggcagcg

SEQ ID NO: 958 - DNA, 21 bp, Canis familiaris
tttgtccgacatgctggcagc

SEQ ID NO: 959 - DNA, 20 bp, Canis familiaris
tttgtccgacatgctggcag

SEQ ID NO: 960 - DNA, 25 bp, Canis familiaris
ttgtccgacatgctggcagcgcttc

SEQ ID NO: 961 - DNA, 24 bp, Canis familiaris
ttgtccgacatgctggcagcgctt

SEQ ID NO: 962 - DNA, 23 bp, Canis familiaris
ttgtccgacatgctggcagcgct

SEQ ID NO: 963 - DNA, 22 bp, Canis familiaris
ttgtccgacatgctggcagcgc

SEQ ID NO: 964 - DNA, 22 bp, Canis familiaris
ttgtccgacatgctggcagcgc

SEQ ID NO: 965 - DNA, 20 bp, Canis familiaris
ttgtccgacatgctggcagc

SEQ ID NO: 966 - DNA, 25 bp, Canis familiaris
tgtccgacatgctggcagcgcttcc

SEQ ID NO: 967 - DNA, 24 bp, Canis familiaris
tgtccgacatgctggcagcgcttc

SEQ ID NO: 968 - DNA, 23 bp, Canis familiaris
tgtccgacatgctggcagcgctt

SEQ ID NO: 969 - DNA, 22 bp, Canis familiaris
tgtccgacatgctggcagcgct

SEQ ID NO: 970 - DNA, 21 bp, Canis familiaris
tgtccgacatgctggcagcgc

SEQ ID NO: 971 - DNA, 20 bp, Canis familiaris
tgtccgacatgctggcagcg

SEQ ID NO: 972 - DNA, 25 bp, Canis familiaris
gtccgacatgctggcagcgcttccg

SEQ ID NO: 973 - DNA, 24 bp, Canis familiaris
gtccgacatgctggcagcgcttcc

SEQ ID NO: 974 - DNA, 23 bp, Canis familiaris
gtccgacatgctggcagcgcttc

SEQ ID NO: 975 - DNA, 22 bp, Canis familiaris
gtccgacatgctggcagcgctt

SEQ ID NO: 976 - DNA, 21 bp, Canis familiaris
gtccgacatgctggcagcgct

SEQ ID NO: 977 - DNA, 20 bp, Canis familiaris
gtccgacatgctggcagcgc

SEQ ID NO: 978 - DNA, 25 bp, Canis familiaris
tccgacatgctggcagcgcttccgc

SEQ ID NO: 979 - DNA, 24 bp, Canis familiaris
tccgacatgctggcagcgcttccg

SEQ ID NO: 980 - DNA, 23 bp, Canis familiaris
tccgacatgctggcagcgcttcc

SEQ ID NO: 981 - DNA, 22 bp, Canis familiaris
tccgacatgctggcagcgcttc

SEQ ID NO: 982 - DNA, 21 bp, Canis familiaris
tccgacatgctggcagcgctt

SEQ ID NO: 983 - DNA, 20 bp, Canis familiaris
tccgacatgctggcagcgct

SEQ ID NO: 984 - DNA, 25 bp, Canis familiaris
ccctgtgggtcaggagcattgcagg

SEQ ID NO: 985 - DNA, 24 bp, Canis familiaris
ccctgtgggtcaggagcattgcag

SEQ ID NO: 986 - DNA, 23 bp, Canis familiaris
ccctgtgggtcaggagcattgca

SEQ ID NO: 987 - DNA, 22 bp, Canis familiaris
ccctgtgggtcaggagcattgc

SEQ ID NO: 988 - DNA, 21 bp, Canis familiaris
ccctgtgggtcaggagcattg

SEQ ID NO: 989 - DNA, 20 bp, Canis familiaris
ccctgtgggtcaggagcatt

SEQ ID NO: 990 - DNA, 24 bp, Canis familiaris
gggtcaggagcattgcagggcccc

SEQ ID NO: 991 - DNA, 23 bp, Canis familiaris
gggtcaggagcattgcagggccc

SEQ ID NO: 992 - DNA, 22 bp, Canis familiaris
gggtcaggagcattgcagggcc

SEQ ID NO: 993 - DNA, 21 bp, Canis familiaris
gggtcaggagcattgcagggc

SEQ ID NO: 994 - DNA, 20 bp, Canis familiaris
gggtcaggagcattgcaggg

SEQ ID NO: 995 - DNA, 26 bp, Canis familiaris
gggtggccatgccgggcttcgcggcg

SEQ ID NO: 996 - DNA, 25 bp, Canis familiaris
gggtggccatgccgggcttcgcggc

SEQ ID NO: 997 - DNA, 24 bp, Canis familiaris
gggtggccatgccgggcttcgcgg

SEQ ID NO: 998 - DNA, 23 bp, Canis familiaris
gggtggccatgccgggcttcgcg

SEQ ID NO: 999 - DNA, 22 bp, Canis familiaris
gggtggccatgccgggcttcgc

SEQ ID NO: 1000 - DNA, 21 bp, Canis familiaris
gggtggccatgccgggcttcg

SEQ ID NO: 1001 - DNA, 20 bp, Canis familiaris
gggtggccatgccgggcttc

SEQ ID NO: 1002 - DNA, 22 bp, Canis familiaris
ggccatgccgggcttcgcggcg

SEQ ID NO: 1003 - DNA, 21 bp, Canis familiaris
ggccatgccgggcttcgcggc

SEQ ID NO: 1004 - DNA, 20 bp, Canis familiaris
ggccatgccgggcttcgcgg

SEQ ID NO: 1005 - DNA, 25 bp, Canis familiaris
ccacctcctccatggctgctctgcg

SEQ ID NO: 1006 - DNA, 24 bp, Canis familiaris
ccacctcctccatggctgctctgc

SEQ ID NO: 1007 - DNA, 23 bp, Canis familiaris
ccacctcctccatggctgctctg

SEQ ID NO: 1008 - DNA, 22 bp, Canis familiaris
ccacctcctccatggctgctct

SEQ ID NO: 1009 - DNA, 21 bp, Canis familiaris
ccacctcctccatggctgctc

SEQ ID NO: 1010 - DNA, 20 bp, Canis familiaris
ccacctcctccatggctgct

SEQ ID NO: 1011 - DNA, 25 bp, Canis familiaris
cctcctccatggctgctctgcgcag

SEQ ID NO: 1012 - DNA, 24 bp, Canis familiaris
cctcctccatggctgctctgcgca

SEQ ID NO: 1013 - DNA, 23 bp, Canis familiaris
cctcctccatggctgctctgcgc

SEQ ID NO: 1014 - DNA, 22 bp, Canis familiaris
cctcctccatggctgctctgcg

SEQ ID NO: 1015 - DNA, 21 bp, Canis familiaris
cctcctccatggctgctctgc

SEQ ID NO: 1016 - DNA, 20 bp, Canis familiaris
cctcctccatggctgctctg

SEQ ID NO: 1017 - DNA, 23 bp, Canis familiaris
cctccatggctgctctgcgcagg

SEQ ID NO: 1018 - DNA, 20 bp, Canis familiaris
ccatggctgctctgcgcagg

SEQ ID NO: 1019 - DNA, 25 bp, Canis familiaris
cgccgtctaccagcgccatggtcct

SEQ ID NO: 1020 - DNA, 24 bp, Canis familiaris
cgccgtctaccagcgccatggtcc

SEQ ID NO: 1021 - DNA, 23 bp, Canis familiaris
cgccgtctaccagcgccatggtc

SEQ ID NO: 1022 - DNA, 22 bp, Canis familiaris
cgccgtctaccagcgccatggt

SEQ ID NO: 1023 - DNA, 21 bp, Canis familiaris
cgccgtctaccagcgccatgg

SEQ ID NO: 1024 - DNA, 20 bp, Canis familiaris
cgccgtctaccagcgccatg

SEQ ID NO: 1025 - DNA, 25 bp, Canis familiaris
gccgtctaccagcgccatggtcctg

SEQ ID NO: 1026 - DNA, 24 bp, Canis familiaris
gccgtctaccagcgccatggtcct

SEQ ID NO: 1027 - DNA, 23 bp, Canis familiaris
gccgtctaccagcgccatggtcc

SEQ ID NO: 1028 - DNA, 22 bp, Canis familiaris
gccgtctaccagcgccatggtc

SEQ ID NO: 1029 - DNA, 21 bp, Canis familiaris
gccgtctaccagcgccatggt

SEQ ID NO: 1030 - DNA, 20 bp, Canis familiaris
gccgtctaccagcgccatgg

SEQ ID NO: 1031 - DNA, 19 bp, Canis familiaris
gccgtctaccagcgccatg

SEQ ID NO: 1032 - DNA, 25 bp, Canis familiaris
ccgtctaccagcgccatggtcctgc

SEQ ID NO: 1033 - DNA, 24 bp, Canis familiaris
ccgtctaccagcgccatggtcctg

SEQ ID NO: 1034 - DNA, 23 bp, Canis familiaris
ccgtctaccagcgccatggtcct

SEQ ID NO: 1035 - DNA, 22 bp, Canis familiaris
ccgtctaccagcgccatggtcc

SEQ ID NO: 1036 - DNA, 21 bp, Canis familiaris
ccgtctaccagcgccatggtc

SEQ ID NO: 1037 - DNA, 20 bp, Canis familiaris
ccgtctaccagcgccatggt

SEQ ID NO: 1038 - DNA, 22 bp, Canis familiaris
ccgatgacatccttctccgatg

SEQ ID NO: 1039 - DNA, 21 bp, Canis familiaris
ccgatgacatccttctccgat

SEQ ID NO: 1040 - DNA, 20 bp, Canis familiaris
ccgatgacatccttctccga

SEQ ID NO: 1041 - DNA, 19 bp, Canis familiaris
ccgatgacatccttctccg

SEQ ID NO: 1042 - DNA, 25 bp, Canis familiaris
cggcatcttgggtgcctaccctagg

SEQ ID NO: 1043 - DNA, 24 bp, Canis familiaris
cggcatcttgggtgcctaccctag

SEQ ID NO: 1044 - DNA, 23 bp, Canis familiaris
cggcatcttgggtgcctacccta

SEQ ID NO: 1045 - DNA, 22 bp, Canis familiaris
cggcatcttgggtgcctaccct

SEQ ID NO: 1046 - DNA, 21 bp, Canis familiaris
cggcatcttgggtgcctaccc

SEQ ID NO: 1047 - DNA, 20 bp, Canis familiaris
cggcatcttgggtgcctacc

SEQ ID NO: 1048 - DNA, 25 bp, Canis familiaris
gggtctgcgccgctcctatacatcg

SEQ ID NO: 1049 - DNA, 24 bp, Canis familiaris
gggtctgcgccgctcctatacatc

SEQ ID NO: 1050 - DNA, 23 bp, Canis familiaris
gggtctgcgccgctcctatacat

SEQ ID NO: 1051 - DNA, 22 bp, Canis familiaris
gggtctgcgccgctcctataca

SEQ ID NO: 1052 - DNA, 21 bp, Canis familiaris
gggtctgcgccgctcctatac

SEQ ID NO: 1053 - DNA, 20 bp, Canis familiaris
gggtctgcgccgctcctata

SEQ ID NO: 1054 - DNA, 25 bp, Canis familiaris
gcgccgctcctctacatcgaacacc

SEQ ID NO: 1055 - DNA, 24 bp, Canis familiaris
gcgccgctcctctacatcgaacac

SEQ ID NO: 1056 - DNA, 23 bp, Canis familiaris
gcgccgctcctctacatcgaaca

SEQ ID NO: 1057 - DNA, 22 bp, Canis familiaris
gcgccgctcctctacatcgaac

SEQ ID NO: 1058 - DNA, 21 bp, Canis familiaris
gcgccgctcctctacatcgaa

SEQ ID NO: 1059 - DNA, 20 bp, Canis familiaris
gcgccgctcctctacatcga

SEQ ID NO: 1060 - DNA, 24 bp, Canis familiaris
cgccgctcctctacatcgaacacc

SEQ ID NO: 1061 - DNA, 23 bp, Canis familiaris
cgccgctcctctacatcgaacac

SEQ ID NO: 1062 - DNA, 22 bp, Canis familiaris
cgccgctcctctacatcgaaca

SEQ ID NO: 1063 - DNA, 21 bp, Canis familiaris
cgccgctcctctacatcgaac

SEQ ID NO: 1064 - DNA, 20 bp, Canis familiaris
cgccgctcctctacatcgaa

SEQ ID NO: 1065 - DNA, 23 bp, Canis familiaris
gccgctcctctacatcgaacacc

SEQ ID NO: 1066 - DNA, 22 bp, Canis familiaris
gccgctcctctacatcgaacac

SEQ ID NO: 1067 - DNA, 21 bp, Canis familiaris
gccgctcctctacatcgaaca

SEQ ID NO: 1068 - DNA, 20 bp, Canis familiaris
gccgctcctctacatcgaac

SEQ ID NO: 1069 - DNA, 22 bp, Canis familiaris
ccgctcctctacatcgaacacc

SEQ ID NO: 1070 - DNA, 21 bp, Canis familiaris
ccgctcctctacatcgaacac

SEQ ID NO: 1071 - DNA, 20 bp, Canis familiaris
ccgctcctctacatcgaaca

SEQ ID NO: 1072 - DNA, 20 bp, Canis familiaris
cgctcctctacatcgaacac

SEQ ID NO: 1073 - DNA, 25 bp, Canis familiaris
gcgcccgtgcatggtgccggctcag

SEQ ID NO: 1074 - DNA, 24 bp, Canis familiaris
gcgcccgtgcatggtgccggctca

SEQ ID NO: 1075 - DNA, 23 bp, Canis familiaris
gcgcccgtgcatggtgccggctc

SEQ ID NO: 1076 - DNA, 22 bp, Canis familiaris
gcgcccgtgcatggtgccggct

SEQ ID NO: 1077 - DNA, 21 bp, Canis familiaris
gcgcccgtgcatggtgccggc

SEQ ID NO: 1078 - DNA, 20 bp, Canis familiaris
gcgcccgtgcatggtgccgg

SEQ ID NO: 1079 - DNA, 25 bp, Canis familiaris
cgcccgtgcatggtgccggctcagg

SEQ ID NO: 1080 - DNA, 24 bp, Canis familiaris
cgcccgtgcatggtgccggctcag

SEQ ID NO: 1081 - DNA, 23 bp, Canis familiaris
cgcccgtgcatggtgccggctca

SEQ ID NO: 1082 - DNA, 22 bp, Canis familiaris
cgcccgtgcatggtgccggctc

SEQ ID NO: 1083 - DNA, 21 bp, Canis familiaris
cgcccgtgcatggtgccggct

SEQ ID NO: 1084 - DNA, 20 bp, Canis familiaris
cgcccgtgcatggtgccggc

SEQ ID NO: 1085 - DNA, 25 bp, Canis familiaris
gcccgtgcatggtgccggctcaggg

SEQ ID NO: 1086 - DNA, 24 bp, Canis familiaris
gcccgtgcatggtgccggctcagg

SEQ ID NO: 1087 - DNA, 23 bp, Canis familiaris
gcccgtgcatggtgccggctcag

SEQ ID NO: 1088 - DNA, 22 bp, Canis familiaris
gcccgtgcatggtgccggctca

SEQ ID NO: 1089 - DNA, 21 bp, Canis familiaris
gcccgtgcatggtgccggctc

SEQ ID NO: 1090 - DNA, 20 bp, Canis familiaris
gcccgtgcatggtgccggct

SEQ ID NO: 1091 - DNA, 24 bp, Canis familiaris
cccgtgcatggtgccggctcaggg

SEQ ID NO: 1092 - DNA, 23 bp, Canis familiaris
cccgtgcatggtgccggctcagg

SEQ ID NO: 1093 - DNA, 22 bp, Canis familiaris
cccgtgcatggtgccggctcag

SEQ ID NO: 1094 - DNA, 21 bp, Canis familiaris
cccgtgcatggtgccggctca

SEQ ID NO: 1095 - DNA, 20 bp, Canis familiaris
cccgtgcatggtgccggctc

SEQ ID NO: 1096 - DNA, 23 bp, Canis familiaris
ccgtgcatggtgccggctcaggg

SEQ ID NO: 1097 - DNA, 22 bp, Canis familiaris
ccgtgcatggtgccggctcagg

SEQ ID NO: 1098 - DNA, 21 bp, Canis familiaris
ccgtgcatggtgccggctcag

SEQ ID NO: 1099 - DNA, 20 bp, Canis familiaris
ccgtgcatggtgccggctca

SEQ ID NO: 1100 - DNA, 25 bp, Canis familiaris
gcagattcagaatacatgccagctc

SEQ ID NO: 1101 - DNA, 24 bp, Canis familiaris
gcagattcagaatacatgccagct

SEQ ID NO: 1102 - DNA, 23 bp, Canis familiaris
gcagattcagaatacatgccagc

SEQ ID NO: 1103 - DNA, 22 bp, Canis familiaris
gcagattcagaatacatgccag

SEQ ID NO: 1104 - DNA, 21 bp, Canis familiaris
gcagattcagaatacatgcca

SEQ ID NO: 1105 - DNA, 20 bp, Canis familiaris
gcagattcagaatacatgcc

SEQ ID NO: 1106 - DNA, 25 bp, Canis familiaris
atacatgccagctcatcatcgaccc

SEQ ID NO: 1107 - DNA, 22 bp, Canis familiaris
catgccagctcatcatcgaccc

SEQ ID NO: 1108 - DNA, 21 bp, Canis familiaris
atgccagctcatcatcgaccc

SEQ ID NO: 1109 - DNA, 25 bp, Canis familiaris
gcaagtggcataagcaggctacagg

SEQ ID NO: 1110 - DNA, 24 bp, Canis familiaris
gcaagtggcataagcaggctacag

SEQ ID NO: 1111 - DNA, 23 bp, Canis familiaris
gcaagtggcataagcaggctaca

SEQ ID NO: 1112 - DNA, 22 bp, Canis familiaris
gcaagtggcataagcaggctac

SEQ ID NO: 1113 - DNA, 21 bp, Canis familiaris
gcaagtggcataagcaggcta

SEQ ID NO: 1114 - DNA, 20 bp, Canis familiaris
gcaagtggcataagcaggct

SEQ ID NO: 1115 - DNA, 25 bp, Canis familiaris
gggcgggcatggcgcgggcggcggc

SEQ ID NO: 1116 - DNA, 24 bp, Canis familiaris
gggcgggcatggcgcgggcggcgg

SEQ ID NO: 1117 - DNA, 23 bp, Canis familiaris
gggcgggcatggcgcgggcggcg

SEQ ID NO: 1118 - DNA, 22 bp, Canis familiaris
gggcgggcatggcgcgggcggc

SEQ ID NO: 1119 - DNA, 21 bp, Canis familiaris
gggcgggcatggcgcgggcgg

SEQ ID NO: 1120 - DNA, 20 bp, Canis familiaris
gggcgggcatggcgcgggcg

SEQ ID NO: 1121 - DNA, 19 bp, Canis familiaris
gggcgggcatggcgcgggc

SEQ ID NO: 1122 - DNA, 18 bp, Canis familiaris
gggcgggcatggcgcggg

SEQ ID NO: 1123 - DNA, 17 bp, Canis familiaris
gggcgggcatggcgcgg

SEQ ID NO: 1124 - DNA, 16 bp, Canis familiaris
gggcgggcatggcgcg

SEQ ID NO: 1125 - DNA, 15 bp, Canis familiaris
gggcgggcatggcgc

SEQ ID NO: 1126 - DNA, 14 bp, Canis familiaris
gggcgggcatggcg

SEQ ID NO: 1127 - DNA, 13 bp, Canis familiaris
ggcgggcatggcg

SEQ ID NO: 1128 - DNA, 23 bp, Canis familiaris
cctccagctccatgtcgctgccg

SEQ ID NO: 1129 - DNA, 21 bp, Canis familiaris
cctccagctccatgtcgctgc

SEQ ID NO: 1130 - DNA, 22 bp, Canis familiaris
cctccagctccatgtcgctgcc

SEQ ID NO: 1131 - DNA, 20 bp, Canis familiaris
cctccagctccatgtcgctg

SEQ ID NO: 1132 - DNA, 19 bp, Canis familiaris
cctccagctccatgtcgct

SEQ ID NO: 1133 - DNA, 23 bp, Canis familiaris
gcggcccctccatgcggaggccg

SEQ ID NO: 1134 - DNA, 22 bp, Canis familiaris
gcggcccctccatgcggaggcc

SEQ ID NO: 1135 - DNA, 21 bp, Canis familiaris
gcggcccctccatgcggaggc

SEQ ID NO: 1136 - DNA, 20 bp, Canis familiaris
gcggcccctccatgcggagg

SEQ ID NO: 1137 - DNA, 19 bp, Canis familiaris
gcggcccctccatgcggag

SEQ ID NO: 1138 - DNA, 23 bp, Canis familiaris
cggcccctccatgcggaggccgc

SEQ ID NO: 1139 - DNA, 22 bp, Canis familiaris
cggcccctccatgcggaggccg

SEQ ID NO: 1140 - DNA, 21 bp, Canis familiaris
cggcccctccatgcggaggcc

SEQ ID NO: 1141 - DNA, 20 bp, Canis familiaris
cggcccctccatgcggaggc

SEQ ID NO: 1142 - DNA, 20 bp, Canis familiaris
cggcccctccatgcggaggc

SEQ ID NO: 1143 - DNA, 19 bp, Canis familiaris
cggcccctccatgcggagg

SEQ ID NO: 1144 - DNA, 22 bp, Canis familiaris
ggcccctccatgcggaggccgc

SEQ ID NO: 1145 - DNA, 21 bp, Canis familiaris
ggcccctccatgcggaggccg

SEQ ID NO: 1146 - DNA, 24 bp, Canis familiaris
gcggcccctccatgcggaggccgc

SEQ ID NO: 1147 - DNA, 20 bp, Canis familiaris
ggcccctccatgcggaggcc

SEQ ID NO: 1148 - DNA, 19 bp, Canis familiaris
ggcccctccatgcggaggc

SEQ ID NO: 1149 - DNA, 21 bp, Canis familiaris
gcccctccatgcggaggccgc

SEQ ID NO: 1150 - DNA, 20 bp, Canis familiaris
gcccctccatgcggaggccg

SEQ ID NO: 1151 - DNA, 19 bp, Canis familiaris
gcccctccatgcggaggcc

SEQ ID NO: 1152 - DNA, 24 bp, Canis familiaris
gcggcccctccatgcggaggccgc

SEQ ID NO: 1153 - DNA, 25 bp, Canis familiaris
cgagtgaacgcatgcgcaaagcgcg

SEQ ID NO: 1154 - DNA, 24 bp, Canis familiaris
cgagtgaacgcatgcgcaaagcgc

SEQ ID NO: 1155 - DNA, 23 bp, Canis familiaris
cgagtgaacgcatgcgcaaagcg

SEQ ID NO: 1156 - DNA, 22 bp, Canis familiaris
cgagtgaacgcatgcgcaaagc

SEQ ID NO: 1157 - DNA, 21 bp, Canis familiaris
cgagtgaacgcatgcgcaaag

SEQ ID NO: 1158 - DNA, 20 bp, Canis familiaris
cgagtgaacgcatgcgcaaa

SEQ ID NO: 1159 - DNA, 21 bp, Canis familiaris
acgcatgcgcaaagcgcgccg

SEQ ID NO: 1160 - DNA, 21 bp, Canis familiaris
cgcatgcgcaaagcgcgccgt

SEQ ID NO: 1161 - DNA, 20 bp, Canis familiaris
cgcatgcgcaaagcgcgccg

SEQ ID NO: 1162 - DNA, 22 bp, Canis familiaris
gcaactacagccatgatacagg

SEQ ID NO: 1163 - DNA, 22 bp, Canis familiaris
cccgccatgggccggcgcgggc

SEQ ID NO: 1164 - DNA, 22 bp, Canis familiaris
cacagtgaacatctttcctccc

SEQ ID NO: 1165 - DNA, 23 bp, Canis familiaris
ccccatgctgacgggtcagagcc

SEQ ID NO: 1166 - DNA, 21 bp, Canis familiaris
ccccatgctgacgggtcagag

SEQ ID NO: 1167 - DNA, 25 bp, Canis familiaris
cccaggtagtggccattctcttccc

SEQ ID NO: 1168 - DNA, 22 bp, Canis familiaris
ctcatttctctgtctgctgcgg

SEQ ID NO: 1169 - DNA, 22 bp, Canis familiaris
ggtatttctctgtctgctgagg

SEQ ID NO: 1170 - DNA, 23 bp, Canis familiaris
ctagtaacttcataatgaatgtg

SEQ ID NO: 1171 - DNA, 21 bp, Canis familiaris
cgggcttctccttgccatggc

SEQ ID NO: 1172 - DNA, 21 bp, Canis familiaris
tctccttgccatggcagaggg

SEQ ID NO: 1173 - DNA, 21 bp, Canis familiaris
ggagttttgaagtcattttca

SEQ ID NO: 1174 - DNA, 25 bp, Canis familiaris
gcggtccccagcctccgccatcccc

SEQ ID NO: 1175 - DNA, 24 bp, Canis familiaris
gcggtccccagcctccgccatccc

SEQ ID NO: 1176 - DNA, 23 bp, Canis familiaris
gcggtccccagcctccgccatcc

SEQ ID NO: 1177 - DNA, 22 bp, Canis familiaris
gcggtccccagcctccgccatc

SEQ ID NO: 1178 - DNA, 21 bp, Canis familiaris
gcggtccccagcctccgccat

SEQ ID NO: 1179 - DNA, 20 bp, Canis familiaris
gcggtccccagcctccgcca

SEQ ID NO: 1180 - DNA, 25 bp, Canis familiaris
cggtccccagcctccgccatcccca

SEQ ID NO: 1181 - DNA, 24 bp, Canis familiaris
cggtccccagcctccgccatcccc

SEQ ID NO: 1182 - DNA, 23 bp, Canis familiaris
cggtccccagcctccgccatccc

SEQ ID NO: 1183 - DNA, 22 bp, Canis familiaris
cggtccccagcctccgccatcc

SEQ ID NO: 1184 - DNA, 21 bp, Canis familiaris
cggtccccagcctccgccatc

SEQ ID NO: 1185 - DNA, 20 bp, Canis familiaris
cggtccccagcctccgccat

SEQ ID NO: 1186 - DNA, 22 bp, Canis familiaris
ccccagcctccgccatccccaa

SEQ ID NO: 1187 - DNA, 21 bp, Canis familiaris
ccccagcctccgccatcccca

SEQ ID NO: 1188 - DNA, 21 bp, Canis familiaris
ccccagcctccgccatcccca

SEQ ID NO: 1189 - DNA, 21 bp, Canis familiaris
cccagcctccgccatccccaa

SEQ ID NO: 1190 - DNA, 22 bp, Canis familiaris
cccagcctccgccatccccaac

SEQ ID NO: 1191 - DNA, 23 bp, Canis familiaris
ccgccatccccaacacgctaccc

SEQ ID NO: 1192 - DNA, 22 bp, Canis familiaris
ccgccatccccaacacgctacc

SEQ ID NO: 1193 - DNA, 21 bp, Canis familiaris
ccgccatccccaacacgctac

SEQ ID NO: 1194 - DNA, 20 bp, Canis familiaris
ccgccatccccaacacgcta

SEQ ID NO: 1195 - DNA, 24 bp, Canis familiaris
cggcgagcgccatggctccagtga

SEQ ID NO: 1196 - DNA, 23 bp, Canis familiaris
cggcgagcgccatggctccagtg

SEQ ID NO: 1197 - DNA, 22 bp, Canis familiaris
cggcgagcgccatggctccagt

SEQ ID NO: 1198 - DNA, 21 bp, Canis familiaris
cggcgagcgccatggctccag

SEQ ID NO: 1199 - DNA, 24 bp, Canis familiaris
ggcgagcgccatggctccagtgac

SEQ ID NO: 1200 - DNA, 23 bp, Canis familiaris
ggcgagcgccatggctccagtga

SEQ ID NO: 1201 - DNA, 22 bp, Canis familiaris
ggcgagcgccatggctccagtg

SEQ ID NO: 1202 - DNA, 21 bp, Canis familiaris
ggcgagcgccatggctccagt

SEQ ID NO: 1203 - DNA, 20 bp, Canis familiaris
ggcgagcgccatggctccag

SEQ ID NO: 1204 - DNA, 24 bp, Canis familiaris
gcgagcgccatggctccagtgacg

SEQ ID NO: 1205 - DNA, 23 bp, Canis familiaris
gcgagcgccatggctccagtgac

SEQ ID NO: 1206 - DNA, 22 bp, Canis familiaris
gcgagcgccatggctccagtga

SEQ ID NO: 1207 - DNA, 21 bp, Canis familiaris
gcgagcgccatggctccagtg

SEQ ID NO: 1208 - DNA, 22 bp, Canis familiaris
gcgccatggctccagtgacgcg

SEQ ID NO: 1209 - DNA, 21 bp, Canis familiaris
gcgccatggctccagtgacgc

SEQ ID NO: 1210 - DNA, 21 bp, Canis familiaris
cgccatggctccagtgacgcg

SEQ ID NO: 1211 - DNA, 20 bp, Canis familiaris
cgccatggctccagtgacgc

SEQ ID NO: 1212 - DNA, 24 bp, Canis familiaris
gggtccgccatcgccaagccaccg

SEQ ID NO: 1213 - DNA, 23 bp, Canis familiaris
gggtccgccatcgccaagccacc

SEQ ID NO: 1214 - DNA, 22 bp, Canis familiaris
gggtccgccatcgccaagccac

SEQ ID NO: 1215 - DNA, 21 bp, Canis familiaris
gggtccgccatcgccaagcca

SEQ ID NO: 1216 - DNA, 24 bp, Canis familiaris
tccgccatcgccaagccaccgcgc

SEQ ID NO: 1217 - DNA, 23 bp, Canis familiaris
tccgccatcgccaagccaccgcg

SEQ ID NO: 1218 - DNA, 22 bp, Canis familiaris
tccgccatcgccaagccaccgc

SEQ ID NO: 1219 - DNA, 21 bp, Canis familiaris
tccgccatcgccaagccaccg

SEQ ID NO: 1220 - DNA, 23 bp, Canis familiaris
ccgccatcgccaagccaccgcgc

SEQ ID NO: 1221 - DNA, 22 bp, Canis familiaris
ccgccatcgccaagccaccgcg

SEQ ID NO: 1222 - DNA, 21 bp, Canis familiaris
ccgccatcgccaagccaccgc

SEQ ID NO: 1223 - DNA, 20 bp, Canis familiaris
ccgccatcgccaagccaccg